(19) United States
(12) Patent Application Publication
(10) Pub. No.: US 2021/0062260 A1
(43) Pub. Date: Mar. 4, 2021

(54) GENETIC MARKERS AND USES THEREFOR

(71) Applicant: LIVESTOCK IMPROVEMENT CORPORATION LIMITED, Hamilton (NZ)

(72) Inventors: Mathew Douglas LITTLEJOHN, Hamilton (NZ); Michael Dominic KEEHAN, Hamilton (NZ)

(73) Assignee: LIVESTOCK IMPROVEMENT CORPORATION LIMITED, Hamilton (NZ)

(21) Appl. No.: 16/956,806

(22) PCT Filed: Dec. 21, 2018
(86) PCT No.: PCT/NZ2018/050185

§ 371 Date: Jun. 22, 2020

(30) Foreign Application Priority Data
Dec. 21, 2017 (NZ) 738338

(51) Int. Cl. C12Q 1/6876; A01K 67/02

(52) U.S. Cl. CPC C12Q 1/6876; A01K 67/02; C12Q 2600/158; C12Q 2600/124; C12Q 2600/156; A01K 2227/101

(57) ABSTRACT

The present invention generally relates to methods of identifying whether or not an animal carries a biological marker linked to animal productivity, more particularly, but not exclusively, to methods of identifying whether or not an animal carries a biological marker having a deleterious effect on animal productivity. The invention also relates to methods for selecting or rejecting one or more animals, cells or embryos, animal evaluation, breeding animals, and herd formation. The invention also relates to biological markers suitable for use in such methods. In particular, the present invention relates to genetic variations which disrupt the PLCD4 gene.

### FIELD

The present invention generally relates to methods of identifying whether or not an animal carries a biological marker linked to animal productivity, more particularly, but not exclusively, to methods of identifying whether or not an animal carries a biological marker having a deleterious effect on animal productivity. The invention also relates to methods for selecting or rejecting one or more animals, cells or embryos, animal evaluation, breeding animals, and herd formation. The invention also relates to biological markers suitable for use in such methods.

### BACKGROUND

Dairy animals are typically assessed to determine their suitability, or otherwise, for a particular purpose including breeding, inclusion in a herd, and milk production, on the basis of a number of different traits relating to productivity. In New Zealand, for example, up to 25 different traits may be assessed. These traits include production traits including milk protein yield, milk fat yield, milk volume, liveweight, and survival, and traits other than production (TOP) including milking speed, temperament and conformation, for example. In various combinations assessment of these traits are used to determine or estimate an animal's value or worth to a farmer. In New Zealand, for example, assessment of and scoring an animal on the basis of a combination of such traits is used to determine or estimate Breeding Worth and/or Production Worth, for example. Similar evaluation systems may be used in other markets also.

Traditional methods of assessment, herd formation and breeding programmes for livestock focused on assessment and selection of animals based on the presence of certain phenotypic characteristics. More recent methods of assessment and selection have incorporated criteria based on genotypic characteristics associated with a desirable trait, or assessment and rejection of animals based on genotypic characteristics associated with an undesirable trait. Selection or rejection of animals on the basis of genotypic characteristics allows for earlier and more specific selection of animals of interest than does selection on the basis of phenotypic characteristics.

A number of genetic markers are known to be associated (either positively or negatively) with productivity in dairy animals and are used to assess animals, calculate breeding values, Breeding Worth, Production Worth and/or Lactation Worth and make selection decisions. However, there is a need to identify additional markers which can be used to increase the accuracy of assessment and selection methods.

### Object

It is an object of the present invention to provide one or more of a method for determining whether or not a non-human animal, cell or embryo carries a biological marker linked to a deleterious effect on productivity, worth and/or one or more related traits, a method for determining whether or not a non-human cell or embryo is suitable for being used to produce an animal that does not carry a biological marker linked to a deleterious effect on productivity, worth, and/or one or more related traits, a method for estimating the worth of a non-human animal, a method for selecting or rejecting a non-human animal, cell or embryo, a method for breeding non-human animals, a method of cloning a non-human animal, a method of forming a herd, a herd formed by a method of the invention, a method for generating a non-human animal, cell or embryo, non-human animals, cells and embryos produced or selected by a method of the invention, uses for said non-human animals, cells and embryos, a nucleic acid, a peptide and/or at least to provide the public with a useful choice.

### STATEMENT OF INVENTION

The inventors contemplate that an alteration or variation in the PLCD4 gene which disrupts the gene can have a deleterious effect on productivity in an animal which is homozygous for that alteration or variation. Methods involving the analysis of such alterations or variations can be used for the purposes of selecting, screening and breeding animals, farm management, and for estimating an animal's worth to a particular industry, for example.

In a first aspect, the invention provides a method for identifying whether or not a non-human animal, cell or embryo carries a genetic marker linked to a deleterious effect on productivity and/or worth of an animal, the method comprising at least the step of analysing a nucleic acid of said animal, cell or embryo to identify whether or not it includes a genetic variation which disrupts the PLCD4 gene and/or a genetic marker in linkage disequilibrium therewith, wherein where the nucleic acid includes a genetic variation which disrupts the PLCD4 gene and/or a genetic marker in linkage disequilibrium therewith, it is identified to carry a genetic marker linked to a deleterious effect on productivity and/or worth of an animal.

In one embodiment of the first aspect, the method comprises as least the step of analysing the nucleic acid to identify whether or not it includes a genetic variation at a position corresponding to position 107313998 of chromosome 2 of Bos taurus.

In one embodiment of the first aspect, the method comprises at least the step of analysing the nucleic acid to identify whether or not it includes the genetic marker 107313998A on chromosome 2 of Bos taurus and/or a genetic marker in linkage disequilibrium therewith. In one embodiment, where it includes the genetic marker 107313998A and/or a genetic marker in linkage disequilibrium therewith the animal, cell or embryo is identified to carry a genetic marker linked to a deleterious effect on productivity and/or worth of an animal. In one embodiment, where it includes the genetic marker 107313998G and/or a genetic marker in linkage disequilibrium therewith the animal, cell or embryo is identified not to carry a genetic marker linked to a deleterious effect on productivity and/or worth of an animal.

In one embodiment, the method of the first aspect is used for selecting or rejecting a non-human animal, cell or embryo and/or estimating the worth of a non-human animal and/or its offspring.

In one embodiment, the method of the first aspect is used to identify whether or not an animal is suitable for (or at least more or less suitable for) inclusion in a herd, production purposes and/or breeding purposes. In another embodiment, the method of the first aspect is used to identify whether or not a cell or embryo is suitable for (or at least more or less suitable for) producing an animal which is suitable for (or at least more suitable for) inclusion in a herd, production purposes and/or breeding purposes.

In one embodiment, the method of the first aspect comprises identifying whether or not the animal, cell or embryo is heterozygous or homozygous for a genetic marker linked to a deleterious effect on productivity and/or worth of an animal and/or a genetic marker in linkage disequilibrium therewith.

In a second aspect, the invention provides a method for selecting or rejecting a non-human animal, cell or embryo the method comprising selecting the non-human animal, cell or embryo where it has been identified not to carry a genetic marker linked to a deleterious effect on productivity and/or worth of an animal according to a method of the first aspect.

In one embodiment of the second aspect, the invention provides a method for selecting or rejecting a non-human animal, cell or embryo, the method comprising at least the step of analysing a nucleic acid of said animal, cell or embryo to identify whether or not it includes a genetic variation which disrupts the PLCD4 gene and/or a genetic marker in linkage disequilibrium therewith.

In one embodiment of the second aspect, the method comprises at least the steps of: a) performing a method of the first aspect; and, b) selecting or rejecting the non-human animal, cell or embryo using the results of step a).

In one embodiment of the second aspect, an animal, cell or embryo is selected if it does not have a genetic variation which disrupts the PLCD4 gene and/or a genetic marker in linkage disequilibrium therewith. In one embodiment, an animal, cell or embryo is rejected if it has a genetic variation which disrupts the PLCD4 gene and/or a genetic marker in linkage disequilibrium therewith. In another embodiment, if the animal, cell or embryo has been identified to have the wild-type nucleotide sequence of PLCD4 the animal, cell or embryo is selected.

In one embodiment of the second aspect, the method comprises at least the step of analysing a nucleic acid of said animal, cell or embryo to identify whether or not it includes a variation at a position corresponding to position 107313998 on chromosome 2 of Bos taurus and/or a genetic marker in linkage disequilibrium therewith. In one embodiment, the variation is a nucleotide substitution.

In one embodiment of the second aspect, the method comprises at least the step of analysing a nucleic acid of said animal, cell or embryo to identify whether or not it includes the genetic marker 107313998A on chromosome 2 of Bos taurus and/or a genetic marker in linkage disequilibrium therewith.

In one embodiment, the method of the second aspect comprises at least the steps of:
a) analysing a nucleic acid of said animal, cell or embryo to identify whether or not it includes the genetic marker 107313998A on chromosome 2 of Bos taurus and/or a genetic marker in linkage disequilibrium therewith; and,
b) selecting or rejecting an animal, cell or embryo based on the presence or absence of the genetic marker 107313998A on chromosome 2 of Bos taurus and/or a genetic marker in linkage disequilibrium therewith.

In one embodiment of the second aspect, where the nucleic acid includes the genetic marker 107313998A and/or a genetic marker in linkage disequilibrium therewith the animal, cell or embryo is identified to carry a genetic marker linked to a deleterious effect on productivity and/or worth of an animal and the animal.

In one embodiment of the second aspect, where the nucleic acid includes the genetic marker 107313998A and/or a genetic marker in linkage disequilibrium therewith the animal, cell or embryo is rejected. In one embodiment, where the nucleic acid includes the genetic marker 107313998G the animal, cell or embryo is selected.

In one embodiment of the second aspect, the method further comprises identifying whether or not the animal, cell or embryo is heterozygous or homozygous. In one embodiment, an animal, cell or embryo is rejected if it is heterozygous for a genetic marker linked to a deleterious effect on productivity and/or worth of an animal. In an alternative embodiment, an animal, cell or embryo is selected if it is heterozygous for a genetic marker linked to a deleterious effect on productivity and/or worth of an animal. In another embodiment, an animal, cell or embryo is rejected if it is homozygous for a genetic marker linked to a deleterious effect on productivity and/or worth of an animal.

In one embodiment of the second aspect, the method is performed for the purpose of selecting or rejecting a non-human animal for production purposes. In one embodiment, the method is performed for the purpose of selecting or rejecting a non-human animal for milking purposes. In one embodiment, the method is performed for the purpose of selecting or rejecting a non-human animal for beef farming. In another embodiment, the method is performed for the purpose of selecting or rejecting a non-human animal for breeding purposes. In one embodiment, the method is performed for the purpose of selecting or rejecting a non-human animal for inclusion in a herd.

In other embodiments of the second aspect, the method is performed for the purpose of selecting or rejecting non-human cell or embryo for use in cloning a non-human animal and/or breeding a non-human animal. In one embodiment, breeding an animal may involve IVF.

In a third aspect, the invention provides a method for estimating the worth of a non-human animal and/or its offspring, the method comprising at least the steps of: a) performing a method of the first aspect; and, b) estimating the worth of the animal and/or its offspring using the results of step a). For example, the method may comprise analysing a nucleic acid of said animal to identify whether or not it includes a genetic variation which disrupts the PLCD4 gene and/or a genetic marker in linkage disequilibrium therewith.

In one embodiment of the third aspect, when a genetic variation which disrupts the PLCD4 gene and/or a genetic marker in linkage disequilibrium therewith is present the animal is identified to carry a genetic marker linked to a deleterious effect on productivity and/or worth of an animal.

In one embodiment of the third aspect, the invention provides a method for estimating the worth of a non-human animal and/or its offspring, the method comprising at least the step of analysing a nucleic acid of the animal to identify whether or not it includes a variation at a position corresponding to position 107313998 on chromosome 2 of Bos taurus and/or a genetic marker in linkage disequilibrium thereof. In one embodiment, the variation is substitution of a nucleotide.

In one embodiment of the third aspect, the invention provides a method for estimating the worth of a non-human animal and/or its offspring, the method comprising at least the step of analysing a nucleic acid of the animal to identify whether or not it includes the genetic marker 107313998A on chromosome 2 of Bos taurus and/or a genetic marker in linkage disequilibrium thereof.

In one embodiment of the third aspect, where the genetic marker 107313998A and/or a genetic marker in linkage disequilibrium therewith is present the animal is identified to carry a genetic marker linked to a deleterious effect on productivity and/or worth of an animal. In one embodiment, where the genetic marker 107313998G and/or a genetic marker in linkage disequilibrium therewith is present the animal is identified not to carry a genetic marker linked to a deleterious effect on productivity and/or worth of an animal.

In one embodiment of the third aspect, the method further comprises identifying whether or not the animal is heterozygous or homozygous for a genetic marker linked to a deleterious effect on productivity and/or worth in an animal.

In a fourth aspect, the invention provides a method for breeding a non-human animal which comprises selecting at least a first non-human animal that has been identified not to have a genetic variation which disrupts the PLCD4 gene and/or a genetic marker in linkage disequilibrium therewith and mating said first animal with a second non-human animal.

In one embodiment of the fourth aspect, the method further comprises selecting the second animal where it has been identified not to have a genetic variation which disrupts the PLCD4 gene and/or a genetic marker in linkage disequilibrium therewith.

In one embodiment of the fourth aspect, the invention provides a method for breeding a non-human animal which comprises selecting at least a first non-human animal that does not have a genetic variation at a position corresponding to position 107313998 on chromosome 2 of Bos taurus and/or a genetic marker in linkage disequilibrium therewith and mating said first animal with a second non-human animal. In one embodiment, the method also comprises selecting a second non-human animal that does not have a genetic variation at a position corresponding to position 107313998 on chromosome 2 of Bos taurus and/or a genetic marker in linkage disequilibrium therewith.

In one embodiment of the fourth aspect, the invention provides a method for breeding a non-human animal which comprises selecting the first and/or second non-human animal where it does not have the genetic marker 107313998A and/or a genetic marker in linkage disequilibrium therewith.

In one embodiment of the fourth aspect, the first and/or second animal are selected where it has the genetic marker 107313998G and/or a genetic marker in linkage disequilibrium therewith.

In one embodiment of the fourth aspect, the method comprises the steps of: a) performing a method of the first aspect of the invention for the first and/or second non-human animal; and, b) selecting the first and/or second non-human animal where it is identified not to have a genetic variation which disrupts the PLCD4 gene and/or a genetic marker in linkage disequilibrium therewith.

In one embodiment of the fourth aspect, the method further comprises identifying whether or not the animal is heterozygous or homozygous for a genetic marker linked to a deleterious effect on productivity and/or worth in an animal.

In a fifth aspect, the invention provides a method for breeding a non-human animal, the method comprising at least the step of selecting a first non-human gamete that has been identified not to have a genetic variation which disrupts the PLCD4 gene and/or a genetic marker in linkage disequilibrium therewith and fusing said first gamete with a second non-human gamete to form a zygote.

In one embodiment of the fifth aspect, the method further comprises selecting the second gamete where it has been identified not to have a genetic variation which disrupts the PLCD4 gene and/or a genetic marker in linkage disequilibrium therewith.

In one embodiment of the fifth aspect, the method comprises selecting the first and/or second gamete where it does not have a genetic variation at a position corresponding to position 107313998 on chromosome 2 of Bos taurus and/or a genetic marker in linkage disequilibrium therewith.

In one embodiment of the fifth aspect, the method comprises selecting the first and/or second gamete where it does not have the genetic marker 107313998A and/or a genetic marker in linkage disequilibrium therewith.

In one embodiment of the fifth aspect, the first and/or second gamete is selected where it has the genetic marker 107313998G and/or a genetic marker in linkage disequilibrium therewith.

In one embodiment of the fifth aspect, the method further comprises the steps of a) performing a method of the first aspect of the invention for the first non-human gamete; and, b) selecting the first and/or second non-human gamete if it is identified not to have a genetic variation which disrupts the PLCD4 gene and/or a genetic marker in linkage disequilibrium therewith.

In one embodiment of the fifth aspect, the method further comprises identifying whether or not the gamete is heterozygous or homozygous for a genetic marker linked to a deleterious effect on productivity and/or worth in an animal.

In a sixth aspect, the invention provides a method of breeding a non-human animal, the method comprising at least the step of selecting a non-human embryo that has been identified not to have a genetic variation which disrupts the PLCD4 gene and/or a genetic marker in linkage disequilibrium therewith. A selected embryo is used to breed an animal. In one embodiment, the method further comprises transferring an embryo to a gestational carrier.

In one embodiment of the sixth aspect, the method comprises selecting the embryo where it does not have a genetic variation at a position corresponding to position 107313998 on chromosome 2 of Bos taurus and/or a genetic marker in linkage disequilibrium therewith.

In one embodiment of the sixth aspect, the embryo is selected where it does not have the genetic marker 107313998A and/or a genetic marker in linkage disequilibrium therewith.

In one embodiment of the sixth aspect, the embryo is selected where it has the genetic marker 107313998G and/or a genetic marker in linkage disequilibrium therewith.

In one embodiment of the sixth aspect, the method further comprises the steps of: a) performing a method of the first aspect of the invention for the non-human embryo; and, b) selecting the embryo if it is identified not to have a genetic variation which disrupts the PLCD4 gene and/or a genetic marker in linkage disequilibrium therewith.

In one embodiment of the sixth aspect, the method further comprises identifying whether or not the embryo is heterozygous or homozygous for a genetic marker linked to a deleterious effect on productivity and/or worth in an animal.

In a seventh aspect, the invention provides a method of cloning a non-human animal, the method comprising at least the step of selecting a non-human cell that has been identified not to have a genetic variation which disrupts the PLCD4 gene and/or a genetic marker in linkage disequilibrium therewith. One or more selected cells are used to clone an animal.

In one embodiment of the seventh aspect, a cell is selected where it does not have a genetic alteration at a position corresponding to position 107313998 of chromosome 2 of Bos taurus and/or a genetic marker in linkage disequilibrium therewith.

In one embodiment of the seventh aspect, a cell is selected where it has the genetic marker 107313998A and/or a genetic marker in linkage disequilibrium therewith.

In one embodiment of the seventh aspect, a cell is selected where it has been identified to have the genetic marker 107313998G and/or a genetic marker in linkage disequilibrium therewith.

In one embodiment of the seventh aspect, the method further comprises the steps of: a) performing a method of the first aspect of the invention for the non-human cell; and, b) selecting the cell where it is identified not to have a genetic variation which disrupts the PLCD4 gene and/or a genetic marker in linkage disequilibrium therewith.

In one embodiment of the seventh aspect, the method further comprises identifying whether or not the cell is heterozygous or homozygous for a genetic marker linked to a deleterious effect on productivity and/or worth in an animal.

In one embodiment of the first to seventh aspects, the genetic variation which disrupts the PLCD4 gene and/or a genetic marker in linkage disequilibrium therewith, results in a decrease in the level and/or activity of PLCD4.

In one embodiment of the first to seventh aspects, the genetic variation which disrupts the PLCD4 gene and/or a genetic marker in linkage disequilibrium therewith, is a missense variant.

In one embodiment of the first to seventh aspects, the genetic variation which disrupts the PLCD4 gene is located within a region defined by nucleotides corresponding to positions 107313997 to 107314141 of chromosome 2 of Bos Taurus (which defines exon 8).

In one embodiment of the first to seventh aspects, the methods involve analysing a nucleic acid to identify whether or not it includes two or more genetic variations which disrupt the PLCD4 gene and/or a genetic marker in linkage disequilibrium therewith. In one embodiment, the methods involve analysing a nucleic acid to identify whether or not it includes two or more genetic variations which disrupt the PLCD4 gene and/or two or more genetic markers in linkage disequilibrium therewith.

In one embodiment of the first to seventh aspects, the methods of the invention involve analysing a nucleic acid to identify whether or not it includes a genetic variation which disrupts the PLCD4 gene alone or in combination with a genetic marker in linkage disequilibrium therewith. In one embodiment, the methods of the invention involve analysing only a genetic marker in linkage disequilibrium with a genetic variation which disrupts the PLCD4 gene.

In one embodiment of the first to seventh aspects, the methods of the invention involve analysing a nucleic acid to determine whether or not it includes a genetic variation at a position corresponding to position 107313998 on chromosome 2 of Bos taurus alone or in combination with a genetic marker in linkage disequilibrium therewith. In one embodiment, the methods of the invention involve analysing only a genetic marker in linkage disequilibrium with the genetic variation 107313998 on chromosome 2 of Bos taurus.

In one embodiment of the first to seventh aspects, the methods of the invention involve analysing a nucleic acid to determine whether or not it includes the genetic marker 107313998A on chromosome 2 of Bos taurus alone or in combination with a genetic marker in linkage disequilibrium therewith. In one embodiment, the methods of the invention involve analysing only a genetic marker in linkage disequilibrium with the genetic marker 107313998A on chromosome 2 of Bos taurus.

In one embodiment of the first to seventh aspects, the various markers of use in the invention can be analysed in combination with another biological marker.

In an eighth aspect, the invention provides a method for identifying whether or not a non-human animal, cell or embryo carries a biological marker linked to a deleterious effect on productivity and/or worth of an animal, the method comprising at least the step of analysing a PLCD4, a precursor thereof, an isoform thereof and/or a fragment thereof of said animal, cell or embryo to identify whether or not it includes a variation which disrupts PLCD4, wherein where it includes a variation which disrupts PLCD4, the non-human animal, cell or embryo is determined to carry a biological marker linked to a deleterious effect on productivity and/or worth.

In one embodiment of the eighth aspect, the method comprises at least the step of analysing a PLCD4, a precursor thereof, an isoform thereof and/or a fragment thereof to identify whether or not it includes an amino acid variation at a position corresponding to position 326 of PLCD4. In one embodiment, the amino acid variation is an amino acid substitution. In one embodiment, the method comprises at least the step of analysing a PLCD4, a precursor thereof, an isoform thereof and/or a fragment thereof to identify whether or not it includes the amino acid 326Thr. In one embodiment, where it includes the amino acid 326Thr the animal, cell or embryo is identified to carry a biological marker linked to a deleterious effect on productivity and/or worth of an animal.

In one embodiment, the method of the eighth aspect is used for selecting or rejecting a non-human animal, cell or embryo and/or estimating the worth of a non-human animal and/or its offspring.

In one embodiment, the method of the eighth aspect is used to identify whether or not an animal is suitable for (or at least more or less suitable for) inclusion in a herd, production purposes and/or breeding purposes. In another embodiment, the method of the eighth aspect is used to identify whether or not a cell or embryo is suitable for (or at least more or less suitable for) producing an animal which is suitable for (or at least more suitable for) inclusion in a herd, production purposes and/or breeding purposes.

In a ninth aspect, the invention provides a method for selecting or rejecting a non-human animal, cell or embryo the method comprising selecting the non-human animal, cell or embryo where it has been determined not to carry a biological marker linked to a deleterious effect on productivity and/or worth of an animal according to a method of the eighth aspect.

In one embodiment of the ninth aspect, the invention provides a method for selecting or rejecting a non-human animal, cell or embryo, the method comprising at least the step of analysing a PLCD4, a precursor thereof, an isoform thereof and/or a fragment thereof of said animal, cell or embryo to identify whether or not it includes a variation in the amino acid sequence which disrupts PLCD4.

In one embodiment of the ninth aspect, the method comprises at least the steps of: a) performing a method of the eighth aspect; and, b) selecting or rejecting the non-human animal, cell or embryo using the results of step a).

In one embodiment of the ninth aspect, an animal, cell or embryo is selected if it does not have a variation which disrupts PLCD4. In one embodiment, an animal, cell or embryo is rejected if it has a variation which disrupts PLCD4. In another embodiment, if the animal, cell or embryo has been identified to have the wild-type amino acid sequence of PLCD4 the animal is selected.

In one embodiment of the ninth aspect, where a PLCD4, a precursor thereof, an isoform thereof and/or a fragment thereof comprises an amino acid variation at a position corresponding to position 326 of PLCD4 the animal, cell or embryo is rejected. In one embodiment, the amino acid variation is an amino acid substitution.

In one embodiment of the ninth aspect, the method comprises at least the step of analysing a PLCD4, a precursor thereof, an isoform thereof and/or a fragment thereof of said animal, cell or embryo to identify whether or not it includes the biological marker 326Thr.

In one embodiment of the ninth aspect, the method comprises at least the steps of:
a) analysing a PLCD4, a precursor thereof, an isoform thereof and/or a fragment thereof of said animal, cell or embryo to identify whether or not it includes the biological marker 326Thr; and,
b) selecting or rejecting an animal, cell or embryo based on the presence or absence of the biological marker 326Thr.

In one embodiment of the ninth aspect, where a PLCD4, a precursor thereof, an isoform thereof and/or a fragment thereof comprises 326Thr, the animal, cell or embryo is identified to carry a biological marker linked to a deleterious effect on productivity and/or worth of an animal.

In one embodiment of the ninth aspect, where a PLCD4, a precursor thereof, an isoform thereof and/or a fragment thereof comprises 326Thr, the animal, cell or embryo is rejected. In one embodiment, where a PLCD4, a precursor thereof, an isoform thereof and/or a fragment thereof comprises 326Aa, the animal, cell or embryo is selected. In one embodiment, where a PLCD4, a precursor thereof an isoform thereof and/or a fragment thereof comprises the wild type amino acid sequence of PLCD4 (SEQ ID NO: 3), the animal, cell or embryo is selected.

In one embodiment of the ninth aspect, the method is performed for the purpose of selecting or rejecting a non-human animal for milking purposes. In one embodiment, the method is performed for the purpose of selecting or rejecting a non-human animal for beef farming. In another embodiment, the method is performed for the purpose of selecting or rejecting a non-human animal for breeding purposes. In one embodiment, the method is performed for the purpose of selecting or rejecting a non-human animal for inclusion in a herd.

In other embodiments of the ninth aspect, the method is performed for the purpose of selecting or rejecting a non-human cell or embryo for use in cloning a non-human animal and/or breeding a non-human animal. In one embodiment, breeding an animal may involve IVF.

In a tenth aspect, the invention provides a method for estimating the worth of an animal and/or its offspring, the method comprising at least the steps of: a) performing a method of the eighth aspect; and, b) estimating the worth of the animal and/or its offspring using the results of step a). For example, the method may comprise analysing a PLCD4, a precursor thereof, an isoform thereof and/or a fragment thereof from said animal to identify whether or not it includes a variation which disrupts PLCD4.

In one embodiment of the tenth aspect, where it is identified that the amino acid sequence of PLCD4 includes an amino acid variation which disrupts PLCD4, the animal is identified to carry a biological marker linked to a deleterious effect on productivity and/or worth of an animal. In one embodiment, the amino acid variation is at a position corresponding to position 326 of PLCD4. In one embodiment, the amino acid variation is an amino acid substitution.

In one embodiment of the tenth aspect, the invention provides a method for estimating the worth of a non-human animal and/or its offspring, the method comprising at least the step of analysing a PLCD4, a precursor thereof, an isoform thereof and/or a fragment thereof from said animal to identify whether or not it includes the biological marker 326Thr.

In one embodiment of the tenth aspect, where the biological marker 326Thr is present the animal is identified to carry a biological marker linked to a deleterious effect on productivity and/or worth of an animal. In one embodiment, where the biological marker 326Ala is present the animal is identified not to carry a biological marker linked to a deleterious effect on productivity and/or worth of an animal.

In an eleventh aspect, the invention provides a method for breeding a non-human animal which comprises selecting at least a first animal that has been identified not to have an amino acid variation which disrupts PLCD4 and mating said first animal with a second animal.

In one embodiment of the eleventh aspect, the method further comprises the step of selecting the second animal where it has been identified not to have an amino acid variation which disrupts PLCD4.

In one embodiment of the eleventh aspect, the first and/or second animal is selected where it does not have an amino acid variation at a position corresponding to position 326 of PLCD4.

In one embodiment of the eleventh aspect, the first and/or the second non-human is selected where it does not have the biological marker 326Thr.

In one embodiment of the eleventh aspect, the first and/or second animal are selected where it has the biological marker 326Aa. In one embodiment, the first and/or second animal is selected where it has the wild type amino acid sequence of PLCD4 (SEQ ID No: 3).

In one embodiment of the eleventh aspect, the method further comprises the steps of: a) performing a method of the eighth aspect of the invention for the first and/or second animal; and, b) selecting the first and/or second animal where it is identified not to have an amino acid variation which disrupts PLCD4.

In a twelfth aspect, the invention provides a method for breeding a non-human animal the method comprising at least the step of selecting a first non-human gamete that has been identified not to have an amino acid variation which disrupts PLCD4 and fusing said first gamete with a second non-human gamete to form a zygote.

In one embodiment of the twelfth aspect, the method further comprises selecting the second gamete where it has been identified not to have an amino acid variation which disrupts PLCD4.

In one embodiment of the twelfth aspect, the method comprises selecting the first and/or second gamete where it does not have an amino acid variation at a position corresponding to position 326 of PLCD4.

In one embodiment of the twelfth aspect, the method comprises selecting the first and/or second gamete where it does not have the biological marker 326 hr.

In one embodiment of the twelfth aspect, the first and/or second gamete is selected where is has the biological marker 326Aa. In one embodiment, the first and/or second gamete is selected where it has the wild type amino acid sequence of PLCD4 (SEQ ID No: 3).

In one embodiment of the twelfth aspect, the method further comprises the steps of: a) performing a method of the eighth aspect of the invention for the first and/or second gamete; and, b) selecting the first and/or second gamete where it is identified not to have an amino acid variation which disrupts PLCD4.

In a thirteenth aspect, the invention provides a method of breeding a non-human animal, the method comprising at least the step of selecting a non-human embryo that has been identified not to have an amino acid variation which disrupts PLCD4. A selected embryo is used to breed an animal. In one embodiment, the method further comprises transferring one or more embryo to a gestational carrier.

In one embodiment of the thirteenth aspect, the embryo is selected where it does not have an amino acid variation at a position corresponding to position 326 of PLCD4.

In one embodiment of the thirteenth aspect, the embryo is selected where it does not have the biological marker 326Thr.

In one embodiment of the thirteenth aspect, the embryo is selected where it has the biological marker 326Ala. In one embodiment, the embryo is selected where it has the wild type amino acid sequence of PLCD4 (SEQ ID No: 3).

In one embodiment of the thirteenth aspect, the method further comprises the steps of: a) performing a method of the eighth aspect of the invention for the embryo; and, b) selecting the embryo where it is identified not to have an amino acid variation which disrupts PLCD4.

In a fourteenth aspect, the invention provides a method of cloning a non-human animal, the method comprising at least the step of selecting a cell that has been identified not to have an amino acid variation which disrupts PLCD4. One or more selected cells are used to clone an animal.

In one embodiment of the fourteenth aspect, a cell is selected where it does not have an amino acid variation at a position corresponding to position 326 of PLCD4.

In one embodiment of the fourteenth aspect, a cell is selected where it does not have the biological marker 326Thr.

In one embodiment of the fourteenth aspect, a cell is selected where it has the biological marker 326Ala. In one embodiment, a cell is selected where it has the wild-type amino acid sequence of PLCD4 (SEQ ID No: 3).

In one embodiment of the fourteenth aspect, the method further comprises the steps of: a) performing a method of the eighth aspect of the invention for a cell; and, b) selecting a cell where it is identified not to have an amino acid variation which disrupts PLCD4.

In one embodiment of the eighth to fourteenth aspects, the methods involve analysing a PLCD4, precursor thereof, an isoform thereof and/or a fragment thereof to identify whether or not it includes two or more variations which disrupt PLCD4.

In one embodiment of the eighth to fourteenth aspects, the methods of the invention involve analysing a PLCD4, a precursor thereof, an isoform thereof and/or a fragment thereof to identify whether or not it includes a variation which disrupts PLCD4 alone or in combination with one or more other biological marker.

In one embodiment of the eighth to fourteenth aspects, the methods of the invention involve analysing a PLCD4, a precursor thereof, an isoform thereof and/or a fragment thereof to identify whether or not it includes an amino acid variation at a position corresponding to position 326 of PLCD4 alone or in combination with one or more other biological marker.

In one embodiment of the eighth to fourteenth aspects, the methods of the invention involve analysing a PLCD4, a precursor thereof, an isoform thereof and/or a fragment thereof to identify whether or not it includes the biological marker 326Thr alone or in combination with one or more other biological marker.

In a fifteenth aspect, the invention provides a method for identifying whether or not a non-human animal, cell or embryo carries a biological marker linked to a deleterious effect on productivity and/or worth of an animal, the method comprising at least the step of observing the level and/or activity of a PLCD4, a precursor thereof, an isoform thereof, a fragment thereof, and/or a nucleic acid encoding any one or more thereof of an animal, cell or embryo.

In one embodiment of the fifteenth aspect, the method comprises comparing the level and/or activity of PLCD4, a precursor thereof, an isoform thereof, a fragment thereof, and/or a nucleic acid encoding any one or more thereof against a standard.

In one embodiment, the method of the fifteenth aspect is used for selecting or rejecting a non-human animal, cell or embryo and/or estimating the worth of a non-human animal and/or its offspring.

In one embodiment, the method of the fifteenth aspect is used to identify whether or not an animal is suitable for (or at least more or less suitable for) inclusion in a herd, production purposes and/or breeding purposes. In another embodiment, the method is used to identify whether or not a cell or embryo is suitable for (or at least more or less suitable for) producing an animal which is suitable (or at least more suitable for) inclusion in a herd, production purposes and/or breeding purposes.

In a sixteenth aspect, the invention provides a method for selecting or rejecting a non-human animal, cell or embryo, the method comprising selecting the non-human animal, cell or embryo where it has been identified not to carry a biological marker linked to a deleterious effect on productivity and/or worth of an animal according to a method of the fifteenth aspect.

In one embodiment of the sixteenth aspect, the invention provides a method for selecting or rejecting a non-human animal, cell or embryo the method comprising at least the step of observing the level and/or activity of a PLCD4, a precursor thereof, an isoform thereof, a fragment thereof, and/or a nucleic acid encoding any one or more thereof.

In one embodiment of the sixteenth aspect, the method comprises comparing the level and/or activity of PLCD4, a precursor thereof, an isoform thereof, a fragment thereof, and/or a nucleic acid encoding any one or more thereof against a standard.

In one embodiment of the sixteenth aspect, the method comprises at least the steps of: a) performing a method of the fifteenth aspect; and, b) selecting or rejecting the non-human animal, cell or embryo using the results of step a).

In one embodiment of the sixteenth aspect, an animal, cell or embryo is selected if it does not carry a biological marker linked to a deleterious effect on productivity and/or worth. In one embodiment, an animal, cell or embryo is rejected if it does carry a biological marker linked to a deleterious effect on productivity and/or worth of an animal.

In one embodiment of the sixteenth aspect, the animal, cell or embryo is selected where the level and/or activity of PLCD4, a precursor thereof, an isoform thereof, a fragment thereof, and/or a nucleic acid encoding any one or more thereof is indicative of an animal(s) which is/are known not to carry a biological marker linked to a deleterious effect on productivity and/or worth of an animal. In one embodiment, an animal is rejected if the level and/or activity of PLCD4, a precursor thereof, an isoform thereof, a fragment thereof, and/or a nucleic acid encoding any one or more thereof is indicative of an animal(s) which is/are known to carry a biological marker linked to a deleterious effect on productivity and/or worth of an animal.

In one embodiment of the sixteenth aspect, the method is performed for the purpose of selecting or rejecting a non-human animal for milking purposes. In one embodiment, the method is performed for the purpose of selecting or rejecting a non-human animal for beef farming. In another embodiment, the method is performed for the purpose of selecting or rejecting a non-human animal for breeding purposes. In one embodiment, the method is performed for the purpose of selecting or rejecting a non-human animal for inclusion in a herd.

In other embodiments of the sixteenth aspect, the method is performed for the purpose of selecting or rejecting a non-human cell or embryo for use in cloning a non-human animal and/or breeding a non-human animal. In one embodiment, breeding an animal may involve IVF.

In a seventeenth aspect, the invention provides a method for estimating the worth of a non-human animal and/or its offspring, the method comprising at least the steps of: a) performing a method of the fifteenth aspect; and, b) estimating the worth of the animal and/or its offspring using the results of step a). For example, the method may comprise observing the level and/or activity of a PLCD4, a precursor thereof an isoform thereof, a fragment thereof, and/or a nucleic acid encoding any one or more thereof.

In one embodiment of the seventeenth aspect, the method comprises comparing the level and/or activity of PLCD4, a precursor thereof, an isoform thereof, a fragment thereof, and/or a nucleic acid encoding any one or more thereof against a standard.

In an eighteenth aspect, the invention provides a method for breeding a non-human animal, the method comprising at least the step of selecting a first non-human animal that has been identified not to carry a biological marker linked to a deleterious effect on productivity and/or worth of an animal and mating said first animal with a second non-human animal.

In one embodiment of the eighteenth aspect, the method further comprises selecting a second non-human animal that has been identified not to carry a biological marker linked to a deleterious effect on productivity and/or worth of an animal.

In one embodiment of the eighteenth aspect, the invention provides a method for breeding a non-human animal, the method comprising at least the step of selecting a first non-human animal where it has a level and/or activity of PLCD4, a precursor thereof, an isoform thereof, a fragment thereof, and/or a nucleic acid encoding any one or more thereof which is indicative of an animal(s) which is/are known not to carry a biological marker linked to a deleterious effect on productivity and/or worth and mating said first animal with a second non-human animal.

In one embodiment of the eighteenth aspect, the method further comprises selecting a second non-human animal where it has a level and/or activity of PLCD4, a precursor thereof, an isoform thereof a fragment thereof, and/or a nucleic acid encoding any one or more thereof which is indicative of an animal(s) which is/are known not to carry a biological marker linked to a deleterious effect on productivity and/or worth of an animal.

In one embodiment of the eighteenth aspect, the method further comprises the steps of: a) performing a method of the fifteenth aspect of the invention for the first and/or second non-human animal; and b) selecting the first and/or second non-human animal where it is identified not to have a level and/or activity of PLCD4, a precursor thereof, an isoform thereof, a fragment thereof, and/or a nucleic acid encoding any one or more thereof which is indicative of an animal(s) which is/are known not to carry a biological marker linked to a deleterious effect on productivity and/or worth of an animal.

In a nineteenth aspect, the invention provides a method for breeding a non-human animal, the method comprising at least the step of selecting a first gamete that has been identified not to carry a biological marker linked to a deleterious effect on productivity and/or worth of an animal and fusing said first gamete with a second gamete to form a zygote.

In one embodiment of the nineteenth aspect, the method further comprises selecting the second gamete that has been identified not to carry a biological marker linked to a deleterious effect on productivity and/or worth of an animal.

In one embodiment of the nineteenth aspect, the invention provides a method for breeding a non-human animal, the method comprising at least the step of selecting a first gamete that has been identified to have a level and/or activity of PLCD4, a precursor thereof, an isoform thereof, a fragment thereof, and/or a nucleic acid encoding any one or more thereof is indicative of an animal(s) which is/are known not to carry a biological marker linked to a deleterious effect on productivity and/or worth of an animal and fusing said first gamete with a second gamete to form a zygote.

In one embodiment of the nineteenth aspect, the method further comprises selecting the second gamete where it has been identified to have a level and/or activity of PLCD4, a precursor thereof, an isoform thereof, a fragment thereof, and/or a nucleic acid encoding any one or more thereof is indicative of an animal(s) which is/are known not to carry a biological marker linked to a deleterious effect on productivity and/or worth of an animal.

In one embodiment of the nineteenth aspect, the method further comprises the steps of: a) performing a method of the fifteenth aspect of the invention for the first and/or second gamete; and b) selecting the first and/or second gamete where it is identified not to have a level and/or activity of PLCD4, a precursor thereof an isoform thereof, a fragment thereof, and/or a nucleic acid encoding any one or more thereof which is indicative of an animal(s) which is/are known not to carry a biological marker linked to a deleterious effect on productivity and/or worth of an animal.

In a twentieth aspect, the invention provides a method for breeding a non-human animal, the method comprising at least the step of selecting a non-human embryo that has been identified not to carry a biological marker linked to a deleterious effect on productivity and/or worth of an animal.

In one embodiment of the twentieth aspect, the invention provides a method of breeding a non-human animal, the method comprising at least the step of selecting a non-human embryo that has been identified to have a level and/or activity of PLCD4, a precursor thereof, an isoform thereof, a fragment thereof, and/or a nucleic acid encoding any one or more thereof is indicative of an animal(s) which is/are known not to carry a biological marker linked to a deleterious effect on productivity and/or worth.

A selected embryo is used to breed an animal. In one embodiment, the method further comprises transferring one or more embryo to a gestational carrier.

In one embodiment of the twentieth aspect, the method further comprises the steps of: a) performing a method of the fifteenth aspect of the invention for the non-human embryo; and, b) selecting the embryo where it is identified not to have a level and/or activity of PLCD4, a precursor thereof, an isoform thereof, a fragment thereof, and/or a nucleic acid encoding any one or more thereof which is indicative of an animal(s) which is/are known not to carry a biological marker linked to a deleterious effect on productivity and/or worth of an animal.

In a twenty first aspect, the invention provides a method of cloning a non-human animal, the method comprising at least the step of selecting a non-human cell that has been identified not to carry a biological marker linked to a deleterious effect on productivity and/or worth of an animal.

In one embodiment of the twenty first aspect, the invention provides a method of cloning a non-human animal the method comprising at least the step of selecting a non-human cell that has been identified to have a level and/or activity of PLCD4, a precursor thereof, an isoform thereof, a fragment thereof, and/or a nucleic acid encoding any one or more thereof is indicative of an animal(s) which is/are known not to carry a biological marker linked to a deleterious effect on productivity and/or worth.

One or more selected cells are used to clone an animal.

In one embodiment of the twenty first aspect, the method further comprises the steps of: a) performing a method of the fifteenth aspect of the invention for the non-human cell; and, b) selecting the cell where it is identified not to have a level and/or activity of PLCD4, a precursor thereof, an isoform thereof, a fragment thereof, and/or a nucleic acid encoding any one or more thereof which is indicative of an animal(s) which is/are known not to carry a biological marker linked to a deleterious effect on productivity and/or worth of an animal.

In one embodiment of the fifteenth to twenty first aspects, the methods comprise the steps of:
a) observing the level and/or activity of a PLCD4, a precursor thereof, an isoform thereof, a fragment thereof, and/or a nucleic acid encoding one or more thereof in an animal, cell or embryo; and,
b) comparing the level and/or activity of the PLCD4, a precursor thereof, an isoform thereof, a fragment thereof, and/or a nucleic acid encoding one or more thereof against a standard.

In one embodiment of the fifteenth to twenty first aspects, the standard comprises a level and/or level of activity of PLCD4, a precursor thereof, an isoform thereof, a fragment thereof, and/or a nucleic acid encoding any one or more thereof which is associated with an animal or animals which is/are known not to carry a biological marker linked to a deleterious effect on productivity and/or worth of an animal. In one embodiment, an animal, cell or embryo is identified to carry a biological marker linked to a deleterious effect on productivity and/or worth of an animal where it has a lower level and/or level of activity of PLCD4, a precursor thereof, an isoform thereof, a fragment thereof and/or a nucleic acid encoding any one or more thereof compared to the standard. In one embodiment of the sixteenth or eighteenth to twenty first aspects, an animal, cell or embryo is rejected if it has a lower level and/or level of activity of PLCD4, a precursor thereof, an isoform thereof, a fragment thereof, and/or a nucleic acid encoding any one or more thereof compared to the standard. In one embodiment, an animal, cell or embryo is identified not to carry a biological marker linked to a deleterious effect on productivity and/or worth of an animal where it has substantially the same and/or a higher level of PLCD4, a precursor thereof, an isoform thereof, a fragment thereof, and/or a nucleic acid encoding any one or more thereof compared to the standard. In one embodiment of the sixteenth or eighteenth to twenty first aspects, an animal, cell or embryo is selected if it has substantially the same or a higher level and/or level of activity of PLCD4, a precursor thereof, an isoform thereof, a fragment thereof, and/or a nucleic acid encoding any one or more thereof compared to the standard.

In one embodiment of the fifteenth to twenty first aspects, the standard comprises a level and/or level of activity of PLCD4, a precursor thereof, an isoform thereof, a fragment thereof, and/or a nucleic acid encoding any one or more thereof which is associated with an animal or animals which is/are known to carry a biological marker linked to a deleterious effect on productivity and/or worth of an animal. In one embodiment, an animal, cell or embryo is identified not to carry a biological marker linked to a deleterious effect in productivity and/or worth where it has a higher level and/or level of activity of PLCD4, a precursor thereof, an isoform thereof, a fragment thereof, and/or a nucleic acid encoding any one or more thereof compared to the standard. In one embodiment, an animal, cell or embryo is identified to carry a biological marker linked to a deleterious effect in productivity and/or worth of an animal where it has substantially the same or a lower level and/or level of activity of PLCD4, a precursor thereof, an isoform thereof, a fragment thereof, and/or a nucleic acid encoding any one or more thereof compared to the standard. In one embodiment of the sixteenth or eighteenth to twenty first aspects, an animal, cell or embryo is selected if it has a higher level and/or level of activity of PLCD4, a precursor thereof, an isoform thereof, a fragment thereof, and/or a nucleic acid encoding any one or more thereof compared to the standard. In one embodiment of the sixteenth, or eighteenth to twenty first aspects, an animal, cell or embryo is rejected if it has substantially the same or a lower level and/or level of activity of PLCD4, a precursor thereof, an isoform thereof, a fragment thereof, and/or a nucleic acid encoding any one or more thereof compared to the standard.

In one embodiment of the fifteenth to twenty first aspects, the level of PLCD4, a precursor thereof, an isoform thereof, a fragment thereof, and/or a nucleic acid encoding any one or more thereof is the level of expression thereof. In another embodiment, the method involves observing the level of activity of PLCD4, a precursor thereof, an isoform thereof, and/or a fragment thereof.

In one embodiment of the fifteenth to twenty first aspects, the methods comprise observing the level and/or activity of a combination of two or more PLCD4, a precursor thereof, an isoform thereof, a fragment thereof, and/or a nucleic acid encoding any one or more thereof

In one embodiment of the fifteenth to twenty first aspects, the level and/or activity of PLCD4, a precursor thereof an isoform thereof, a fragment thereof, and/or a nucleic acid encoding any one or more thereof is observed alone or in combination with one or more other biological markers.

In one embodiment, the methods of the first to twenty first and thirty sixth and thirty seventh aspects may further involve analysing one or more additional biological markers. In one embodiment, the one or more biological markers is one or more genetic markers.

In one embodiment, the methods of the first to twenty first and thirty sixth and thirty seventh aspects of the invention may further involve taking a sample from an animal.

In one embodiment of the first to twenty first and thirty sixth and thirty seventh aspects of the invention, the absence of a variation which disrupts PLCD4, a genetic variation which disrupts the PLCD4 gene and/or a genetic marker in linkage disequilibrium therewith identifies that the animal, cell, gamete or embryo is suitable for use (or at least more suitable for use than an animal, cell, gamete or embryo that has a variation which disrupts PLCD4, a genetic variation which disrupts the PLCD4 gene and/or a genetic marker in linkage disequilibrium therewith, or at least more suitable than the same animal, cell, gamete or embryo if it had a variation which disrupts PLCD4, a genetic variation which disrupts the PLCD4 gene and/or marker in linkage disequilibrium therewith) for breeding and cloning purposes or that the animal is suitable for (or at least more suitable for as beforementioned) production purposes, breeding purposes and/or inclusion in a herd. In certain embodiments, an animal, cell, embryo or gamete is selected if it has the wild-type allele, wild-type nucleic acid sequence encoding PLCD4 or wild-type amino acid sequence.

In one embodiment of the first to twenty first and thirty sixth and thirty seventh aspects of the invention, the presence of a variation which disrupts PLCD4, a genetic variation which disrupts the PLCD4 gene and/or a genetic marker in linkage disequilibrium therewith identifies that the animal, cell, gamete or embryo is not suitable for use (or at least less suitable for use than an animal, cell, gamete or embryo that has a variation which disrupts PLCD4, a genetic variation which disrupts the PLCD4 gene and/or a genetic marker in linkage disequilibrium therewith, or at least less suitable than the same animal, cell, gamete or embryo if it had a variation which disrupts PLCD4, a genetic variation which disrupts the PLCD4 gene and/or marker in linkage disequilibrium therewith) for breeding and cloning purposes or that the animal is not suitable for (or at least less suitable for as beforementioned) production purposes, breeding purposes and/or inclusion in a herd.

In one embodiment of the first to twenty first and thirty sixth and thirty seventh aspects, where an animal is identified to carry a biological marker linked to a deleterious effect on productivity and/or worth of an animal it is identified not to be suitable (or at least to be less suitable than an animal or animals that do not have a biological marker linked to a deleterious effect on productivity and/or worth of an animal, or at least less suitable than the same animal if it did not carry a biological marker linked to a deleterious effect on productivity and/or worth of an animal) for inclusion in a herd, for production and/or breeding purposes. In one embodiment, where a cell or embryo is identified to carry a biological marker linked to a deleterious effect on productivity and/or worth of an animal it is identified not to be suitable (or at least less suitable than a cell or embryo that does not carry a biological marker linked to a deleterious effect on productivity and/or worth of an animal, or at least to be less suitable than the same cell or embryo if it did not carry a biological marker linked to a deleterious effect on productivity and/or worth of an animal) to produce an animal which is suitable for inclusion in a herd, for production and/or breeding purposes. In one embodiment, where an animal is identified not to carry a biological marker linked to a deleterious effect on productivity and/or worth of an animal it is identified to be suitable (or at least to be more suitable than an animal or animals that do carry a biological marker linked to a deleterious effect on productivity and/or worth of an animal, or at least more suitable than the same animal if it did carry a biological marker linked to a deleterious effect on productivity and/or worth of an animal) for inclusion in a herd, for production and/or breeding purposes. In one embodiment, where a cell or embryo is identified not to carry a biological marker linked to a deleterious effect on productivity and/or worth of an animal it is identified to be suitable (or at least to be more suitable than a cell or embryo that does carry a biological marker linked to a deleterious effect on productivity and/or worth of an animal or to be more suitable than the same cell or embryo if it did carry a biological marker linked to a deleterious effect on productivity and/or worth of an animal) to produce an animal which is suitable for production and/or breeding purposes.

In one embodiment of the first to twenty first and thirty sixth and thirty seventh aspects, where an animal is identified to be homozygous for a genetic marker linked to a deleterious effect on productivity and/or worth of an animal it is identified not to be suitable (or at least less suitable than an animal or animals that are not homozygous for a genetic marker linked to a deleterious effect on productivity and/or worth, or at least to be less suitable than the same animal if it was not homozygous for a genetic marker linked to a deleterious effect on productivity and/or worth of an animal) for inclusion in a herd, for production and/or breeding purposes. In one embodiment, where a cell or embryo is identified to be homozygous for a genetic marker linked to a deleterious effect on productivity and/or worth of an animal it is identified not to be suitable (or at least to be less suitable than a cell or embryo that is not homozygous for a genetic marker linked to a deleterious effect on productivity and/or worth of an animal, or at least less suitable than the same cell or embryo if it was not homozygous for a genetic marker linked to a deleterious effect on productivity and/or worth of an animal) to produce an animal which is suitable for inclusion in a herd, for production and/or breeding purposes. In one embodiment, where an animal is identified to be heterozygous for a genetic marker linked to a deleterious effect on productivity and/or worth of it is identified not to be suitable (or at least to be less suitable than an animal or animals that do not carry a genetic marker linked to a deleterious effect on productivity and/or worth of an animal, or at least to be less suitable than the same animal if it did not carry a genetic marker linked to a deleterious effect on productivity and/or worth of an animal) for inclusion in a herd, for production and/or breeding purposes. In one embodiment, where a cell or embryo is identified to be heterozygous for a genetic marker linked to a deleterious effect on productivity and/or worth of an animal it is identified not to be suitable (or at least to be less suitable than a cell or embryo that does not carry a genetic marker linked to a deleterious effect on productivity and/or worth of an animal, or at least to be less suitable than the same cell or embryo if it did not carry a genetic marker linked to a deleterious effect on productivity and/or worth of an animal) to produce an animal which is suitable for inclusion in a herd, for production and/or breeding purposes.

In a twenty second aspect, the invention provides a method of forming a herd of non-human animals, the method comprising selecting an animal where it has been identified not to carry a biological marker linked to a deleterious effect on productivity, worth of an animal and/or one or more related trait as described herein and forming a herd of selected animals.

In one embodiment of the twenty second aspect, the invention provides a method of forming a herd of non-human animals, the method comprising at least the steps of: a) selecting or rejecting a non-human animal based on the results of a method according to any one or more of the first, second, third, eighth, ninth, tenth, fifteenth, sixteenth, seventeenth, thirty sixth, thirty eighth or thirty ninth aspects of the invention; and, b) forming a herd of selected animals.

In one embodiment of the twenty second aspect, the invention provides a method of forming a herd of non-human animals, the method comprising at least the steps of:
a. performing a method of any one or more of the first, second, third, eighth, ninth, tenth, fifteenth, sixteenth, seventeenth, thirty sixth, thirty eighth or thirty ninth aspects of the invention;
b. selecting or rejecting an animal based on the results of step a.; and,
c. forming a herd of selected animals.

In one embodiment of the twenty second aspect, an animal is rejected if it has a biological marker linked to a deleterious effect on productivity and/or worth of an animal. In one embodiment, an animal is selected if it does not have a biological marker linked to a deleterious effect on productivity and/or worth of an animal.

In certain embodiments of the twenty second aspect, an animal is rejected if it includes one or more of:
a genetic variation which disrupts the PLCD4 gene (for example, a genetic variation at a position corresponding to position 107313998 of chromosome 2 of Bos Taurus, including, for example, 107313998A) and/or a genetic marker in linkage disequilibrium therewith;
an amino acid variation which disrupts PLCD4 (for example, a variation at a position corresponding to position 326 of PLCD4, including, for example, 326Thr); and/or,
a level or activity of PLCD4, a precursor thereof, an isoform thereof, a fragment thereof and/or a nucleic acid encoding any one or more thereof which is indicative of an animal or animals who is/are known to carry a biological marker linked to a deleterious effect on productivity and/or worth of an animal.

In certain embodiments of the twenty second aspect, an animal is selected if it:
does not have a genetic variation which disrupts the PLCD4 gene (for example, a variation at a position corresponding to position 107313998 on chromosome 2 of Bos taurus) and/or a genetic marker in linkage disequilibrium therewith;
has a nucleic acid encoding PLCD4 that has the wild-type allele or sequence (for example 107313998G or SEQ ID No: 1 and/or a genetic marker in linkage disequilibrium therewith;
does not have an amino acid variation which disrupts PLCD4 (for example, a variation at position 326);
has the wild-type amino acid sequence of PLCD4 (for example, Ala at a position corresponding to position 326 or SEQ ID No. 3; and/or,
has a level or activity of PLCD4, a precursor thereof, an isoform thereof, a fragment thereof and/or a nucleic acid encoding any one or more thereof which is indicative of an animal or animals who is/are known not to carry a biological marker linked to a deleterious effect on productivity and/or worth of an animal.

In a twenty third aspect, the invention provides a method for generating an animal, the method comprising at least the step of introducing a genetic alteration to the PLCD4 gene of a cell used to generate the animal.

In one embodiment of the twenty third aspect, the genetic alteration introduced to the PLCD4 gene corrects a variation in the PLCD4 gene which is linked to a deleterious effect on productivity and/or worth of an animal. In one embodiment, the variation in the PLCD4 gene which is linked to a deleterious effect on productivity and/or worth of an animal disrupts the PLCD4 gene.

In one embodiment of the twenty third aspect, the method is conducted for the purpose of generating (or at least increasing the likelihood of generating) an animal which does not carry a biological marker which is linked to a deleterious effect on productivity and/or worth of an animal. In one embodiment, the method is conducted for the purpose of generating (or at least increasing the likelihood of generating) an animal which is suitable for production and/or breeding purposes. In one embodiment, the method is conducted for the purpose of generating (or at least increasing the likelihood of generating) an animal having desirable worth and/or productivity.

In a twenty fourth aspect, the invention provides a method for generating a cell or embryo which is of use in a method for generating an animal, the method comprising at least the step of introducing a genetic alteration to the PLCD4 gene of the cell or embryo.

In one embodiment of the twenty fourth aspect, the genetic alteration introduced to the PLCD4 gene corrects a variation in the PLCD4 gene which is linked to a deleterious effect on productivity and/or worth of an animal. In one embodiment, the variation in the PLCD4 gene which is linked to a deleterious effect on productivity and/or worth of an animal disrupts the PLCD4 gene.

In one embodiment of the twenty fourth aspect, the method is conducted for the purpose of generating (or at least increasing the likelihood of generating) a cell or embryo of use in generating an animal which does not carry a biological marker which is linked to a deleterious effect on productivity and/or worth of an animal. In one embodiment, the method is conducted for the purpose of generating (or at least increasing the likelihood of generating) a cell or embryo of use in generating an animal which is suitable for production and/or breeding purposes. In one embodiment, the method is conducted for the purpose of generating (or at least increasing the likelihood of generating) a cell or embryo which may be used to generate an animal having desirable worth and/or productivity

In one embodiment of the twenty third and twenty fourth aspects, the method for generating the animal involves IVF. In one embodiment, the method for generating the animal is a cloning method.

In one embodiment of the twenty third and twenty fourth aspects, a genetic alteration which is introduced includes a genetic alteration located within a region defined by nucleotides corresponding to positions 107313997 to 107314141 of chromosome 2 of Bos taurus. In one embodiment, the genetic alteration which is introduced includes a genetic alteration at a position corresponding to position 107313998 on chromosome 2 of Bos taurus. In one embodiment, the genetic alteration which is introduced includes a substitution of an A at the position corresponding to position 107313998 on chromosome 2 of Bos taurus. In one embodiment, the A is substituted with a G. In one embodiment, the genetic alteration which is introduced results in the PLCD4 gene having the sequence of SEQ ID No: 1.

In one embodiment of the twenty third and twenty fourth aspects, the cell is chosen from a gamete or a zygote. In one embodiment, the cell is a somatic cell or a cell from a cell line.

In one embodiment, where a method of the twenty third or fourth aspects uses cloning, it may further comprise the step of selecting or rejecting an animal, cell or embryo using a method of any one or more of the methods of the first, second, third, eighth, ninth, tenth, fifteenth, sixteenth or seventeenth aspects of the invention. This step may be conducted prior to and/or after the step of introducing a genetic alteration to the PLCD4 gene to select a starting cell and/or to confirm the alteration is present in a resulting cell, embryo and/or animal.

In one embodiment, where a method of the twenty third or fourth aspects uses IVF, it may further comprise the step of selecting or rejecting an animal, cell or embryo using a method of any one or more of the methods of the first, second, third, eighth, ninth, tenth, fifteenth, sixteenth or seventeenth aspects of the invention. This step may be conducted prior to and/or after the step of introducing a genetic alteration to the PLCD4 gene to select a starting cell and/or to confirm the alteration is present in a resulting cell, embryo and/or animal.

In a twenty fifth aspect, the invention provides a cell capable of use in a method of generating a non-human animal, wherein a genetic alteration has been introduced to a PLCD4 gene of the cell, and the genetic alteration corrects a variation in the PLCD4 gene which is linked to a deleterious effect on productivity and/or worth of an animal.

In a twenty sixth aspect, the invention provides an embryo capable of use in a method of generating a non-human animal, wherein a genetic alteration has been introduced to a PLCD4 gene of the embryo, and the genetic alteration corrects a variation in the PLCD4 gene which is linked to a deleterious effect on productivity and/or worth of an animal.

In a twenty seventh aspect, the invention provides a method for generating a non-human animal, the method comprising using a cell or embryo to which a genetic alteration to a PLCD4 gene has been introduced to generate the non-human animal, wherein the genetic alteration corrects a variation in the PLCD4 gene which is linked to a deleterious effect on productivity and/or worth of an animal.

In a twenty eighth aspect, the invention provides a method for increasing the likelihood that a non-human animal will have a desirable worth and/or productivity, the method comprising using a cell or embryo to which a genetic alteration to a PLCD4 gene has been introduced to generate the non-human animal, wherein the genetic alteration corrects a variation in the PLCD4 gene which is linked to a deleterious effect on productivity and/or worth of an animal.

In a twenty ninth aspect, the invention provides a method for increasing the likelihood that a non-human animal is suitable for inclusion in a herd, for production and/or breeding purposes, the method comprising using a cell or embryo to which a genetic alteration to a PLCD4 gene has been introduced to generate the non-human animal, wherein the genetic alteration corrects a variation in the PLCD4 gene which is linked to a deleterious effect on productivity and/or worth of an animal.

In a thirtieth aspect, the invention provides a method for generating a non-human animal, the method comprising at least the step of introducing a genetic alteration to a PLCD4 gene of a cell or embryo and using the cell or embryo to generate a non-human animal, wherein the genetic alteration corrects a variation in the PLCD4 gene which is linked to a deleterious effect on productivity and/or worth of an animal.

In a thirty first aspect, the invention provides a method for increasing the likelihood that a non-human animal to be generated will have a desirable worth and/or productivity, the method comprising at least the step of introducing a genetic alteration to a PLCD4 gene of a cell or embryo and using the cell or embryo to generate a non-human animal, wherein the genetic alteration corrects a variation in the PLCD4 gene which is linked to a deleterious effect on productivity and/or worth of an animal.

In a thirty second aspect, the invention provides a method for increasing the likelihood that a non-human animal to be generated will be suitable for inclusion in a herd, for production and/or breeding purposes, the method comprising at least the step of introducing a genetic alteration to a PLCD4 gene of a cell or embryo and using the cell or embryo to generate a non-human animal, wherein the genetic alteration corrects a variation in the PLCD4 gene which is linked to a deleterious effect on productivity and/or worth of an animal.

In a thirty third aspect, the invention provides a method for generating an animal that will not carry a biological marker linked to a deleterious effect on productivity and/or worth of an animal, the method comprising using a cell or embryo to which a genetic alteration to a PLCD4 gene has been introduced to generate the non-human animal, wherein the genetic alteration corrects a variation in the PLCD4 gene which is linked to a deleterious effect on productivity and/or worth of an animal.

In thirty fourth aspect, the invention provides a method for increasing the likelihood that a non-human animal will not carry a biological marker linked to a deleterious effect on productivity and/or worth of an animal, the method comprising using a cell or embryo to which a genetic alteration to a PLCD4 gene has been introduced to generate the non-human animal, wherein the genetic alteration corrects a variation in the PLCD4 gene which is linked to a deleterious effect on productivity and/or worth of an animal.

In a thirty fifth aspect, the invention provides a method for increasing the likelihood that a non-human animal to be generated will not carry a biological marker linked to a deleterious effect on productivity and/or worth of an animal, the method comprising at least the step of introducing a genetic alteration to a PLCD4 gene of a cell or embryo and using the cell or embryo to generate a non-human animal, wherein the genetic alteration corrects a variation in the PLCD4 gene which is linked to a deleterious effect on productivity and/or worth of an animal.

In one embodiment of the twenty fifth to thirty fifth aspects, the variation in the PLCD4 gene which is linked to a deleterious effect on productivity and/or worth of an animal disrupts the PLCD4 gene.

In one embodiment of the twenty third to thirty fifth aspects, more than one genetic alteration is introduced into a cell or embryo. In one embodiment, one or more genetic alteration introduced to a cell or embryo is introduced to correct one or more variation in the PLCD4 gene which is linked to a deleterious effect on productivity and/or worth of an animal.

In a thirty sixth aspect, the invention provides a method to identify whether or not an animal is suitable for (or at least more or less suitable for) inclusion in a herd, production purposes and/or breeding purposes. In one embodiment, the method comprises analysing a nucleic acid of said animal to identify whether or not it includes a genetic variation which disrupts the PLCD4 gene and/or a genetic marker in linkage disequilibrium therewith. In another embodiment, the method comprises analysing a PLCD4, a precursor thereof, an isoform thereof and/or a fragment thereof of said animal to identify whether or not it includes a variation which disrupts PLCD4. In another embodiment, the method comprises observing the level and/or activity of a PLCD4, a precursor thereof, an isoform thereof a fragment thereof, and/or a nucleic acid encoding any one or more thereof of the animal. In one embodiment, where the animal is identified to have a variation which disrupts the PLCD4 gene or PLCD4 or to have a level or level of activity of PLCD4, a precursor thereof, an isoform thereof and/or a fragment thereof which is indicative of an animal(s) which is/are known to carry a biological marker linked to a deleterious effect on productivity and/or worth of an animal, the animal is identified not to be suitable for (or at least less suitable for) inclusion in a herd, production purposes and/or breeding purposes. In one embodiment, where the animal is identified not to have a variation which disrupts the PLCD4 gene or PLCD4 or to have a level or level of activity of PLCD4, a precursor thereof, an isoform thereof and/or a fragment thereof which is indicative of an animal(s) which is/are known not to carry a biological marker linked to a deleterious effect on productivity and/or worth of an animal, the animal is identified to be suitable for (or at least more suitable for) inclusion in a herd, production purposes and/or breeding purposes.

In a thirty seventh aspect, the invention provides a method to identify whether or not an embryo or cell is suitable for (or at least more or less suitable for) producing an animal which is suitable for inclusion in a herd, production purposes and/or breeding purposes. In one embodiment, the method comprises analysing a nucleic acid of said cell or embryo to identify whether or not it includes a genetic variation which disrupts the PLCD4 gene and/or a genetic marker in linkage disequilibrium therewith. In another embodiment, the method comprises analysing a PLCD4, a precursor thereof, an isoform thereof and/or a fragment thereof of said cell ore embryo to identify whether or not it includes a variation which disrupts PLCD4. In another embodiment, the method comprises observing the level and/or activity of a PLCD4, a precursor thereof, an isoform thereof, a fragment thereof, and/or a nucleic acid encoding any one or more thereof of the cell or embryo. In one embodiment, where the cell or embryo is identified to have a variation which disrupts the PLCD4 gene or PLCD4 or to have a level or level of activity of PLCD4, a precursor thereof, an isoform thereof and/or a fragment thereof which is indicative of an animal(s) which is/are known to carry a biological marker linked to a deleterious effect on productivity and/or worth of an animal, the cell or embryo is identified not to be suitable for (or at least less suitable for) producing an animal which is suitable for inclusion in a herd, production purposes and/or breeding purposes. In one embodiment, where the cell or embryo is identified not to have a variation which disrupts the PLCD4 gene or PLCD4 or to have a level or level of activity of PLCD4, a precursor thereof, an isoform thereof and/or a fragment thereof which is indicative of an animal(s) which is/are not known to carry a biological marker linked to a deleterious effect on productivity and/or worth of an animal, the cell or embryo is identified to be suitable for (or at least more suitable for) producing an animal which is suitable for inclusion in a herd, production purposes and/or breeding purposes.

In one embodiment of the thirty sixth and thirty seventh aspects, the methods comprise: a) performing method of the first, eighth or fifteenth aspects; and b) identifying whether or not an animal is suitable (or at least more or less suitable) for inclusion in a herd, production purposes and/or breeding purposes or whether or not a cell or embryo is suitable (or at least more or less suitable) for producing an animal which is suitable (or at least more or less suitable) for inclusion in a herd, production purposes and/or breeding purposes.

In a thirty eighth aspect, the invention provides a method for identifying whether or not a non-human animal, cell or embryo carries a biological marker linked to a deleterious effect on protein yield, milk yield, fat yield, liveweight, stature, body condition score, rump width, milking speed, capacity, dairy conformation, overall opinion and/or adaptability. In one embodiment, the method comprises analysing a nucleic acid of said animal, cell or embryo to identify whether or not it includes a genetic variation which disrupts the PLCD4 gene and/or a genetic marker in linkage disequilibrium therewith.

In another embodiment, the method comprises analysing a PLCD4, a precursor thereof, an isoform thereof and/or a fragment thereof of said animal, cell or embryo to identify whether or not it includes a variation which disrupts PLCD4. In another embodiment, the method comprises observing the level and/or activity of a PLCD4, a precursor thereof, an isoform thereof, a fragment thereof, and/or a nucleic acid encoding any one or more thereof of the animal, cell or embryo. In one embodiment, where the animal, cell or embryo includes a variation which disrupts the PLCD4 gene or PLCD4 or to have a level or level of activity of PLCD4, a precursor thereof, an isoform thereof and/or a fragment thereof which is indicative of an animal(s) which is/are known to carry a biological marker linked to a deleterious effect on protein yield, milk yield, fat yield, liveweight, stature, body condition score, rump width, milking speed, capacity, dairy conformation, overall opinion and/or adaptability, it is identified to carry a biological marker linked to a deleterious effect on protein yield, milk yield, fat yield, liveweight, stature, body condition score, rump width, milking speed, capacity, dairy conformation, overall opinion and/or adaptability.

In one embodiment of the thirty eighth aspect, the method is for identifying whether or not a non-human animal, cell or embryo carries a biological marker linked to a deleterious effect on liveweight, protein yield, milk yield, and fat yield. In one embodiment, the method is for identifying whether or not a non-human animal, cell or embryo carries a biological marker linked to a deleterious effect on protein yield, milk yield, and fat yield. In one embodiment, the method is for identifying whether or not a non-human animal, cell or embryo carries a biological marker linked to a deleterious effect on liveweight. In one embodiment, the method is for identifying whether or not a non-human animal, cell or embryo carries a biological marker linked to a deleterious effect on liveweight, stature, body condition score, rump width, milking speed, capacity, dairy conformation, overall opinion and adaptability. In one embodiment, the method is for identifying whether or not a non-human animal, cell or embryo carries a biological marker linked to a deleterious effect on stature, body condition score, rump width, milking speed, capacity, dairy conformation, overall opinion and adaptability.

In one embodiment of the thirty eighth aspect, the genetic variation which disrupts the PLCD4 gene and/or a genetic marker in linkage disequilibrium therewith, results in a decrease in the level and/or activity of PLCD4. In one embodiment of the thirty eighth aspect, the genetic variation which disrupts the PLCD4 gene and/or a genetic marker in linkage disequilibrium therewith, is a missense variant. In one embodiment of the thirty eighth aspect, the genetic variation which disrupts the PLCD4 gene is located within a region defined by nucleotides corresponding to positions 107313997 to 107314141 of chromosome 2 of Bos Taurus (which defines exon 8). In one embodiment of the thirty eighth aspect, the genetic variation which disrupts the PLCD4 gene is located at position 107313998 of chromosome 2 of Bos Taurus (which defines exon 8).

In a thirty ninth aspect, the invention provides a method for selecting or rejecting a non-human animal, cell or embryo, the method comprising selecting the non-human animal, cell or embryo where it has been determined not to carry a biological marker linked to a deleterious effect on protein yield, milk yield, fat yield, liveweight, stature, body condition score, rump width, milking speed, capacity, dairy conformation, overall opinion and/or adaptability. In one embodiment, the method comprises at least the steps of: a) performing a method of the thirty eighth aspect; and b) selecting or rejecting the non-human animal, cell or embryo using the results of step a).

In other aspects, the invention provides:
An animal selected by a method of any one or more of the second, ninth, sixteenth and thirty ninth aspects;
A cell or embryo selected by a method of any one or more of the second, ninth sixteenth and thirty ninth aspects;
Offspring or an animal produced by a method of any one or more of the fourth, fifth, sixth, seventh, eleventh, twelfth, thirteenth, fourteenth, eighteenth, nineteenth, twentieth, and twenty first, aspects;
A herd formed using a method of the twenty second aspect of the invention;
An animal generated by a method of any one or more of the twenty third, and twenty seventh to thirty fifth aspects;
A cell generated by a method of the twenty fourth aspect; and/or,
An embryo generated by a method of the twenty fourth aspect.

In other aspects, the invention provides:
Use of an animal selected according to a method of any one or more of the second, ninth sixteenth and thirty ninth aspects to breed or clone a non-human animal;
Use of a cell or embryo selected according to a method any one or more of the second, ninth sixteenth and thirty ninth aspects to breed or clone a non-human animal;
Use of an animal generated according to a method of any one or more of the twenty third and twenty seventh to thirty fifth aspects to breed or clone a non-human animal;
Use of a cell or embryo of the twenty fourth, twenty fifth, and twenty sixth aspect to breed or clone a non-human animal;
Use of an animal selected according to a method of any one or more of the second, ninth sixteenth and thirty ninth aspects to form a herd;
Use of an animal generated according to a method of any one or more of the twenty third and twenty seventh to thirty fifth aspects to form a herd;
Use of the estimated worth of an animal, according to the third, tenth, and/or seventeenth aspects to select or reject an animal;
A method for selecting or rejecting an animal, the method comprising selecting and/or rejecting an animal based on the results of a method of the third, tenth and/or seventeenth aspects;
A method for selecting or rejecting an animal, the method comprising: a) performing a method of the third, tenth and/or seventeenth aspects; and b) selecting or rejecting an animal based on the results of a).
Use of the results of a method of the first, third, eighth, tenth, fifteenth, seventeenth and/or thirty eighth aspects to identify whether or not an animal is suitable for (or at least more or less suitable for) for inclusion in a herd, production purposes and/or breeding purposes.
A method to identify whether or not an animal is suitable for (or at least more or less suitable for) inclusion in a herd, for production purposes and/or breeding purposes, the method comprising predicting the animal's suitability based on the results of a method of the first, third, eighth, tenth, fifteenth and/or seventeenth aspects;
A method to identify whether or not an animal is suitable for (or at least more or less suitable for) inclusion in a herd, for production purposes and/or breeding purposes, the method comprising: a) performing a method of the first, third, eighth, tenth, fifteenth and/or seventeenth aspects; and, b) predicting the animal's suitability based on the results of a);
Use of the results of a method of the first, eighth, fifteenth and/or thirty eighth aspects to identify whether or not a cell or embryo is suitable for (or at least more or less suitable for) generating an animal which is suitable for inclusion in a herd, production purposes and/or breeding purposes.
A method to identify whether or not a cell or embryo is suitable for (or at least more or less suitable for) generating an animal which is suitable for inclusion in a herd, for production purposes and/or breeding purposes, the method comprising predicting the animal's suitability based on the results of a method of the first, third, eighth, tenth, fifteenth and/or seventeenth aspects;
A method to identify whether or not a cell or embryo is suitable for (or at least more or less suitable for) generating an animal which is suitable for inclusion in a herd, for production purposes and/or breeding purposes, the method comprising: a) performing a method of the first, third, eighth, tenth, fifteenth and/or seventeenth aspects; and, b) predicting the animal's suitability based on the results of a);
A method for estimating the worth of an animal, the method comprising the step of incorporating information on a biological marker of the invention of the animal in the worth calculation; and/or
A method for selecting or rejecting an animal cell or embryo, the method comprising the step of selecting or rejecting an animal, cell or embryo using information on a biological marker of the animal, cell or embryo.

In another aspect, the invention provides a method for identifying a genetic variation in the PLCD4 gene which is linked to a deleterious effect on productivity and/or worth of an animal. In one embodiment, the method comprises identifying a genetic variation in the PLCD4 gene which disrupts the gene. In one embodiment, where the genetic variation disrupts the PLCD4 gene, the variation is identified to be linked to a deleterious effect on productivity and/or worth of an animal. In one embodiment, the method further comprises identifying whether or not the alteration results in a decrease in the level and/or activity of PLCD4. In one embodiment, where the alteration results in a decrease in the level and/or activity of PLCD4 the genetic alteration is identified as being linked to a deleterious effect on productivity and/or worth of an animal. In one embodiment, the method further comprises determining whether or not the identified genetic variation in the PLCD4 gene is associated with an animal or animals known to have a variation which disrupts PLCD4 and/or less than desirable productivity and/or worth.

In another aspect, the invention provides a method for identifying an amino acid variation in PLCD4 which is linked to a deleterious effect on productivity and/or worth of an animal. In one embodiment, the method comprises identifying a variation in PLCD4 which disrupts PLCD4. In one embodiment, where the genetic variation disrupts PLCD4, the variation is identified to be linked to a deleterious effect on productivity and/or worth of an animal. In one embodiment, the method further comprises identifying whether or not the variation results in a decrease in the level and/or activity of PLCD4. In one embodiment, where the alteration results in a decrease in the level and/or activity of PLCD4 the variation is identified as being linked to a deleterious effect on productivity and/or worth of an animal. In one embodiment, the method further comprises determining whether or not the identified variation in PLCD4 is associated with an animal or animals known to have a variation which disrupts PLCD4 and/or less than desirable productivity and/or worth.

In one embodiment of any one or more of the aspects of the invention the non-human animal is, or the non-human cell or embryo is from, a production animal. In one embodiment of any one or more aspects of the invention, the production animal is a mammal. In one particular embodiment of any one or more aspects of the invention, the animal is, or the cell or embryo is from a ruminant. In another embodiment of any one or more aspects of the invention, the animal, cell or embryo is from the Bovidae family. In one embodiment of any one or more aspects of the invention, the animal, cell or embryo is bovine. In one particular embodiment of any one or more aspects of the invention, the bovine is Bos taurus or Bos indicus. In a particular embodiment of any one or more aspects of the invention, the bovine is chosen from the group consisting Jersey, Holstein-Friesian or crossbred dairy cattle. In one particular embodiment of any one or more aspects of the invention, the bovine is Holstein-Friesian.

In one embodiment of any one or more aspects of the invention, the deleterious effect on productivity and/or worth includes a deleterious effect on one or more of the following animal assessment parameters: milk yield; protein yield; and fat yield. In one embodiment of any one or more aspects of the invention, the deleterious effect on productivity and/or worth includes a deleterious effect on one or more of the following animal assessment parameters: liveweight; milk yield; protein yield; and fat yield. In one embodiment of any one or more aspects of the invention, the deleterious effect on productivity and/or worth includes a deleterious effect on one or more of the following animal assessment parameters: liveweight; stature; body condition score; rump width; milking speed; capacity; dairy conformation; overall opinion; and adaptability. In one embodiment of any one or more aspects of the invention, the deleterious effect on productivity and/or worth includes a deleterious effect on one or more of the following animal assessment parameters: stature; body condition score; rump width; milking speed; capacity; dairy conformation; overall opinion; and adaptability. In one embodiment of any one or more aspects of the invention, the deleterious effect on productivity and/or worth includes a deleterious effect on one or more of the following animal assessment parameters: liveweight; stature; body condition score; rump width; protein yield; milk yield; fat yield; milking speed; capacity; dairy conformation; overall opinion; and adaptability.

In one particular embodiment of any one or more aspects of the invention, the deleterious effect on productivity and/or worth includes a deleterious effect on all of the following animal assessment parameters: protein yield; milk yield; and, fat yield. In one particular embodiment of any one or more aspects of the invention, the deleterious effect on productivity and/or worth includes a deleterious effect on all of the following animal assessment parameters: liveweight; protein yield; milk yield; and, fat yield.

In another embodiment of any one or more aspects of the invention, the deleterious effect on productivity and/or worth includes a deleterious effect on all of the following animal assessment parameters: stature; body condition score; rump width; milking speed; capacity; dairy conformation; overall opinion; and adaptability. In another embodiment of any one or more aspects of the invention, the deleterious effect on productivity and/or worth includes a deleterious effect on all of the following animal assessment parameters: liveweight; stature; body condition score; rump width; milking speed; capacity; dairy conformation; overall opinion; and adaptability.

In another embodiment of any one or more aspects of the invention, the deleterious effect on productivity and/or worth includes a deleterious effect on all of the following animal assessment parameters: liveweight; stature; body condition score; rump width; protein yield; milk yield; fat yield; milking speed; capacity; dairy conformation; overall opinion; and adaptability.

In another aspect, the invention provides an isolated nucleic acid comprising the genetic marker 107313998A on chromosome 2 of Bos taurus and/or a genetic marker in linkage disequilibrium therewith. In one embodiment, the invention provides a nucleic acid comprising or consisting the sequence of SEQ ID No. 2 or a functionally equivalent variant thereof. In one embodiment, the invention provides an oligonucleotide or part of the nucleic acid comprising or consisting of the sequence of SEQ ID No. 2. In another aspect, the invention provides a nucleic acid which is complementary to or can hybridise to said nucleic acids, in one embodiment under stringent conditions.

In one embodiment, the nucleic acid is chosen from the group comprising DNA, cDNA and mRNA, including single- and double-stranded nucleic acids, for example.

In another aspect, the invention provides an isolated peptide encompassing a variation which disrupts PLCD4. In one embodiment, the peptide comprises an amino acid corresponding to an amino acid at position 326 of PLCD4 (for example, Thr at this position). In one embodiment, the peptide comprises or consists the sequence of SEQ ID No. 4. In one embodiment, the invention provides a fragment, isoform or precursor of the peptide comprising or consisting the sequence of SEQ ID No. 4 or a part of any one or more thereof or a functionally equivalent variant thereof.

In another aspect, the invention provides other isolated nucleic acids and/or peptides and/or proteins as may be described herein.

For the avoidance of doubt, where any of the aspects includes analysing a nucleic acid or analysing PLCD4, gene product or PLCD4 protein, a precursor thereof, an isoform thereof and/or a fragment thereof, or observing the level and/or activity of a PLCD4, this may be done in vitro.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1: shows a version of SEQ ID No. 1 showing 1000 bp of non-coding sequences both 5′ and 3′ of the first and last exons, though for brevity, all introns have been removed aside from the introns flanking the exon of interest (i.e. introns 7 and 8 that flank exon 8 encompassing the chr2:107313998 substitution). Intron-Exon boundaries for introns that have been removed are demarcated by ‘pipe’ symbols, and where relevant, exons are provided in uppercase, and intronic and intergenic sequences are provided in lowercase. Site of the G>A substitution is bracketed.

FIG. 2: shows a version of SEQ ID NO: 2 showing 1000 bp of non-coding sequences both 5′ and 3′ of the first and last exons, though for brevity, all introns have been removed aside from the introns flanking the exon of interest (i.e. introns 7 and 8 that flank exon 8 encompassing the chr2:107313998 substitution). Intron-Exon boundaries for introns that have been removed are demarcated by ‘pipe’ symbols, and where relevant, exons are provided in uppercase, and intronic and intergenic sequences are provided in lowercase. Exons are provided in uppercase, introns and intergenic sequence is provided in lowercase. Site of G>A substitution is bracketed.

FIG. 3: shows SEQ ID No. 3—protein sequence indicating the reference (wild type) form of PLCD4. NCBI Reference Sequence: NP_001039954.1 1-phosphatidylinositol 4,5-bisphosphate phosphodiesterase delta-4 [Bos taurus]. Site of Ala>Thr substitution is bracketed.

FIG. 4: shows SEQ ID No. 4—Protein sequence indicating the alternate (mutant) form of PLCD4. NCBI Reference Sequence: NP_001039954.1 1-phosphatidylinositol 4,5-bisphosphate phosphodiesterase delta-4 [Bos taurus]. Site of Ala>Thr substitution is bracketed.

FIG. 5: shows SEQ ID No. 5—Sequence representing the wild type gene mRNA transcript of PLCD4 (Refseq ID NM_001046489.2). The position of the nucleotide substitution at position 1218 identified by the inventors is bracketed.

FIG. 6: shows SEQ ID No. 6—Sequence representing the predicted mutant mRNA transcript of PLCD4 (based on Refseq ID NM_001046489.2). The site of the nucleotide substitution in the mutant transcript is bracketed. The position of the variant is nucleotide 1218.

FIG. 7: shows the multiple protein sequence alignment using a database of model organism peptides showing strong evolutionary conservation of the Ala residue at position 326 between Cow, Human, Mouse, Zebrafish, and Nematode: 1) Cow PLCD4—mutant; 2) Cow PLCD4; 3) Mouse PLCD4; 5) Zebrafish PLCD4; and, 6) Nematode PLCD4.

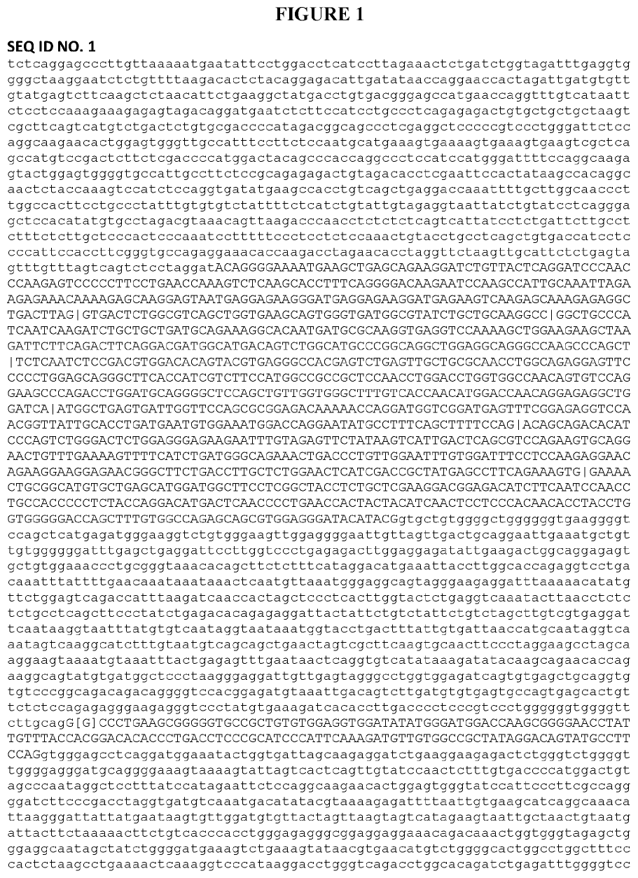

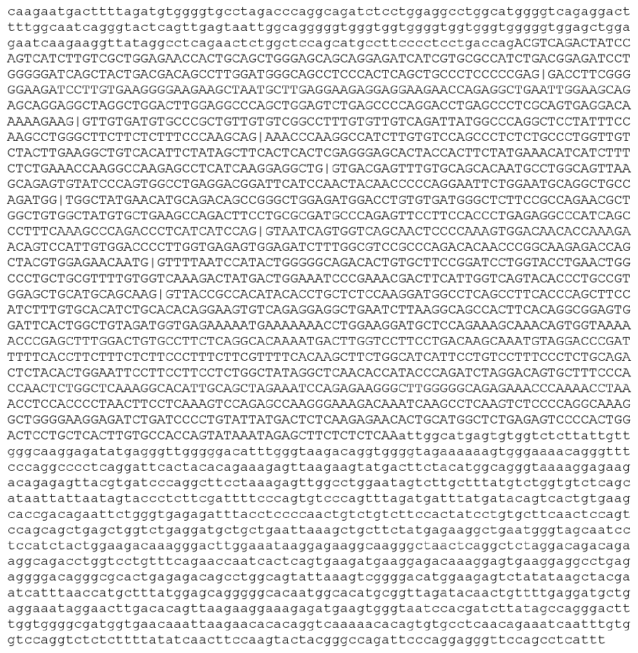

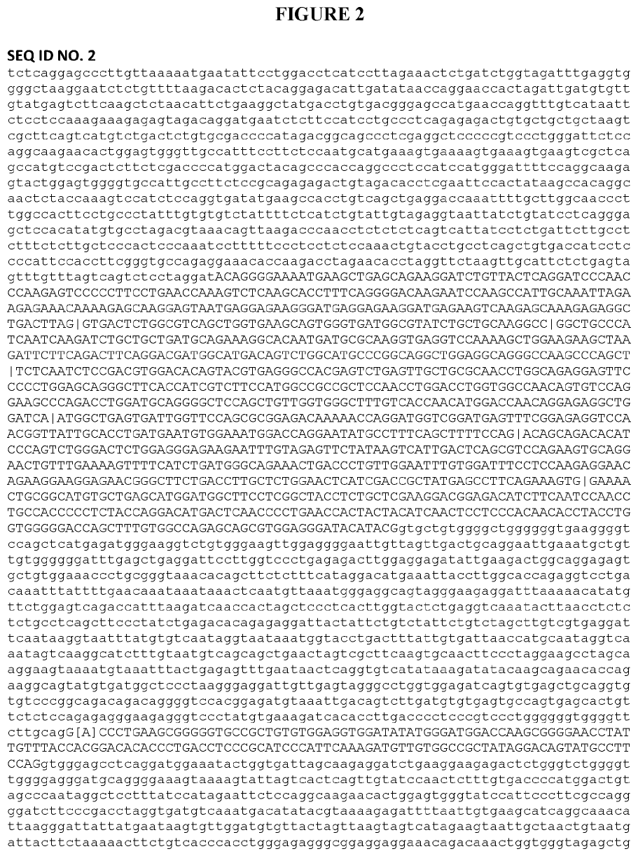

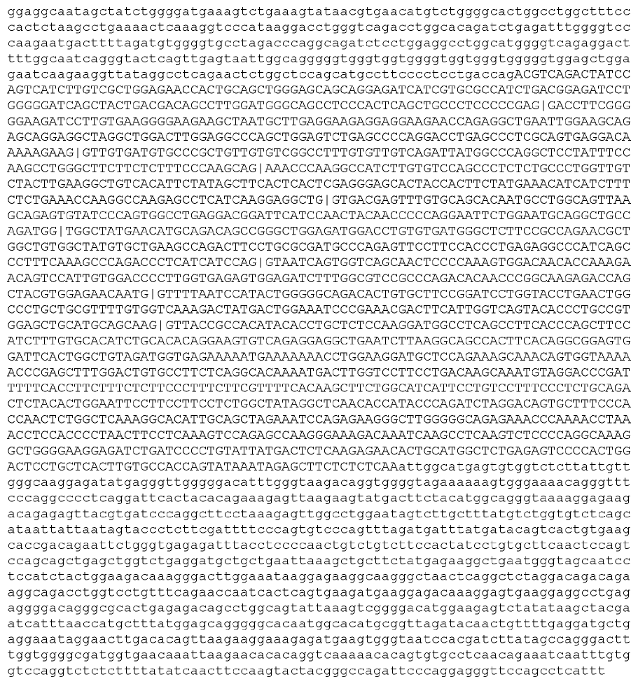

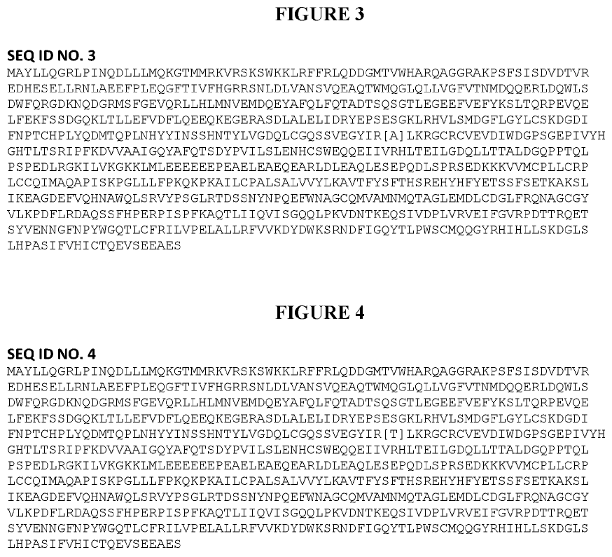

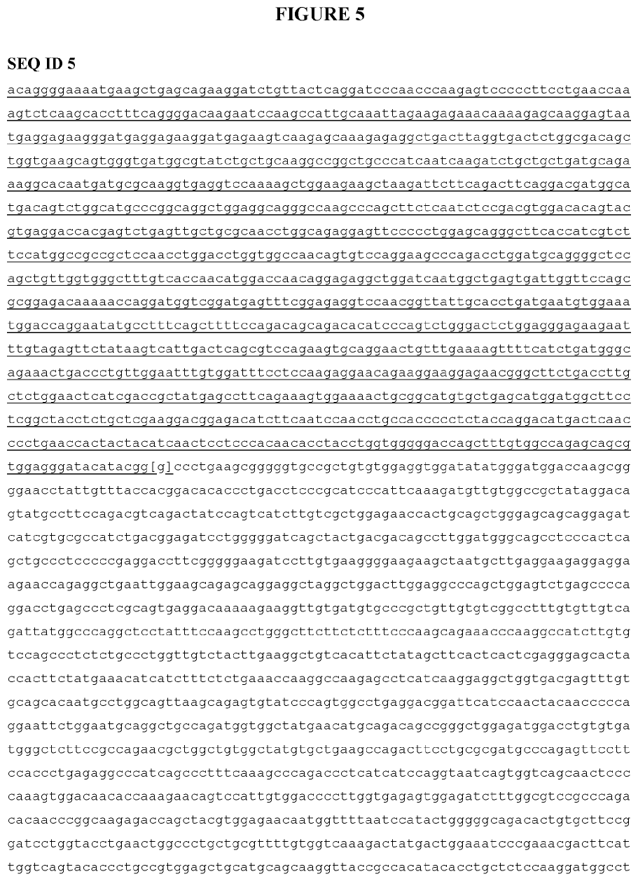

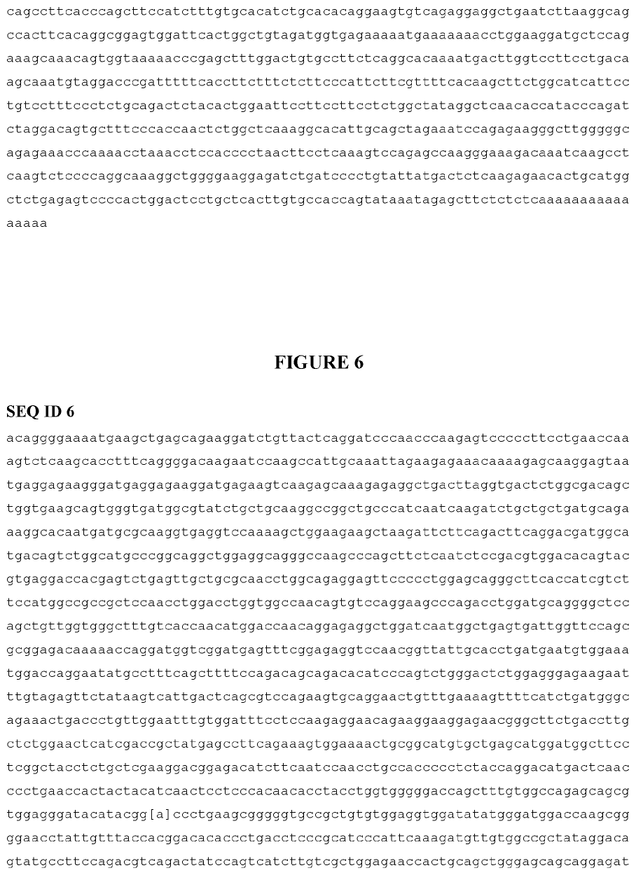

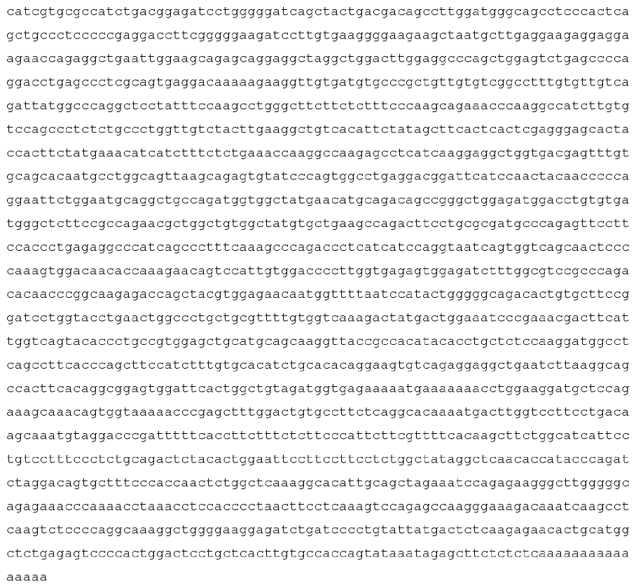

### PREFERRED EMBODIMENT(S)

The following is a description of the present invention, including preferred embodiments thereof, given in general terms. The invention is further elucidated from the disclosure given under the section “Examples” which provides, inter alia, data supporting the invention.

The inventors have identified a genetic variation in the PLCD4 (Phospholipase C delta 4) gene which has a deleterious effect on a number of traits representative of productivity and used in worth calculations for an animal, when in a homozygous state. The inventors contemplate that methods involving the analysis of nucleic acid to determine whether or not it includes such an alternation in PLCD4 can be used, inter alia, to identify whether or not an animal, cell or embryo carries a biological marker linked to a deleterious effect on productivity and/or worth of an animal. This can be used to infer or predict the phenotype of an animal, for example, identify whether or not an animal is suitable for (or at least more or less suitable for) production and/or breeding purposes, identify whether a cell or embryo is suitable for use (or at least more or less suitable) in producing an animal which is suitable for production and/or breeding purposes, and to calculate the worth of an animal.

The inventors also contemplate that the analysis of one or more genetic marker which is in linkage disequilibrium with the specific marker they have identified may also be used for the same purpose. In addition, haplotypes including the genetic marker of the invention and/or one or more marker in linkage disequilibrium therewith may also be used for this purpose.

The specific genetic variation identified by the inventors' results in a missense variant. Accordingly, methods of the invention may comprise the analysis of the amino acid sequence of PLCD4, fragments, isoforms, and/or precursors thereof, and/or of the levels (including amount, expression and activity levels) of PLCD4. The invention may also comprise the analysis of the nucleotide sequence of a PLCD4 transcript or a fragment thereof.

The inventors believe that this is the first time that alterations in PLCD4 have been associated with a deleterious effect on productivity in the animal, and consequently the worth of the animal and thus the first time a link between PLCD4 and productivity has been made. Accordingly, the inventors contemplate that any genetic alteration which disrupts this gene may be deleterious to the productivity of an animal which is homozygous for it. As such, the invention should be taken to encompass methods involving the analysis of nucleic acid from an animal to determine whether or not it includes any such alteration or variation in the gene (including reference to the transcript), analysis of the amino acid sequence of PLCD4 (including reference to fragments, isoforms, and/or precursors thereof) to determine whether or not it includes any alteration that disrupts PLCD4, and/or the level (including the amount and level of expression, for example) and/or activity of PLCD4 to identify whether or not it has a marker linked to a deleterious effect on productivity and/or whether or not an animal is suitable for (or at least more or less suitable for) production purposes, breeding purposes, and/or inclusion in a herd, or whether or not a cell or embryo is suitable for (or at least more or less suitable for) producing an animal that is suitable for production purposes, breeding purposes and/or inclusion in a herd, for example. While the description which follows may focus on the analysis of the nucleotide sequence or amino acid sequence at a particular position, it should be understood to extend to the analysis of the sequence at any other position within the gene, transcript or PLCD4 protein.

The specific genetic variation identified by the inventors is annotated as being in a “splice region” (ie at or near to a splice site). The inventors contemplate that methods of the invention may comprise the analysis of nucleic acids/proteins to identify splice variants which disrupt PLCD4.

Analysis of a biological marker of PLCD4 in accordance with the invention may also be used for selecting or rejecting a cell or embryo. Such analysis may aid in methods for breeding or cloning animals, for example.

Analysis of a biological marker in accordance with the invention may assist in: predicting phenotypic performance, including use in production management systems known as Marker Assisted Selection; the selection or rejection of animals for breeding and/or other purposes; managing animals in order to maximise their individual potential performance and value; estimating the worth or economic value of an animal; improving profits related to selling animals and/or products produced from the animals; improving the genetics of a population of animals by selecting and breeding desirable animals; generating and maintaining herds of animals; cloning animals likely to have or not have a specific trait; predicting the suitability of an animal and/or its offspring to use in different industries and/or breeding programmes or cloning; avoiding carrier×carrier matings; removing an adverse genetic variant from a population; gene editing to remove deleterious genetic variations from cells or embryos used to generate animals; to increase or optimise productivity of an animal or a herd; for example. It should be appreciated that animals may be tested or screened any time during their life, including early at birth or as embryos or foetuses, to predict life time performance and segregated or managed to suit their genotype and therefore predicted phenotype. Any time during their life includes, but is not limited to, early at birth, as gametes, zygotes, embryos, foetuses.

### Definitions

Various method of the invention can be used to identify whether an animal, cell or embryo carries a biological marker linked to a “deleterious effect on productivity and/or worth”.

“Productivity” refers to the level and/or efficiency of production of a particular product by an animal.

In one embodiment, it includes efficiency of production per unit liveweight of an animal. For example, the product may be meat or milk. Such raw products may be further processed into consumer products such as milk powder, cheeses, whey protein, specialty beverages and meat products. Productivity can be measured based on an assessment of one or more parameters, having regard to the product to be produced, as will be understood by persons skilled in the art. However, by way of example, where the product is milk (or a derivative thereof), milk yield, protein yield, and/or fat yield can be assessed, alone or in combination with one or more other animal assessment parameters as described herein. By way of further example, where the product is meat (or a derivative thereof), liveweight can be assessed, alone or in combination with one or more other animal assessment parameters as described herein.

As used herein the “worth” of an animal refers to an index used to evaluate the value of an animal, for breeding purposes, production purposes, inclusion in a herd, herd management, for example. The “worth” is the sum of the estimated value of one or more characteristics (or animal assessment parameters) which may be associated with the animal, typically weighted by an economic value.

Exemplary assessment parameters may include those which may be referred to as “production traits” and those which may be referred to as “traits other than production” or “TOP”; as described in “Evaluation System for Traits Other Than Production (TOP) for Dairy Cattle in New Zealand” published in August 2014 by Advisory Committee on Traits Other than Production at dairynz.co.nz/media/1205535/TOP Booklet A4.pdf and “Your Index Your Animal Evaluation System (Second Edition)” published by NZ Animal Evaluation Unit at dairynz.co.nz/media/581307/Your_Index_Your_AE_System.pdf. However, persons skilled in the art to which the invention relates will readily appreciate alternative or additional assessment parameters which may be included in any calculation to estimate an animal's worth.

The term “worth” should be taken to encompass “breeding worth”, “lactation worth”, “production worth” and other known indexes used to assess the value of an animal. Breeding worth, lactation worth and production worth, are described in “Your Index Your Animal Evaluation System (Second Edition)” as detailed above, for example. Other exemplary indexes include the Balanced Performance Index (BPI) in Australia, and the Economic Breeding Index (EBI) applied in Ireland. These are described, for example, in “Your Herd. Your Test. Your Future. National Breeding Objective—Final Report” published by Dairy Australia and the Australian Dairy Herd Improvement Scheme at adhis.com.au/v2/downv2.nsf/(ContentByKey)/d356f55a05cac8fDea257c78000a2677/file/nbo %20fin al %20short %20report.pdf?open and “Understanding the Economic Breeding Index (EBI)” published by the Agricultural and Food Development Authority in Ireland at teagasc.ie/media/website/animals/dairy/Understanding_EBI_PTA_BV_Spring_2014.pdf.

Persons skilled in the art to which the invention relates will readily appreciate methods and formulae suitable for estimating worth on the basis of any number of different characteristics. However, by way of example, the methods described in the documents listed in this and the preceding paragraph could be used. Results, data and/or information generated by one or more method of the invention may be used in calculations for estimating “worth”. In embodiments of the invention, an animal may be selected or rejected for a particular purpose (for example, inclusion in a herd, for production purposes and/or for breeding purposes) based on its worth.

In certain embodiments, the methods of the invention are described in terms of estimating the worth of an animal “and/or its offspring”. This should be taken to mean that the worth of the offspring of an animal or pair of animals can be estimated indirectly based on evaluation of one or both parents without the need to directly assess the offspring. Such evaluation would at least involve identifying whether or not one or both parent has a biological marker in accordance with the invention. For example, the method may comprise identifying whether or not a parent animal has a biological marker linked to a deleterious effect on productivity and/or worth of an animal and using this information to in a calculation to estimate the worth of the offspring. Such methods may be used to estimate the potential worth of an offspring if a pair of chosen animals was mated, for example. Alternatively, it could be used to estimate the worth of an existing offspring. Such methods may later be followed up with direct analysis or observation of the offspring, if desired. Persons skilled in the art will readily appreciate methods for estimating the worth of an offspring according to this embodiment of the invention having regarding to the description provided herein and known methods for estimating worth, as hereinbefore described, for example.

In one embodiment of the invention, worth is calculated taking into consideration one or more of the animal assessment parameters (or traits) of milk yield, milk fat yield and milk protein yield (production traits), liveweight, stature, body condition score, rump width, milking speed, capacity, dairy conformation, overall opinion and adaptability (TOP), alone or in combination with one or more other animal assessment parameters. In one embodiment, worth is calculated taking into consideration one or more of protein yield, milk yield, and fat yield, alone or in combination with one or more other animal assessment parameters. In one embodiment, worth is calculated taking into consideration one or more of protein yield, milk yield, fat yield, and liveweight, alone or in combination with one or more other animal assessment parameters. In one embodiment, worth is calculated taking into consideration one or more of liveweight, stature, body condition score, rump width, milking speed, capacity, dairy conformation, overall opinion and adaptability, alone or in combination with one or more other animal assessment parameters. In one embodiment, worth is calculated taking into consideration one or more of stature, body condition score, rump width, milking speed, capacity, dairy conformation, overall opinion and adaptability, alone or in combination with one or more other animal assessment parameters. In one embodiment of the invention, worth is calculated taking into consideration all of the production traits of milk yield, milk fat yield and milk protein yield, alone or in combination with one or more other animal assessment parameters. In one embodiment, worth is calculated taking into consideration all of protein yield, milk yield, fat yield, and liveweight, alone or in combination with one or more other animal assessment parameters. In another embodiment, worth is calculated taking into consideration all of the TOP of liveweight, stature, body condition score, rump width, milking speed, capacity, dairy conformation, overall opinion and adaptability, alone or in combination with one or more other animal assessment parameters. In one embodiment, worth is calculated taking into consideration all of stature, body condition score, rump width, milking speed, capacity, dairy conformation, overall opinion and adaptability, alone or in combination with one or more other animal assessment parameters. In another embodiment of the invention, worth is calculated taking into consideration all of milk yield, milk fat yield and milk protein yield, liveweight, stature, body condition score, rump width, milking speed, capacity, dairy conformation, overall opinion and adaptability, alone or in combination with one or more other animal assessment parameters.

A “deleterious effect on productivity” should be understood to be mean any level of detrimental effect on the level and/or efficiency of production of one or more product by an animal. In one embodiment, it means the animal has or is predicted to have a reduced level of production compared to an animal or animals having at least a desirable level of productivity or the same animal if it did not have a genetic variation which disrupts the PLCD4 gene. In some embodiments, there is at least a 5% level of reduction, at least a 10% level of reduction, at least a 15% level of reduction, at least a 20% level of reduction, at least a 25% level of reduction, at least a 30% level of reduction, at least a 35% level of reduction or at least a 40% level of reduction in the production of one or more product by the animal compared to an animal or animals having at least a desirable level of productivity or the same animal if it did not have a genetic variation which disrupts the PLCD4 gene, for example. In one embodiment, the product is milk and the animal has or is predicted to have a reduced level of milk yield, protein yield and/or fat yield. In another embodiment, the product is meat and the animal has or is predicted to have a reduced liveweight. In another embodiment there is at least a 5% level of reduction, at least a 10/level of reduction, at least a 15% level of reduction, at least a 20% level of reduction, at least a 25% level of reduction, at least a 30% level of reduction, at least a 35% level of reduction or at least a 40% level of reduction in liveweight of the non-human animal. In another embodiment there is at least a 25% level of reduction in liveweight of the non-human animal.

What is considered a “desirable level of productivity” may differ from one animal or breed or farm or herd or country to another, for example. It may also differ depending on the product which an animal is being used to produce. Skilled persons will readily appreciate a benchmark desirable level of productivity having regard to the product to be produced, information on breed, the herd or population of animals used and one or more assessment criteria known in the art. In one embodiment, a desirable level of productivity is the mean of a population of animals. In another embodiment, a desirable level of productivity is the mean of a population of animals that is homozygous for the wild-type allele of PLCD4. In another embodiment, a desirable level of productivity is the mean of a population of animals that is heterozygous for a genetic marker of the invention. In another embodiment, a desirable level of productivity is the level of a chosen highly productive animal.

A “deleterious effect on worth” or like phrases is intended to mean any level of detrimental effect on the estimated worth of an animal. In one embodiment, it means the animal has or is predicted to have a lower worth compared to an animal or animals having a desirable worth or the worth of same animal if it did not have a genetic variation which disrupts the PLCD4 gene. In some embodiments, there is at least a 5% level of reduction, at least a 10% level of reduction, at least a 15% level of reduction, or at least a 20% level of reduction in the worth of an animal compared to an animal or animals having a desirable worth or the worth of same animal if it did not have a genetic variation which disrupts the PLCD4 gene, for example.

What is considered a “desirable worth” may differ from one animal or breed or farm or herd or country to another, for example. It may also differ depending on the purpose for which an animal is to be used, the industry in which an animal is to be used, and/or the product which an animal is being used to produce. Persons skilled in the art will readily be able to appreciate or calculate a benchmark desirable worth having regard to such factors and using one or more known methods to calculate worth of an animal (for example, those herein before described). In one embodiment, a desirable worth is the mean of a population of animals. In another embodiment, a desirable worth is the mean of a population of animals that is homozygous for the wild-type allele of PLCD4. In another embodiment, a desirable worth is the mean of a population of animals that is heterozygous for a genetic marker of the invention. In another embodiment, a desirable worth is the worth of a chosen animal.

In one embodiment, “a deleterious effect on productivity and/or worth” includes reference to a deleterious effect on one or more animal assessment parameters (or traits) representative of productivity and used in worth estimations. These traits include “production traits” and “traits other than production (TOP)”, as described herein before. In one embodiment, the one or more animal assessment parameters representative of productivity include the “production traits” protein yield, milk yield, fat yield, and the “TOP” liveweight, stature, body condition score, rump width, milking speed, capacity, dairy conformation, overall opinion and adaptability. Accordingly, in one embodiment the invention provides methods to identify whether or not an animal, cell or embryo carries a biological marker linked to a deleterious effect on one or more of protein yield, milk yield, fat yield, liveweight, stature, body condition score, rump width, milking speed, capacity, dairy conformation, overall opinion and adaptability, and the various aspects of the invention described herein should be taken to include this. In one embodiment, the invention provides methods to identify whether or not an animal, cell or embryo carries a biological marker linked to a deleterious effect on one or more of protein yield, milk yield, and fat yield. In one embodiment, the invention provides methods to identify whether or not an animal, cell or embryo carries a biological marker linked to a deleterious effect on one or more of protein yield, milk yield, fat yield, and liveweight. In one embodiment, the invention provides methods to identify whether or not an animal, cell or embryo carries a biological marker linked to a deleterious effect on one or more of liveweight, stature, body condition score, rump width, milking speed, capacity, dairy conformation, overall opinion and adaptability. In one embodiment, the invention provides methods to identify whether or not an animal, cell or embryo carries a biological marker linked to a deleterious effect on one or more of stature, body condition score, rump width, milking speed, capacity, dairy conformation, overall opinion and adaptability. In another embodiment, the invention provides methods to identify whether or not an animal, cell or embryo carries a biological marker linked to a deleterious effect on all of the following: protein yield, milk yield, and fat yield. In another embodiment, the invention provides methods to identify whether or not an animal, cell or embryo carries a biological marker linked to a deleterious effect on all of the following: liveweight; protein yield; milk yield; and fat yield. In another embodiment, the invention provides methods to identify whether or not an animal, cell or embryo carries a biological marker linked to a deleterious effect on all of the following: stature; body condition score; rump width; milking speed; capacity; dairy conformation; overall opinion; and adaptability. In another embodiment, the invention provides methods to identify whether or not an animal, cell or embryo carries a biological marker linked to a deleterious effect on all of the following: liveweight, stature, body condition score, rump width, milking speed, capacity, dairy conformation, overall opinion and adaptability. In one embodiment, the invention provides methods to identify whether or not an animal, cell or embryo carries a biological marker linked to a deleterious effect in all of the following: protein yield, milk yield, fat yield, liveweight, stature, body condition score, rump width, milking speed, capacity, dairy conformation, overall opinion and adaptability.

Persons of general skill in the art to which the invention relates will readily understand the meaning and nature of the animal assessment parameters of “Protein yield”, “Milk yield”, “Fat yield”, “Liveweight”, “Stature”, “Body condition score”, “Rump width”, “Milking speed”, “Capacity”, “Dairy conformation”, “Overall opinion”, and “Adaptability”. However, they are described, for example, in “Evaluation System for Traits Other Than Production (TOP) for Dairy Cattle in New Zealand” and “Your Index Your Animal Evaluation System (Second Edition)” as referred to herein before. When assessing an animal, the animal assessment parameters may be assigned a measure or a score. In one embodiment, when assessing an animal stature, body condition, rump width, capacity, dairy conformation, overall opinion, milking speed, liveweight and/or adaptability may be assigned a score on a scale. Persons of skill in the art will readily appreciate appropriate scales of use. However, by way of example, the scales described in “Evaluation System for Traits Other Than Production (TOP) for Dairy Cattle in New Zealand” could be used. In certain embodiments, liveweight may be assigned a measure in Kilograms (or other appropriate units of measure), stature may be assigned a measure in centimeters (or other appropriate units of measure), and protein yield, milk yield, and/or fat yield may be assigned a measure in Kg/day (or other appropriate units of measure), for example.

A “deleterious effect on one or more animal assessment parameters” or like phrases is intended to mean any level of detrimental effect on an assessment parameter. In the context of the invention, for stature, body condition, rump width, capacity, dairy conformation, overall opinion, adaptability, liveweight, protein yield, milk yield, and/or fat yield a low measure or score is considered undesirable. In the context of the invention, for milking speed a high measure (eg a fast speed) or score is considered undesirable. In one embodiment, a “deleterious effect on one or more animal assessment parameters” means the animal has or is predicted to have a lower measure or lower score for stature, body condition, rump width, capacity, dairy conformation, overall opinion, liveweight, protein yield, milk yield, and/or fat yield compared to an animal or animals having a desirable assessment measure or score or the same animal if it did not have a genetic variation which disrupts the PLCD4 gene. In one embodiment, a “deleterious effect on one or more animal assessment parameters” means the animal has or is predicted to have a higher measure or score for milking speed (ie. an increased milking speed, which in the context of the invention is likely to be indicative of a lower milk volume) compared to an animal or animals having a desirable assessment measure or score or the same animal if it did not have a genetic variation which disrupts the PLCD4 gene. In certain embodiments, there is at least a 5% level of difference, at least a 10% level of difference, at least a 15% level of difference, or at least a 20% level of difference in the assessment measure or score for the animal compared to an animal or animals having a desirable assessment measure or score or the same animal if it did not have a genetic variation which disrupts the PLCD4 gene, for example.

What is considered a “desirable assessment measure” or “desirable assessment score” may differ from one animal or breed or farm or herd or country to another, for example. It may also differ depending on the product which an animal is being used to produce. Persons skilled in the art will readily be able to appreciate or calculate a benchmark desirable assessment measure or score using one or more known methods (for example, those described in “Evaluation System for Traits Other Than Production (TOP) for Dairy Cattle in New Zealand” and “Your Index Your Animal Evaluation System (Second Edition)” as referred to herein before). In one embodiment, a desirable assessment measure or score is the mean value in a population. In another embodiment, a desirable assessment measure or score is the mean of a population of animals that is homozygous for the wild-type allele of PLCD4. In another embodiment, a desirable assessment measure or score is the mean of a population of animals that is heterozygous for a genetic marker of the invention. In another embodiment, a desirable assessment measure or score is the value of a specific individual.

The term “biological marker(s)” as used herein should be taken broadly and includes, for example, genetic markers, the level of a protein (including reference to a fragment thereof, a precursor thereof, an isoform thereof) or a nucleic acid encoding a protein (including reference to a fragment thereof, a precursor thereof, an isoform thereof), the level of expression of a gene or protein (including reference to a fragment thereof, a precursor thereof, an isoform thereof), the level of activity of a protein (including reference to a fragment thereof, a precursor thereof, an isoform thereof) and/or variation in the amino acid sequence of a protein (including reference to a fragment thereof, a precursor thereof, an isoform thereof) which may include observation of the size of a protein (including reference to a fragment thereof, a precursor thereof, an isoform thereof) or nucleic acid (including for example a transcript). In one embodiment, a biological marker comprises a splice variant.

The term “genetic marker” as used herein refers to nucleic acids or specific genetic loci (including specific nucleotide positions) that are polymorphic or contain sequence alterations or variations within a population, the alleles of which can be detected and distinguished by one or more analytic methods.

The term “genetic marker” further includes within its scope a plurality of genetic markers co-segregating, in the form of a “haplotype”. In this context, the term “haplotype” refers to a plurality of genetic markers that are generally inherited together. Typically, genetic markers within a haplotype are in linkage disequilibrium.

Reference herein to “analysing” a nucleic acid or nucleic acid sequence of an animal, cell or embryo to identify whether or not it includes a genetic variation which disrupts the PLCD4 gene and/or a genetic marker in linkage disequilibrium therewith should be taken to include the physical, biological and/or chemical analysis of a nucleic acid from an animal, cell or embryo, as well as the analysis of data already available for the nucleic acid of a particular animal, cell or embryo. References to “analysing” a nucleic acid should be taken to include analysis of DNA or RNA (for example, a PLCD4 transcript) and either or both strands of a double-stranded nucleic acid. Further, reference to nucleotide or nucleic acid “sequence” should be taken broadly to include both a single nucleotide as well as two or more nucleotides. In addition, with reference to nucleic acids which may have a double-stranded state, reference to determining or identifying whether or not a nucleic acid includes a specific genetic marker (such as the 107313998A allele) should be read to encompass determining or identifying the genetic marker by observing the nucleotide or sequence on either strand of a DNA molecule.

Reference to “analysing” an amino acid sequence or a PLCD4, a precursor thereof, a fragment thereof and/or an isoform thereof of an animal, cell or embryo to identify whether or not it includes a variation which disrupts PLCD4 should be taken to include the physical, biological and/or chemical analysis of a PLCD4 protein, a precursor thereof, an isoform thereof, and/or a fragment thereof from an animal, cell or embryo, as well as the analysis of data already available for a PLCD4, a precursor, an isoform, a fragment of a particular animal, cell or embryo.

Reference to “analysing a nucleic acid of a cell or embryo” or “analysing a PLCD4, a fragment thereof, an isoform thereof, and/or a precursor thereof of a cell or embryo” and like phrases should be taken to include reference to analysing a nucleic acid or a PLCD4, a fragment thereof, an isoform thereof, and/or a precursor thereof of an animal from which the cell or embryo is derived in lieu of directly analysing a nucleic acid or a PLCD4, a fragment thereof, an isoform thereof, and/or a precursor thereof of said cell or embryo.

In one embodiment of the invention, the step of “analysing” a nucleic acid of an animal, cell or embryo to identify whether or not it includes a genetic variation which disrupts the PLCD4 gene and/or a genetic marker in linkage disequilibrium therewith may comprise imputing a genotype of said animal (including the genetic variation which disrupts the PLCD4 gene and/or a genetic marker in linkage disequilibrium therewith). In one embodiment, the step of analysing may comprise directly genotyping (for example, involving physical, biological, or chemical analysis of a nucleic acid) the animal, cell or embryo for a marker and comparing the genotype of the animal, cell or embryo to the genotype of a family member for which an additional marker has been genotyped. In one embodiment, the step may comprise identifying a genotype of the animal, cell or embryo for a marker (for example, involving analysis of genetic data already available for a particular animal, cell or embryo) and comparing the genotype of the animal, cell or embryo to the genotype of a family member for which an additional marker has been genotyped. A genetic marker of the invention and/or a marker in linkage disequilibrium is imputed to be present or absent from the animal, cell or embryo based on the comparison.

In another embodiment, the step of “analysing” may comprise directly genotyping the animal, cell or embryo for a marker and comparing the genotype of the animal, cell or embryo to the genotype(s) of a reference population comprising apparently unrelated individuals for which an additional marker has been genotyped. In another embodiment, the step may comprise identifying a genotype of the animal, cell or embryo for a marker (for example, involving analysis of genetic data already available for a particular animal, cell or embryo) and comparing the genotype of the animal, cell or embryo to the genotype(s) of a reference population comprising apparently unrelated individuals for which an additional marker has been genotyped. A genetic marker of the invention and/or a marker in linkage disequilibrium is imputed to be present or absent from the animal, cell or embryo based on the comparison. In one embodiment, a reference population may be assembled from animals of a similar breed or crossbreed composition, or animals otherwise expected to share a similar haplotype structure, for example. Skilled persons will readily appreciate other appropriate reference populations.

Imputation methods are described, for example, in Li, Y., Willer, C., Sanna, S., & Abecasis, G. (2009). Genotype imputation. Annual review of genomics and human genetics, 10, 387. Computational, statistical and/or in silico methods for genotype imputation will be readily known to those skilled in the art to which the invention relates. In some embodiments, these methods may be practised using readily available algorithms, software packages and tools such as, by way of non-limiting example, IMPUTE, MACH, fastPHASE, BIMBAM, PLINK, TUNA, WHAP, FImpute and/or BEAGLE.

It should be appreciated that identifying whether or not an animal, cell or embryo carries a genetic marker linked to a deleterious effect on productivity and/or worth of an animal and/or a genetic marked in linkage disequilibrium, and/or whether or not a nucleic acid has a genetic variation which disrupts the PLCD4 gene and/or a genetic marker in linkage disequilibrium can include identifying by imputation. Similarly, reference to an animal, cell or embryo having been identified to carry or not carry a genetic marker should also be taken to include it having been identified by imputation.

Similar references to “identify”, “identifying” and the like should be construed in the same way, unless the context requires otherwise.

Reference to “observing the level and/or activity of a PLCD4, a precursor thereof, an isoform thereof, a fragment thereof, and/or a nucleic acid encoding any one or more thereof” of an animal, cell or embryo should be taken to include the physical or chemical analysis of the level and/or activity of a PLCD4, a precursor thereof, an isoform thereof a fragment thereof, and/or a nucleic acid encoding any one or more thereof from an animal, cell or embryo, as well as the analysis of data already available for the level and/or activity of a PLCD4, a precursor thereof, an isoform thereof, a fragment thereof, and/or a nucleic acid encoding any one or more thereof of a particular animal, cell or embryo.

Reference to “observing the level and/or activity of a PLCD4, a precursor thereof, an isoform thereof, a fragment thereof, and/or a nucleic acid encoding any one or more thereof” of a cell or embryo should be taken to include reference to observing the level and/or activity of a PLCD4, a precursor thereof, an isoform thereof a fragment thereof, and/or a nucleic acid encoding any one or more thereof of an animal from which the cell or embryo is derived in lieu of directly observing the level and/or activity of a PLCD4, a precursor thereof, an isoform thereof, a fragment thereof, and/or a nucleic acid encoding any one or more thereof of said cell or embryo.

The term “single nucleotide polymorphism” (SNP) refers to nucleic acid sequence variations that occur when a single nucleotide in the genome sequence is altered. A single nucleotide polymorphism may also be a single nucleotide insertion or deletion. The different nucleotides within a SNP are referred to as an allele.

The term “genotype” as used herein means the genetic constitution or nucleotide sequence at one or more genetic locus, in particular the nucleotide sequence of an allele of a genetic locus.

“Linkage disequilibrium” should be taken broadly to refer to the tendency of the presence of an allele at one genetic locus to predict the presence of an allele at one or more other genetic loci (for example a distinct genetic marker). The genetic loci need not necessarily be on the same chromosome. However, in a preferred embodiment, the genetic loci are located on the same chromosome.

One measure of linkage disequilibrium is DELTA², which is calculated using the formula described by Devlin et al (Genomics 29 (2):311-22 (1995)), and is a measure of how well an allele X at a first genetic loci predicts the occurrence of an allele Y at a second genetic loci. A DELTA² value of 1.0 indicates the prediction is perfect (for example, if Y is present then X is present). It should be appreciated that reference to linkage disequilibrium herein should not be taken to imply a DELTA² value of 1.0. In particular embodiments, the linkage disequilibrium between an allele at one genetic locus and an allele at a second genetic locus, has a DELTA² value of at least 0.75, at least 0.80, at least 0.85, at least 0.90, at least 0.95, and most preferably 1.0. Other measures of linkage disequilibrium are described, for example, in the Encyclopedia of Cancer at: link.springer.com/referenceworkentry/10.1007%2F978-3-642-16483-5_3368

Skilled persons will readily appreciate methods for determining whether any two alleles are in linkage disequilibrium. However, by way of example, see Genetic Data Analysis II, Weir, Sinauer Associates, Inc. Publishers, Sunderland, Mass., 1996.

A “variation in the amino acid sequence” of PLCD4, isoform, fragment and/or precursor thereof should be considered broadly to include any change in the amino acid sequence. By way of example only, it should be taken to include substitution of any one or more amino acid, addition of one or more amino acid and/or deletion of one or more amino acid. In one embodiment, a variation may be a truncation of a protein.

A “genetic variation” in the PLCD4 gene should be considered broadly to include any change in the nucleotide sequence. By way of example only, it should be taken to include substitution of any one or more nucleotide, addition of one or more nucleotide and/or deletion of one or more nucleotide. In certain embodiments, a variation may result in frameshift and/or introduction of a premature stop codon.

Where the invention is described in terms of identifying or determining whether or not a nucleic acid includes a genetic variation or a peptide or protein includes a variation, it should be appreciated that such variation is a difference in the nucleic acid or amino acid sequence compared to the nucleic acid sequence or amino acid sequence associated with one or more animal which is known not to carry a biological marker which disrupts PLCD4 and is linked to a deleterious effect on productivity and/or worth. Typically, the variation will be determined in relation to a “reference sequence”. In certain embodiments, the reference sequence is a nucleic acid encoding part or all of a PLCD4 gene (or transcript) and/or a genetic marker in linkage disequilibrium therewith or the amino acid sequence of PLCD4, an isoform, a precursor or a fragment thereof, which is associated with an animal that is known not to carry such a marker. In one embodiment, the nucleic acid reference sequence is SEQ ID No. 1, SEQ ID No. 5 or a part of these. In one embodiment, the amino acid reference sequence is SEQ ID No. 3 or a part thereof. In other embodiments, the reference sequence could be a nucleic acid or peptide or protein having a sequence which is known to be associated with an animal or animals who is/are known to carry a biological marker which disrupts PLCD4 and is linked to a deleterious effect on productivity and/or worth. Accordingly, reference to “variation” should not be restricted to mean that the nucleic acid, peptide or protein being analysed is different from the reference sequence.

Certain embodiments of the invention may be described herein with reference to “a part” of PLCD4, a fragment, precursor or isoform thereof. In one embodiment, such a “part” comprises or consists at least 4, 10, 20, 30, 40, 50, 60, 70, 80 or more consecutive amino acids of a PLCD4 which includes a variation which disrupts PLCD4 (in one embodiment, it comprises a variation (for example the amino acid Thr) at a position corresponding to position 326 of PLCD4). In one embodiment, it comprises or consists at least 4 consecutive amino acids of SEQ ID No. 4, or at least 10, 20, 30, 40, 50, 60, 70, 80 or more consecutive amino acids of SEQ ID No. 4. In one embodiment, such a “part” comprises or consists at least 4 consecutive amino acids of SEQ ID No. 4, or at least 10, 20, 30, 40, 50, 60, 70, 80 or more consecutive amino acids of SEQ ID No. 4 which includes one or more amino acid variation which disrupts PLCD4.

Certain embodiments of the invention may be described herein with reference to “a region” of the PLCD4 gene or transcript. In one embodiment, the region will encompass a part of a PLCD4 gene or transcript in which a genetic alteration which disrupts the PLCD4 gene resides. In one embodiment, such a region comprises or consists at least 4 consecutive nucleotides, or at least 10, 20, 30, 40, 50, 60, 70 80 or more consecutive nucleotides of a PLCD4 gene or transcript. In one particular embodiment, the region will encompass a part of a PLCD4 gene or transcript in which the genetic marker 107313998A and/or a marker in linkage disequilibrium therewith resides. In one embodiment, such a region comprises or consists at least 4 consecutive nucleotides of SEQ ID No. 2 or SEQ ID No. 6, or at least 10, 20, 30, 40, 50, 60, 70 80 or more consecutive nucleotides of SEQ ID No. 2 or 6.

Reference is made herein to “isoforms” and “precursors” of a PLCD4 protein. Persons skilled in the art to which the inventions relate will readily understand these terms. However, for the avoidance of any doubt, protein isoforms are different forms of a protein coded from the same gene. For example, different forms of a protein can be produced by alternative splicing of RNA transcripts to form different mRNA sequences or by different glycosylation or other posttranslational modification. For the avoidance of any doubt, protein precursors (including pre-pro-proteins or pre-pro-peptides and pro-proteins or pro-peptides) are inactive forms of a protein or peptide that can be activated by post- and/or co-translation modification.

Reference is also made herein to “fragments” of PLCD4 (including reference to an isoform or precursor thereof). Skilled persons will readily appreciate the meaning of this term having regard to the nature of the invention and the fact that a variation in the amino acid sequence of PLCD4 could be detected without the need to analyse the whole PLCD4 (including reference to an isoform or precursor thereof). A “fragment” may include peptides of any length suitable to identify an amino acid variation in accordance with the invention. A “fragment” will include or encompass the amino acid variation to be detected (for example, one or more amino acid substitution, addition or deletion at one or more particular position(s) in PLCD4). In the case of a variation being a truncation of the PLCD4 protein, a “fragment” may consist of the truncated protein.

A genetic alteration or variation which “disrupts” the PLCD4 gene, may be any genetic change which has an affect on the level, expression or activity of the PLCD4 gene product (including reference to isoforms, fragments and/or precursors thereof). By way of example, it may decrease the level of expression or alter the structure or function of the gene product. Similarly, a variation or alteration in the amino acid sequence which disrupts PLCD4 may be any change in the amino acid sequence of PLCD4 which has an effect on the level or level of activity of PLCD4 (including reference to isoforms, fragments and/or precursors thereof). The term “disrupts” should not be taken to imply that there is substantially no PLCD4 or activity, although this may be preferred, but rather taken to encompass any change in the level or activity, in one embodiment a decreased level or level of activity. According to the invention, a variation which “disrupts” PLCD4 or its gene is one which is associated with a deleterious effect on productivity and/or worth in an animal, as herein before described.

One can readily determine whether a variation disrupts PLCD4 or the PLCD4 gene using standard assays known in the art. However, by way of example, expression levels can be measured using quantitative reverse transcriptase polymerase chain reaction (qRT-PCR), northern blotting, microarray analysis, RNA sequencing for measuring cDNA levels and western blotting, PAGE, mass spectrometry, and immunoprecipitation for measuring protein levels. In addition, and by way of example only, the methodology described by Kumar et al, 2009 (Predicting the effects of coding non-synonymous variants on protein function using the SIFT algorithm; Nature Protocols, Vol 4, No. 8, pp1073-1082) could be used to assist in identifying which variations may disrupt PLCD4 and/or the PLCD4 gene.

In general, where reference is made to an “increase” or “decrease” in the level or activity of PLCD4, an isoform thereof, a fragment thereof, a precursor thereof, and/or a nucleic acid encoding any one or more thereof it should be taken broadly to include any increase or decrease in said level or activity compared to a reference animal or animals or a standard. In one particular embodiment, the reference animal or animals are those having wild-type PLCD4. In another embodiment, the reference animal or animals are those having a genetic or amino acid variation which is linked to or associated with a deleterious effect on productivity and/or worth (ie a variation which disrupts PLCD4). Reference may also be made herein to a “higher” or a “lower” level or activity of PLCD4, an isoform thereof, a fragment thereof, a precursor thereof, and/or a nucleic acid encoding any one or more thereof compared to a reference animal or animals or a standard. This should not be taken to imply a particular level or activity of PLCD4, an isoform thereof, a fragment thereof, a precursor thereof, and/or a nucleic acid encoding any one or more thereof. One can readily determine whether a variation results in an increase or decrease in the level or activity of PLCD4 a higher or lower level or activity of PLCD4 compared to a standard using standard assays known in the art, including those techniques exemplified herein after.

In one embodiment the inventors contemplate that the level or level of activity of PLCD4 in a cell, embryo or animal having a variation which disrupts PLCD4 and/or the PLCD4 gene will be at least approximately 20%, at least approximately 30/, at least approximately 40% or at least approximately 50% lower than an animal or animals which do not have a genetic variation which disrupts PLCD4/the PLCD4 gene (including reference to the same animal if it it did not have such variation). In one particular embodiment, the inventors contemplate that the level would be significantly lower.

Reference to the “PLCD4 gene” should be taken to include reference to the coding and non-coding regions of the gene, including upstream and downstream regulatory elements.

While the inventors have identified the biological markers of the invention in bovine animals, they contemplate that it is equally applicable to a variety of different mammals. Various mammals are used for product productions purposes and/or may be assessed using a worth calculation. Accordingly, the term “animal” is used herein primarily in reference to mammals. In one particular embodiment, the mammal is a ruminant. In another embodiment, the mammal is one within within the Bovidae family. In particular embodiments, the animal is a bovine animal. More particularly the animal is Bos taurus or Bos indicus. In one particular embodiment the animal is a beef or dairy breed. By way of further example, the animal may be chosen from the group of animals including, but not limited to, Jersey, Holstein-Friesian, Ayrshire, crossbred dairy cattle, Angus, Hereford, Simmental and crossbred beef cattle.

A “functionally equivalent variant” of any particular nucleic acid, protein or peptide referred to herein should be taken broadly to encompass any nucleic acid, peptide or protein whose sequence may vary from the specific sequence provided but which nucleic acid, peptide or protein retains substantially the same function; for example, in the case of an oligonucleotide used to detect a genetic marker of the invention, the ability to bind to a particular target nucleic acid or prime a particular reaction with the desired specificity). The phrase “functionally equivalent” should not be taken to imply that the variant has the same level of activity as the nucleic acid, peptide or protein of which it is a variant, although this may be desired. In one embodiment, “functionally equivalent variants” of any particular nucleic acid, peptide or protein will have at least approximately 80%, approximately 90%, approximately 95/, or approximately 99% sequence homology or similarity to the sequence of which they are a variant. In one particular embodiment, the “functionally equivalent variants” of any particular nucleic acid, peptide or protein will have at least approximately 80%, approximately 90%, approximately 95%, or approximately 99% sequence identity to the sequence of which they are a variant.

It should be appreciated that where methods of the invention relate to breeding an animal (or animals), any appropriate breeding methods may be utilised including for example natural insemination, artificial insemination and in vitro fertilisation. Accordingly, the word “mating” should be construed broadly and not limited to the physical pairing of two animals.

As noted previously herein, the methods of the invention may be used to identify animals suitable for cloning. They may also be used during cloning processes, to determine for example whether or not a cell, embryo or cloned animal has or doesn't have a genetic variation in the PLCD4 gene. Any appropriate cloning method could be used. However, by way of example, such cloning techniques include somatic cell nuclear transfer, chromatin transfer, and embryo splitting. Persons of general skill in the art will readily appreciate appropriate somatic cell nuclear transfer and chromatin transfer methodologies. However, by way of example, the methods described in the following publications may be used: Bovine somatic cell nuclear transfer, Ross P J and Cibelli, J B 2010. Methods in Molecular Biology 636: 155-177; and, Influence of cloning by chromatin transfer on placental gene expression at Day 45 of pregnancy in cattle. Mesquita F S, Machado S A, Dmevich J, Borowicz P, Wang Z, Nowak R A. Anim Reprod Sci. 2013 Jan. 30; 136(4):231-44. doi: 10.1016/j.anireprosci.2012.10.030. Epub 2012 Nov. 8.

Where IVF is employed in the context of the invention, any appropriate IVF methodology may be used, as will be apparent to persons of general skill in the art to which the invention relates. However, by way of example, appropriate methods are described, for example, in: Imai K, Tagawa M, Yoshioka H, Matoba S, Narita M, et al. (2006) The efficiency of embryo production by ovum pick-up and in vitro fertilization in cattle. J Reprod Dev 52: 19-29.

In certain embodiments, methods of the invention may involve taking a sample from an animal to be tested. The sample may be any appropriate tissue or body fluid sample. In one embodiment, the sample is one or more of a cell, blood, muscle, bone, somatic cell(s), saliva, or semen. Such samples can be taken from the animal using standard techniques known in the art. It should be appreciated that a sample may be taken from an animal at any stage of life, including prior to birth; by way of non-limiting example, a zygote, an embryo, a feotus. Individual gametes could also be tested using the methods of the invention. This may assist in breeding and/or cloning programmes. Accordingly, “sample” should be taken to include a zygote, embryonic tissue, foetal tissue and gametes. A sample may also be taken after the death of an animal. The samples are analysed using techniques which allow for the observation or analysis of a biological marker, as will be described further herein after.

In addition, it should be appreciated that where analysis or observation of a biolocial marker in an animal is conducted during gestation, the analysis or observation could be conducted by analysing protein, peptide, nucleic acid or a cell of that animal that may be present in the maternal blood supply, placenta, amniotic fluid or any other maternal tissue or fluid prior to birth of the animal. Accordingly, reference to analysing a nucleic acid of an animal, analysing PLCD4, a precursor, an isoform and/or fragment thereof of an animal, observing the level or activity of a PLCD4, a precursor, an isoform and/or fragment thereof of an animal, and the like, should be taken to include reference to analysing and/or observing one or more of these from that animal that may be present in a maternal tissue or fluid.

“Embryo” should be taken broadly to include an organism from the first division of the zygote. In certain embodiments, an embryo is an organism between the first division of the zygote until the time it becomes a foetus. Reference to an “embryo” should be taken to include reference to an organism at different developmental stages, including a blastula, blastocyst, gastrula, and morula for example.

In one aspect, the invention provides methods for the selection or rejection of one or more cells. In certain embodiments, such “cells” may include a gamete (for example, sperm or ovum) or zygote. Selection of such cells may be of use in an IVF program, for example. In other embodiments, such “cells” may be somatic cells, embryonic cells, embryonic stem cells, cells in a cell line, cells of use in cloning, for example. Selection of these cells may be of use in cloning procedures, or preparing cell lines for use in cloning and other procedures, for example.

Certain aspects and embodiments of the invention may be described herein with reference to “fusing a first and a second gamete” to form a zygote. This and like phrases should be taken broadly to include fertilisation processes, such as may be used in in vitro fertilisation processes. Skilled persons will readily appreciate standard means of “fusing” gametes to form a zygote.

For ease of reference, the methods of the invention may be described herein after in terms of analysing a biological marker (such as a nucleic acid sequence, amino acid sequence, level of a protein and peptide or activity level of a protein or peptide) in, of or from an “animal” or to determine whether or not an “animal” has a particular marker linked to a deleterious effect on productivity and/or worth or determining the genotype of an “animal”, and the like. It should be appreciated that the methods of the invention are also applicable to analysing and determining whether or not individual cells, including gametes, and embryos may have relevant biological markers. Accordingly, reference to “animals” should also be taken to include reference to cells or embryos, unless the context requires otherwise.

The invention may be described herein with reference to a level or level of activity of PLCD4 (including reference to an isoform, precursor or fragment thereof and/or a nucleic acid encoding any one or more thereof) being “indicative” of an animal or animals which is/are known not to carry a biological marker linked to a deleterious effect on productivity and/or worth of an animal, or being “indicative” of an animal or animals which is/are known to carry a biological marker linked to a deleterious effect on productivity and/or worth of an animal. “Indicative” should be taken to mean a level which is associated with an animal or animals known to have or not to have a variation in an amino acid or nucleic acid sequence which disrupts PLCD4/the PLCD4 gene. It should not be taken to mean that the level is exactly the same as a level associated with such animal or animals. However, in one embodiment the level or level of activity is substantially similar or substantially the same as that associated with such animal or animals. Reference to “animal or animals” should be taken to mean that a particular reference or standard is based on a value from a single animal or pooled or averaged from a group of animals.

Reference may be made herein to comparisons between a particular parameter (such as a level of PLCD4, or the worth of an animal, or the suitability of animal for a particular purpose) for an animal (or cell or embryo) and a benchmark for the same parameter of an “animal” or “animals” (or cell or cells or embryo or embryos) having specified characteristics: for example, in one embodiment, it may be stated that an animal is more or less suitable for a particular purpose than an “animal or animals” which do not include a biological marker linked to a deleterious effect on productivity and/or worth of an animal. Reference to an “animal” or “animals” should be taken to mean that the relevant benchmark is based on a value from a single animal or pooled or averaged from a group of animals. The same is the case for a cell or cells or embryo or embryos.

The various embodiments of the invention may be described herein with reference to a reference animal (or animals) being one which is known to carry a biological marker linked to a deleterious effect on productivity and/or worth of an animal or being one which is known not to carry a biological marker linked to a deleterious effect on productivity and/or worth of an animal. Similarly, the various embodiments of the invention may be described herein which reference to a standard or control being one which is associated with an animal (or animals) known to carry a biological marker linked to a deleterious effect on productivity and/or worth of an animal or being one which is associated with an animal (or animals) known not to carry a biological marker linked to a deleterious effect on productivity and/or worth of an animal. In certain embodiments of the invention, a combination of both types of reference animal(s), standard(s) and/or control(s) could be used. In addition, while various embodiments of the invention may be described herein with reference to making a decision (such as a selection decision) based on a parameter being tested being “indicative” of an animal (or animals) known to carry a relevant biological marker, it should be appreciated that in some embodiments decisions could alternatively be made on the basis of the parameter being “indicative” of an animal (or animals) known not to carry a relevant biological marker. In certain embodiments, a combination of both could be used. The description should be read accordingly, unless the context requires otherwise.

Reference is made herein to the methods of the invention being used to identify whether or not an animal, cell or embryo is “suitable” (or at least “more or less suitable”) for production purposes or breeding purposes or to identify whether or not an animal is “suitable” (or at least “more or less suitable”) for inclusion in a herd. In one embodiment, the production purpose is milking. In one embodiment, the production purpose is meat production. In one embodiment, a cell, animal or embryo is identified as unsuitable or less suitable where it includes a biological marker linked to a deleterious effect on productivity and/or worth of an animal. In one embodiment, a cell, animal or embryo is identified as suitable or more suitable where it does not include a biological marker linked to a deleterious effect on productivity and/or worth of an animal. “More or less” suitable refers to a comparison between the animal, cell or embryo and an animal, cell or embryo that has (or in some embodiments does not have) a biological marker linked to a deleterious effect on productivity and/or worth of an animal, or a comparison between the same animal, cell or embryo if it had (or in some embodiments did not have) a biological marker linked to a deleterious effect on productivity and/or worth of an animal.

References herein to singular terms such as “a” and “an” and the like herein should be taken to include reference to “one or more”.

Genetic Marker(s)

In one aspect, a method of the invention involves the analysis of a nucleic acid from an animal to determine whether or not it includes a genetic marker as herein before described (for example, a genetic alteration which disrupts the PLCD4 gene and/or a genetic marker in linkage disequilibrium therewith). It should be appreciated that the invention may employ analysis of the gene encoding PLCD4 and/or analysis of a transcript encoding PLCD4, or a part thereof.

The specific marker identified by the inventors is a substitution of a G with an A at position 107313998 within the PLCD4 gene on chromosome 2 of Bos taurus. This alteration in the gene results in a missense variant of PLCD4 (Ala is replaced with Thr at a position corresponding to amino acid position 326 of PLCD4). The two alleles of this marker may be referred to herein as 107313998G (wild type) and 107313998A (mutant). The Entrez Gene ID for the PLCD4 gene is (http://www.ncbi.nlm.nih.gov/gene/540771). The sequence and position is based on the genomic sequence of chromosome 2 in bovine build UMD3.1.1 (AC_000159.1 in the GenBank database http://www.ncbi.nlm.nih.gov/). Further sequence information is provided in SEQ ID No. 1 (wild-type) and SEQ ID No. 2 (mutant) herein after.

It will be appreciated that the precise location of a particular genetic marker of the invention may vary slightly from genome to genome; for example, in a different species of animal, or different breed of animal, the location of the marker may vary. However, persons of skill in the art to which the invention relate will be able to readily identify a particular marker in different genomes through routine sequence alignment and with knowledge that it resides in the PLCD4 gene. To account for this variation in the location of a genetic marker across genomes reference to a position of a particular marker herein should be taken to mean a position “corresponding to” a particular position of chromosome 2 of Bos taurus” in the UMD3.1.1 genome build. For example, reference to the genetic marker 107313998A on chromosome 2 of Bos Taurus should be taken to mean that the genetic marker is at a position corresponding to position 107313998 on chromosome 2.

Similarly, it will be appreciated that the precise location of a particular biological marker of the invention may vary slightly from one transcript or protein to another, for example, in a different species of animal, or different breed of animal, the location of the marker may vary. However, persons of skill in the art to which the invention relates will be able to readily identify a particular marker in different transcripts or proteins through routine sequence alignment. To account for this variation in the location of a marker across transcripts or proteins reference to a position of a particular marker herein should be taken to mean a position “corresponding to” a particular position of a transcript or protein reference sequence. For example, reference to a marker of the invention being located at a position or in a particular region of a transcript should be taken to mean that the marker on any given transcript is at a position or in a region “corresponding” to the position(s) (ie position or the region may be defined by different positions in a different transcript). Similarly, reference to a marker of the invention being located at a particular position or in a particular region of the protein should be taken to mean that the marker of any given protein is at a position or in a region “corresponding” to those positions (ie the position or region may be defined by different positions in a different protein).

While the inventors have observed that the genetic marker 107313998A is linked to a deleterious effect on productivity and/or worth of an animal, they contemplate that any variation in the nucleotide sequence at this genetic position may be linked to a deleterious effect on productivity and/or worth of an animal. This includes deletion, addition or substitution at this position. The invention should be interpreted accordingly.

In one embodiment, the methods of the invention involve the analysis of a nucleic acid to determine whether or not it includes a variation in the nucleotide sequence (or codon) coding for the amino acid at a position corresponding to position 326 of PLCD4. Such variation may result in substitution, deletion or addition of one or more amino acid at the position corresponding to position 326 of PLCD4.

In one embodiment, the methods involve the analysis of a nucleic acid to determine whether or not it includes the genetic marker 107313998A on chromosome 2 of Bos taurus. Alternatively, or in addition, the methods may involve analysing the nucleotide sequence of a nucleic acid to determine the nucleotide sequence of a genetic marker in linkage disequilibrium with this genetic marker. In one particular embodiment, the methods of the invention may involve analysing the nucleotide sequence of a nucleic acid to determine the haplotype. In one embodiment, the haplotype includes a genetic marker at a position corresponding to position 107313998 on chromosome 2 of Bos taurus.

In one embodiment, where the genetic marker 107313998A and/or a genetic marker in linkage disequilibrium therewith is present it is identified as carrying a biological marker linked to a deleterious effect in productivity and/or worth of an animal and it is inferred that an animal is not suitable for production purposes, breeding purposes or inclusion in a herd, or that a cell or embryo is not suitable for generating an animal for such purposes, for example. In such cases, the animal, cell or embryo may be rejected. In another embodiment, if the animal, cell or embryo has the wild-type allele of the genetic marker or the nucleic acid sequence encoding PLCD4 the animal may be selected for use in a herd or breeding programme or for production purposes, and the cell or embryo may be selected for use in generating an animal for such purposes, for example.

Where a genetic alteration which disrupts the PLCD4 gene involves insertion and/or deletion of one or more nucleotide, it should be appreciated that the methods of the invention may involve analysis of the size of the PLCD4 gene (or transcript) or a part or region thereof.

It should also be appreciated that one could analyse the nucleic acid sequence of either strand of a double-stranded nucleic acid to identify the sequence at a particular genetic locus or position; for example, instead of analysing the strand associated with the sequence variant listed above, the nucleotide sequence of the opposite or complementary strand of DNA could be analysed. Persons of skill in the art will readily appreciate nucleic acid sequence variations on such opposite strand which correlate with the genotypes mentioned above, having regard to the information contained herein and nucleic acid base pairing principles (ie, A pairs with T and C pairs with G). SEQ ID No. 1 and 2 herein after reflect the minus (or reverse) strand relative to the UMD3.1.1 reference. Skilled persons will readily appreciate the sequence of the plus (or forward) strand. Accordingly, while the invention may be described herein in terms of determining whether or not a nucleic includes a specific genetic marker (such as the 107313998A allele) it should be read to encompass determining the genetic marker by observing the nucleotide or sequence on either strand.

As noted above, the invention also encompasses use of a genetic marker which is in linkage disequilibrium with a marker of the invention. Such markers may be analysed instead of or in addition to a genetic marker of the invention.

Nucleic acids can be analysed to determine the genotype/sequence of the genetic markers described herein according to any appropriate technique. Such techniques include for example polymerase chain reaction (PCR), including allele-specific PCR, gel electrophoresis, the use of oligonucleotide probe hybridisation, Southern blotting, direct sequencing, restriction digestion, restriction fragment length polymorphism (RFLP), single-strand confirmation polymorphism (SSCP), LCR (ligase chain reaction), denaturing gradient gel electrophoresis (DGGE), the use of allele-specific oligonucleotides (ASOs), the use of proteins which recognize nucleic acid mismatches, such as E. coli mutS protein, RNAse protection assays, oligonucleotide array hybridisation (for example genotyping microarrays or “SNP chips”), denaturing HPLC (dHPLC), fluorescence quenching PCR (TaqMan™, Applied Biosystems, CA 94404, USA), High Resolution Melting (HRM), matrix-assisted laser desorption/ionisation time-of-flight mass spectroscopy (MALDI-TOF MS), and Genotype-by-Sequencing (GBS) methods that utilise high-throughput sequencing technologies. Combinations of two or more of such techniques may be used. Such combination may increase the sensitivity of the analysis being conducted.

The technique(s) used will depend on the nature of the nucleic acid and/or marker to be detected as will be appreciated by skilled persons. For example, single nucleotide polymorphisms (SNPs), may be analysed using those techniques capable of resolving a single nucleotide difference between sequences; for example, direct sequencing or LCR, allele-specific PCR, RFLP, SSCP, DGGE, using allele-specific oligonucleotides (ASOs), or proteins which recognize nucleic acid mismatches, oligonucleotide array hybridisation, dHPLC, fluorescence quenching PCR and matrix MALDI-TOF MS.

Any one or more of the techniques mentioned hereinbefore (including for example, SSCP, RFLP, DGGE, dHPLC and direct sequencing) may be used to analyse genetic markers which may include insertion or deletion of one or more nucleotide.

It should be appreciated that certain of the techniques of use in analysing a genetic marker in accordance with the invention will utilise one or more oligonucleotides which hybridise to a genetic region encompassing the marker, adjacent to the marker, or flanking the marker. Such oligonucleotides may be DNA, RNA or derivatised forms thereof and include nucleic acid primers, such as PCR and LCR primers, and nucleic acid probes.

Persons of ordinary skill in the art to which the invention relates will readily appreciate appropriate oligonucleotides of use in the invention having regard to one or more of the nucleic acid sequence of chromosome 2, particularly in the genetic regions proximal to the genetic marker, the nature of the genetic marker to be analysed, and the general principles of nucleic acid hybridisation. The nucleic acids will be capable of hybridising in a specific manner to a target nucleic acid and in the case of primers they will be capable of priming a PCR or like reaction. While such nucleic acids will preferably have 100% complementarity to their target region of the mRNA or cDNA of the protein of interest, they may contain one or more non-complementary nucleotides at a particular position while still substantially retaining specificity for the target nucleic acid to which they are designed to bind. By way of example, the nucleic acids may have approximately 80%, approximately 90%₆, approximately 95%, or approximately 99% complementarity or homology to its target. By way of further example, in certain cases, the oligonucleotides may be designed such that a mismatch at a particular nucleotide position is indicative of the nature of the genetic marker being analysed (for example, a SNP). By way of example, a mismatch in the nucleotide present at the 3′ end of an LCR primer will inhibit the reaction providing an indication of the nature of the nucleotide at that position. Mismatches may similarly be utilised in techniques including RNAse protection assays and allele-specific PCR, as well as in fluorescence quenching PCR, for example. Typically, the nucleic acids will hybridise to their target nucleic acid under stringent hybridisation conditions (see for example, Sambrook and Russell, Molecular Cloning: A Laboratory Manual, 2001, Cold Spring Harbor Laboratory Press, Cold Spring Harbor, New York).

The oligonucleotide probes or primers may be of any length as is appropriate for a particular application, having regard to the sequence of the genetic region to which they are designed to bind. A probe or primer will typically be capable of forming a stable hybrid with the complementary sequence to which it is designed to hybridise. Accordingly, the length is dependent on the nucleic acid composition and percent homology between the oligonucleotide and its complementary sequence, as well as the hybridisation conditions which are utilised (for example, temperature and salt concentrations). Such hybridisation factors are well known in the art to which the invention relates. By way of example, oligonucleotides of use in the present invention may be from 2 to 500 nucleotides in length. In one embodiment, particularly where they are used as primers, the oligonucleotides may be of approximately 15 nucleotides to 30 nucleotides in length.

Oligonucleotide probes and primers of use in the invention may be prepared by any number of conventional DNA synthesis methods including recombinant techniques and chemical synthesis, or they may be purchased commercially. It will be appreciated that the usefulness of any probe or primer may be evaluated, at least notionally, using appropriate software and sequence information for the nucleic acid encoding the protein of interest. For example, software packages such as Primer3 (http://primer3.sourceforge.net/), PC Oligo5 (National Bioscience Inc), Amplify (University of Wisconsin), and the PrimerSelect program (DNAStar Inc) may be used to design and evaluate primers.

Where amplification techniques (for example PCR) are used in methods of the invention amplification may be conducted according to conventional procedures in the art to which this invention relates, such as described in U.S. Pat. No. 4,683,202. By way of example PCR reactions will generally include 0.1 μM-1 μM of each primer, 200 μM each dNTP, 3-7 mM MgCl₂, and 1U Taq DNA polymerase.

Further, exemplary PCR cycling conditions include: denaturation at a temperature of approximately 94° C. for 30 to 60 seconds, annealing at a temperature calculated on the basis of the sequence and length of the primer (as herein after discussed) for 30 to 60 seconds, and extension at a temperature of approximately 70° C. to 72° C. for 30 to 60 seconds. By way of example, between 25 and 45 cycles are run.

It will be appreciated by those of ordinary skill in the art that any amplification conditions provided herein are merely exemplary and may be varied so as to optimise conditions where, for example, alternative PCR cyclers or DNA polymerases are used, where the quality of the template DNA differs, or where variations of the primers not specifically exemplified herein are used, without departing from the scope of the present invention. The PCR conditions may be altered or optimised by changing the concentration of the various constituents within the reaction and/or changing the constituents of the reaction, altering the number of amplification cycles, the denaturation, annealing or extension times or temperatures, or the quantity of template DNA, for example. Those of skill in the art will appreciate there are a number of other ways in which PCR conditions may be optimised to overcome variability between reactions.

It will be understood that whilst not specifically exemplified herein, appropriate annealing temperatures for any primer within the scope of the present invention may be derived from the calculated melting temperature of that primer. Such melting temperatures may be calculated using standard formulas, such as that described in Sambrook and Russell, 2001. As will be understood by those of ordinary skill in the art to which this invention relates annealing temperatures may be above or below the melting temperature but generally an annealing temperature of approximately 5C below the calculated melting temperature of the primer is suitable.

Oligonucleotides used for detection and/or analysis of genetic markers in accordance with the invention may be modified to facilitate such detection. Similarly, nucleic acid products obtained using techniques such as PCR may be modified to facilitate detection and/or analysis. For example, the nucleic acid molecules may be labelled to facilitate visual identification using techniques standard in the art. By way of example nucleic acids may be radio-labelled using P³² as may be described in Sambrook and Russell, 2001. Further, nucleic acids may be appropriately labelled for use in colorigenic, fluorogenic or chemiluminescence procedures.

It will be appreciated that the methods of the invention may employ one or more control samples. Such control samples may be positive or negative controls for a particular genetic marker. The type of control samples used may vary depending on such factors as the nature of the genetic marker being analysed and the specific technique being used for such detection and analysis. Positive controls may include samples having known nucleic acid sequences (including nucleic acids of a known size), for example. Negative controls may include samples having no nucleic acid present. By way of general example, positive control samples could include one or more nucleic acids known to have a particular nucleotide sequence at a relevant position. In one embodiment, the method may utilise a control sample having a sequence which is associated with an animal known to carry a genetic marker linked to a deleterious effect on productivity and/or worth of an animal. In another embodiment, the method may utilise a control sample having a sequence which is associated with an animal known not to carry a genetic marker linked to a deleterious effect on productivity and/or worth of an animal.

The methods of this embodiment of the invention may involve comparing the sequence of a nucleic acid being tested to one or more reference sequences, as herein before described.

In one embodiment, the methods of the invention will involve taking a sample from an animal to be tested, as noted herein before.

In order to facilitate detection of a genetic marker in accordance with the invention, a sample may be processed prior to analysis. For example, the sample may be processed to isolate nucleic acid from the sample to be analysed or to amplify a specific genetic region to be analysed.

In one embodiment, nucleic acid is isolated or extracted from the sample prior to analysis. In one embodiment, genomic DNA is isolated or extracted from the sample. In an alternative embodiment, mRNA may be isolated or extracted from the sample. In such a case, the mRNA may be converted to cDNA using reverse transcription techniques known in the art. Techniques for isolating nucleic acids from samples will be readily appreciated by skilled persons. By way of Example, methods of use in isolating nucleic acids are described in Sambrook and Russell, 2001.

In an alternative form of this embodiment of the invention analysis of the nucleic acid may occur in situ obviating the need to extract nucleic acid from the sample. This may be done using PCR for example. Skilled persons will readily appreciate appropriate techniques and methodology to this end (see for example, Sambrook and Russell, 2001).

In one embodiment, the step of analysing a nucleic acid of an animal, cell or embryo to determine whether or not it includes a genetic variation which disrupts the PLCD4 gene and/or a genetic marker in linkage disequilibrium therewith may comprise imputing a genotype of said animal (including the genetic variation which disrupts the PLCD4 gene and/or a genetic marker in linkage disequilibrium therewith), as herein before described. A skilled person will readily appreciate methods for imputing genotypes. However, methods are described, for example, in Li, Y., Willer, C., Sanna, S., & Abecasis, G. (2009). Genotype imputation. Annual review of genomics and human genetics. 10, 387. Computational, statistical and/or in silico methods for genotype imputation will be readily known to those skilled in the art to which the invention relates. In some embodiments, these methods may be practised using readily available algorithms, software packages and tools such as, by way of non-limiting example, IMPUTE, MACH, fastPHASE, BIMBAM, PLINK, TUNA, WHAP, FImpute and/or BEAGLE.

The methods of the invention may be combined with other methods of use in assessing genotype, predicting phenotype, selecting an animal based on certain characteristics, estimating breeding values or estimating worth and the like. Accordingly, the methods of the invention may include, in addition to analysis of a genetic marker identified herein, analysis of additional genetic markers, and/or the level of expression of certain genes/proteins, and/or one or more phenotypic traits, for example.

Peptide and Protein Markers

In another aspect, the methods of the invention comprise analysing a PLCD4, a precursor thereof, an isoform thereof, and/or a fragment thereof to determine whether or not it includes a variation in the amino acid sequence which disrupts PLCD4, in one particular embodiment, one which results in a decrease in the activity of PLCD4. In one embodiment, the variation is located in a region of the protein encoded by the genetic region defined by nucleotides corresponding to positions 107313997 to 107314141 of chromosome 2 of Bos taurus. In one particular embodiment, the variation in the amino acid sequence comprises a substitution of an amino acid. In one embodiment, the variation comprises a variation at a position corresponding to amino acid position 326 of PLCD4. In one embodiment, the variation comprises a substitution of an amino acid at a position corresponding to amino acid position 326 of PLCD4. In one embodiment, the variation comprises a substitution of an Ala amino acid by a Thr amino acid at amino acid position corresponding to amino acid position 326 of PLCD4.

In one embodiment, where the PLCD4, precursor thereof, isoform thereof and/or fragment thereof comprises or consists the amino acid sequence SEQ ID No. 4 or a part thereof an animal, cell or embryo is identified to carry a biological marker linked to a deleterious effect on productivity and/or worth of an animal and it is inferred that an animal is not suitable for production purposes, breeding purposes or inclusion in a herd, or that a cell or embryo is not suitable for generating an animal for such purposes, for example. In such cases, the animal, cell or embryo may be rejected. In another embodiment, if the animal, cell or embryo has the wild-type amino acid sequence the animal may be selected for use in a herd or breeding programme or for production purposes, and the cell or embryo may be selected for use in generating an animal for such purposes, for example.

The amino acid sequences and positions described herein are based on the sequence of PLCD4 in GeneBank NP_001039954.1. Further sequence information is provided in SEQ ID No. 3 (wild-type) and SEQ ID No. 4 (mutant) herein after.

PLCD4, isoforms thereof, precursors thereof, and/or fragments thereof may be analysed using standard techniques known in the art. However, by way of example, peptide sequencing methods, mass spectrometry, Western blotting and ELISA could be used. It should be appreciated that the methods of the invention may involve analysis of the size of PLCD4, a precursor thereof, an isoform thereof, and/or a fragment thereof.

The methods may employ one or more control samples, such as positive and/or negative controls for a particular amino acid sequence variation. The type of control samples used may vary depending on such factors as the type of variation being analysed and the specific technique being used for detection and analysis. Positive controls may include samples having known amino acid sequences (including proteins and peptides of a known size), for example. Negative controls may include samples having no peptide present. In one embodiment, the method may utilise a control sample having a sequence which is associated with an animal known to carry a biological marker linked to a deleterious effect on productivity and/or worth of an animal. In another embodiment, the method may utilise a control sample having a sequence which is associated with an animal known not to carry a biological marker linked to a deleterious effect on productivity and/or worth of an animal.

The methods of this embodiment of the invention may involve comparing the sequence of a peptide or protein being tested to one or more reference sequences, as herein before described.

In one embodiment, the methods of the invention may involve taking a sample for an animal to be tested. In order to facilitate analysis of a peptide in accordance with the invention, a sample may be processed prior to analysis according to any of a number of known methods. For example, the sample may be processed to remove one or more one or more high abundance proteins that might make it difficult to analyse PLCD4, an isoform, fragment or precursor thereof. Exemplary techniques which may be employed to process a sample prior to analysis of PLCD4, an isoform, fragment or precursor thereof are described elsewhere herein.

Level of Peptides and Proteins

In another aspect, the methods of the invention involve observing the level of one or more of PLCD4 (including reference to any isoform of PLCD4, any precursor of PLCD4, any fragment of PLCD4), and/or any one or more nucleic acid encoding one or more of the foregoing.

The inventors contemplate that a decrease in the level (for example, a decrease in the level of expression) of PLCD4 (including reference to an isoform, a precursor or a fragment thereof) and/or a nucleic acid encoding same is linked to a deleterious effect on productivity and/or worth of an animal. An animal, cell or embryo is identified as carrying a biological marker linked to a deleterious effect in productivity and/or worth of an animal where there is a decrease in the expected level of PLCD4 (including reference to an isoform, a precursor or a fragment thereof) and/or a nucleic acid encoding same. In one embodiment, the inventors contemplate that any decrease in the level indicates the animal, cell or embryo carries a biological marker linked to a deleterious effect in productivity and/or worth of an animal. In one embodiment, the decrease is at least approximately 20%, at least approximately 30%, at least approximately 40% or at least approximately 50% compared to an animal or animals that do not carry a biological marker linked to a deleterious effect in productivity and/or worth of an animal (including reference to the same animal if it did not have such biological marker).

In certain embodiments, the methods of this aspect of the invention will involve taking a sample from an animal, observing the level (in one embodiment the level of expression) of PLCD4 (including reference to an isoform, a precursor and/or a fragment thereof) or a nucleic acid encoding same, and comparing the level against one or more standard. In one embodiment, a difference in the level between the sample and the one or more standard can be used to identify whether the animal, cell or embryo carries a biological marker linked to a deleterious effect in productivity and/or worth of an animal.

In one embodiment, a standard comprises a level of PLCD4, a precursor thereof, an isoform thereof a fragment thereof, and/or a nucleic acid encoding any one or more thereof which is associated with an animal or animals which is/are known not to carry a biological marker linked to a deleterious effect on productivity and/or worth of an animal. In one embodiment, where an animal, cell or embryo has a lower level of PLCD4, a precursor thereof, an isoform thereof, a fragment thereof, and/or a nucleic acid encoding any one or more thereof compared to the standard it is identified to carry a biological marker linked to a deleterious effect on productivity and/or worth of an animal. In certain embodiments of the invention, an animal, cell or embryo is rejected if it has a lower level of PLCD4, a precursor thereof, an isoform thereof, a fragment thereof, and/or a nucleic acid encoding any one or more thereof compared to the standard. In one embodiment, where the animal, cell or embryo has substantially the same and/or a higher level of PLCD4, a precursor thereof, an isoform thereof, a fragment thereof, and/or a nucleic acid encoding any one or more thereof compared to the standard it is identified not to carry a biological marker linked to a deleterious effect on productivity and/or worth of an animal. In certain embodiments of the methods of the invention, an animal, cell or embryo is selected if it has substantially the same or a higher level of PLCD4, a precursor thereof, an isoform thereof, a fragment thereof, and/or a nucleic acid encoding any one or more thereof compared to the standard.

In one embodiment, the standard comprises a level of PLCD4, a precursor thereof, an isoform thereof, a fragment thereof, and/or a nucleic acid encoding any one or more thereof which is associated with an animal or animals which is/are known to carry a biological marker linked to a deleterious effect on productivity and/or worth of an animal. In one embodiment, where an animal, cell or embryo has a higher level of PLCD4, a precursor thereof, an isoform thereof, a fragment thereof, and/or a nucleic acid encoding any one or more thereof compared to the standard it is identified not to carry a biological marker linked to a deleterious effect on productivity and/or worth of an animal. In one embodiment, where the animal, cell or embryo has substantially the same or a lower level of PLCD4, a precursor thereof, an isoform thereof, a fragment thereof, and/or a nucleic acid encoding any one or more thereof compared to the standard it is identified to carry a biological marker linked to a deleterious effect on productivity and/or worth of an animal. In certain embodiments of the methods of the invention, an animal, cell or embryo is selected if it has a higher level of PLCD4, a precursor thereof, an isoform thereof a fragment thereof, and/or a nucleic acid encoding any one or more thereof compared to the standard. In certain embodiment of the methods of the invention, an animal, cell or embryo is rejected if it has substantially the same or a lower level of PLCD4, a precursor thereof, an isoform thereof a fragment thereof, and/or a nucleic acid encoding any one or more thereof compared to the standard.

In practise, the method of this aspect of the invention may comprise using two or more standards. For example, it may comprise measuring the level of a PLCD4, a precursor thereof, an isoform thereof, a fragment thereof, and/or a nucleic acid encoding any one or more thereof across both one or a pool of wild-type animals and one or a pool of animals known to carry an alteration which disrupts PLCD4. The samples from the animals to be tested would then be compared with the pooled values to determine whether they are indicative of an animal carrying an alteration or indicative of a wild-type animal.

Where it is identified that an animal does not carry a biological marker in accordance with the invention, the animal may be selected for production purposes, inclusion in a herd or breeding programme, for example. Where it is identified that an animal does carry a biological marker in accordance with the invention, the animal may be rejected for production purposes, inclusion in a herd or breeding programme, for example. Where it is identified that a cell or embryo does not carry a biological marker in accordance with the invention, the cell or embryo may be selected for use in breeding or cloning processes, for example. Where it is identified that a cell or embryo does carry a biological marker in accordance with the invention, the cell or embryo may be rejected for use in breeding or cloning processes, for example.

PLCD4 (including reference to precursors, fragments and/or isoforms thereof) and nucleic acids encoding same may be detected and the levels thereof compared to a standard using any one or a combination of techniques which are of use in identifying, quantifying and/or highlighting differential levels or expression of one or more proteins. Such techniques will be readily appreciated by persons of ordinary skill in the art to which the invention relates. However, by way of example, the levels of PLCD4 (including reference to one or more precursors, fragments and/or isoforms thereof) may be measured using protein purification methods, immunological techniques, separation of proteins based on characteristics such as molecular weight and isoelectric point including gel electrophoresis (for example, PAGE, including 2D PAGE) and microfluidics-based technologies as for example in gel-free protein separation techniques, and mass spectrometry (MS) utilizing isobaric label based MS such as iTRAQ or label-free approaches such as multiple reaction monitoring (MRM).

Appropriate immunological techniques include enzyme linked immunosorbent assay (ELISA) (sandwich ELISA, double sandwich ELISA, direct ELISA, microparticle ELISA), radioimmunoassay (RIA), immunoprecipitation, Western blotting, immunohistochemical staining, antibody arrays, or agglutination assays. Protocols for carrying out such techniques are readily available; for example, see “Antibodies a Laboratory Manual”, Cold Spring Harbor Laboratory Press (1988).

Antibodies of use in such immunological techniques may be purchased commercially or produced according to standard methodology in the art having regard to the nature of the proteins to be tested. For example, polyclonal antibodies and monoclonal antibodies may be produced in accordance with the procedures described in the text “Antibodies a Laboratory Manual” (Cold Spring Harbor Laboratory Press, 1988) using one or more of the proteins or a fragment thereof as antigen. Preferably monoclonal antibodies are used.

Nucleic acid-based techniques of use in determining the level of a nucleic acid (for example cDNA levels) may include differential display procedures, Northern Blotting, competitive PCR, quantitative reverse transcriptase polymerase chain reaction (qRT-PCR), microarray analysis, and RNA sequencing. Persons skilled in the art to which the invention relates will readily appreciate methodology for performing these techniques.

Nucleic acids, such as oligonucleotide probes and primers, of use in detecting expression levels of proteins in accordance with the invention (for example using Northern blotting or competitive PCR) will be readily appreciated by skilled persons having regard to the information contained herein and any published amino acid and/or nucleic acid sequence information for PLCD4. The nucleic acids will be capable of hybridising in a specific manner to an mRNA or cDNA associated with PLCD4 and in the case of primers they will be capable of priming a PCR or like reaction.

Mass spectroscopy techniques of use in the invention are described for example in “Proteins and proteomics-A laboratory manual” (RJ Simpson, Cold Spring Harbour Laboratory Press (2002).

The difference in the levels of PLCD4 (including reference to a fragment, precursor and/or isoform) or a nucleic acid encoding same in a sample versus a standard may be compared using standard technology having regard to the method employed to detect the protein or nucleic acid. For example, colorimetric and fluorometric techniques may be used in which a detection molecule (such as an antibody or nucleic acid probe or primer) is labelled with a molecule which can be visualised by the naked eye or otherwise detected using a spectrophotometer, or fluorometer for example. Alternatively, detection molecules could be labelled with radio-isotopes. Incorporating labels into nucleic acids during PCR amplification where it is employed (as opposed to labelling a detection molecule such as a probe or primer), is also contemplated.

Methods for labelling molecules and subsequently measuring the intensity of signals generated will be known to those of skill in the art to which the invention relates.

It should be appreciated that in addition to analysing samples and standards, the methods of the invention may include the testing of one or more positive or negative control samples to ensure the integrity of the results. For example, one could include a sample containing no protein/nucleic acid and one or more samples containing a known level of protein/nucleic acid so that results can be calibrated across different runs of the method.

The sample may be processed prior to analysing PLCD4 (including reference to an isoform, precursor and/or fragment) and/or a nucleic acid encoding same to facilitate analysis of the proteins or nucleic acids. Skilled persons will readily appreciate appropriate processing steps and techniques suitable for performing them.

In one embodiment, high abundance proteins which have the potential to make it difficult to analyse, such as detect and/or measure the level of PLCD4 (including reference to one or more isoform, precursor and/or fragment) may be removed from the sample. For example, Top6 or Top7 depletion may be used. The sample may also be subject to proteolytic digestion. As such detection of a protein or isoform in accordance with the invention should be taken to include detection of any one or more fragments thereof. Fragments should be of a length sufficient to ensure specificity to PLCD4. Such fragments will for example be at least 8 amino acids in length, more preferably at least 10, 15 or 20 amino acids in length.

Processing steps for preparing the sample for analysis of nucleic acids encoding PLCD4 (including reference to one or more isoform, precursor and/or fragment) may include lysing cells, isolating mRNA, and generating cDNA using standard procedures such as reverse transcription-PCR as will be known in the art to which the invention relates. In one embodiment, mRNA may be observed in situ.

Skilled persons may readily appreciate other means by which the sample may be processed for use in the invention.

Activity of Proteins and Peptides

In another aspect, methods of the invention may involve observing the level of activity of PLCD4 (including reference to a precursor, isoform and/or fragment thereof).

The inventors contemplate that a decrease in the level of activity of PLCD4 (including reference to an isoform, precursor or fragment thereof) it is linked to a deleterious effect in productivity and/or worth of an animal. An animal, cell or embryo is identified as carrying a biological marker linked to a deleterious effect in productivity and/or worth of an animal where there is a decrease in the expected level of activity of PLCD4 (including reference to an isoform, a precursor or a fragment thereof). In one embodiment, the inventors contemplate that any decrease in the level indicates the animal, cell or embryo carries a biological marker linked to a deleterious effect in productivity and/or worth of an animal. In one embodiment, the decrease is at least approximately 20%, at least approximately 30%, at least approximately 40% or at least approximately 50% compared to an animal or animals that do not carry a biological marker linked to a deleterious effect in productivity and/or worth of an animal (including reference to the same animal if it did not have such biological marker).

In certain embodiments, the methods of this aspect of the invention will involve taking a sample from an animal, observing the level of activity of PLCD4 (including reference to an isoform, a precursor and/or a fragment thereof), and comparing the level against one or more standard. In one embodiment, a difference in the level between the sample and the one or more standard identifies whether the animal, cell or embryo carries a biological marker linked to a deleterious effect in productivity and/or worth of an animal.

In one embodiment, a standard comprises a level of activity of PLCD4, a precursor thereof, an isoform thereof, and/or a fragment thereof which is associated with an animal or animals which is/are known not to carry a biological marker linked to a deleterious effect on productivity and/or worth of an animal. In one embodiment, a lower level of activity of PLCD4, a precursor thereof, an isoform thereof, and/or a fragment thereof compared to the standard identifies that an animal, cell or embryo carries a biological marker linked to a deleterious effect on productivity and/or worth of an animal. In certain embodiments of the invention, an animal, cell or embryo is rejected if it has a lower level of activity of PLCD4, a precursor thereof, an isoform thereof, and/or a fragment thereof compared to the standard. In one embodiment, substantially the same and/or a higher level of activity of PLCD4, a precursor thereof an isoform thereof, and/or a fragment thereof compared to the standard identifies that an animal, cell or embryo does not carry a biological marker linked to a deleterious effect on productivity and/or worth of an animal. In certain embodiments of the methods of the invention, an animal, cell or embryo is selected if it has substantially the same or a higher level of activity of PLCD4, a precursor thereof, an isoform thereof, and/or a fragment thereof compared to the standard.

In one embodiment, the standard comprises a level of activity of PLCD4, a precursor thereof, an isoform thereof, and/or a fragment thereof which is associated with an animal or animals which is/are known to carry a biological marker linked to a deleterious effect on productivity and/or worth of an animal. In one embodiment, a higher level of activity of PLCD4, a precursor thereof, an isoform thereof, and/or a fragment thereof compared to the standard identifies that an animal, cell or embryo does not carry a biological marker linked to a deleterious effect on productivity and/or worth of an animal. In one embodiment, substantially the same or a lower level of activity of PLCD4, a precursor thereof, an isoform thereof, and/or a fragment thereof compared to the standard identifies that an animal, cell or embryo does carry a biological marker linked to a deleterious effect on productivity and/or worth of an animal. In certain embodiments of the methods of the invention, an animal, cell or embryo is selected if it has a higher level of activity of PLCD4, a precursor thereof, an isoform thereof, and/or a fragment thereof compared to the standard. In certain embodiment of the methods of the invention, an animal, cell or embryo is rejected if it has substantially the same or a lower level of activity of PLCD4, a precursor thereof, an isoform thereof, and/or a fragment thereof compared to the standard.

In practise, the method of this aspect of the invention may comprise using two or more standards. For example, it may comprise measuring the level of activity of a PLCD4, a precursor thereof an isoform thereof, a fragment thereof across both one or a pool of wild-type animals and one or a pool of animals known to carry an alteration which disrupts PLCD4. The samples from the animals to be tested would then be compared with the pooled values to determine whether they are indicative of an animal carrying an alteration or indicative of a wild-type animal.

Where it is identified that an animal does not carry a biological marker in accordance with the invention, the animal may be selected for production purposes, inclusion in a herd or breeding programme, for example. Where it is identified that an animal does carry a biological marker in accordance with the invention, the animal may be rejected for production purposes, inclusion in a herd or breeding programme, for example. Where it is identified that a cell or embryo does not carry a biological marker in accordance with the invention, the cell or embryo may be selected for use in breeding or cloning processes, for example. Where it is identified that a cell or embryo does carry a biological marker in accordance with the invention, the cell or embryo may be rejected for use in breeding or cloning processes, for example.

The level of activity of PLCD4 (including reference to isoforms, fragments and/or precursors) may be measured using standard methodology as known in the art, having regard to the function of PLCD4. By way of example, the methods used may involve one or more of the following techniques: Immunoprecipitation, Western blotting, ELISA, mass spectrometry, surface plasmon resonance, isothermal titration calorimetry, luciferase assays and reporter gene assays.

The difference in the level of PLCD4 (including reference to one or more fragment, precursor and/or isoform) activity in a sample versus a standard may be compared using standard technology having regard to the method employed to detect the activity. For example, colorimetric and fluorometric techniques may be used in which a detection molecule (such as an antibody or nucleic acid probe or primer) is labelled with a molecule which can be visualised by the naked eye or otherwise detected using a spectrophotometer, or fluorometer for example. Alternatively, detection molecules could be labelled with radio-isotopes. However, by way of example the methodology described in Perrot-Applanat et al, 1997 Mol Endo 11(8) may be used.

It should be appreciated that in addition to analysing samples and standards, the methods of the invention may include the testing of one or more positive or negative control samples to ensure the integrity of the results. For example, one could include a sample containing no protein and one or more samples containing a protein with a known level of activity so that results can be calibrated across different runs of the method.

The sample may be processed prior to analysing PLCD4 (including reference to one or more isoform, precursor and/or fragment) activity. Skilled persons will readily appreciate appropriate processing steps and techniques suitable for performing them.

In one embodiment, high abundance proteins which have the potential to make it difficult to analyse, such as detect and/or measure the level of activity of PLCD4 (including reference to one or more isoform, precursor and/or fragment) may be removed from the sample. For example, Top6 or Top7 depletion may be used. By way of further example, immunoprecipitation of the protein of interest could also be used.

Processing steps for preparing the sample for analysis of PLCD4 (including reference to one or more isoform, precursor and/or fragment) activity may include cell lysis, immunoprecipitation and preparation of cell membranes, for example. Persons skilled in the art will readily appreciate other useful techniques that may be used.

Skilled persons may readily appreciate other means by which the sample may be processed for use in the invention.

Breeding and Cloning

As mentioned herein before, the invention also provides methods for breeding and cloning animals. Methods of embodiments of the invention described herein before may be used for selecting animals (including selecting their gametes, for example) for such purposes.

Such methods may comprise identifying at least one first animal that does not carry a biological marker linked to a deleterious effect in productivity and/or worth of an animal (in one embodiment, using one or more method as described herein) and mating said animal with a second animal. In one embodiment, the method may comprise further identifying at least one second animal that is identified not to carry a biological marker linked to a deleterious effect in productivity and/or worth of an animal (in one embodiment, using one or more method as described herein). In a preferred embodiment, the mating will produce one or more offspring.

The invention also encompasses breeding methods which comprise selecting a first and/or a second gamete where it/they have been identified not to carry a biological marker linked to a deleterious effect in productivity and/or worth of an animal and fusing the first gamete and second gamete to form a zygote. In one embodiment of this aspect of the invention, the method may comprise: 1) selecting a first gamete and/or a second gamete using a method of the invention and fusing said first gamete with said second gamete to form a zygote. The method may further comprise selecting any embryo that results from this process using one or more method of the invention.

The invention also encompasses breeding methods which comprise selecting an embryo that has been identified not to carry a biological marker linked to a deleterious effect in productivity and/or worth of an animal. A selected embryo is used to breed an animal.

The invention also provides a method of cloning an animal comprising selecting a cell for cloning where it has been identified not to carry a biological marker linked to a deleterious effect in productivity and/or worth of an animal. A selected cell is used to clone an animal.

In certain embodiments, one or more methods of the invention (such as those for selecting or rejecting one or more cell or embryo) may be used to identify and select appropriate gametes and embryos for these breeding methods. For example, a method of the invention may comprise selecting a gamete, embryo or cell identified not to have one or more biological marker linked to a deleterious effect in productivity and/or worth of an animal.

The invention also provides methods for determining whether or not one or more cell or embryo is suitable for being used to produce an animal (for example, using breeding or cloning methods) which does not carry a biological marker linked to a deleterious effect in productivity and/or worth of an animal.

In one embodiment, the breeding and/or cloning methods of the invention further comprise transferring one or more embryo to a gestational carrier, in accordance with any number of techniques known in the art.

In the breeding methods of the invention, animals may be mated using any appropriate methods including naturally, artificial insemination or IVF. In one embodiment, individual gametes may be selected for use in the process. In one embodiment, gametes may be selected using a method of the invention; for example, a method of the invention may be used to identify animals that do not carry a biological marker linked to a deleterious effect in productivity and/or worth and gametes from those animals selected for use in a breeding program or process or gametes may be tested in accordance with the invention and then selected for use in a breeding program or process. In one particular embodiment, a method of selecting or rejecting one or more animal (according to the second, ninth and/or sixteenth aspects of the invention described herein before, for example) could be used to select the first and/or second animal and their gametes used in IVF. In another embodiment, a method of selecting or rejecting one or more cells (according to the second, ninth and/or sixteenth aspects of the invention described herein before, for example) could be used to select a first and/or second gamete and selected gametes used in IVF. Following selection of male and female gametes, the female gamete is fertilised in vitro. At the relevant time, one or more embryo is transferred to a gestational carrier.

In one embodiment, in vitro fertilisation of a female gamete may occur and then a method of the invention used to determine whether or not an embryo has a desired genotype/phenotype and should be selected or rejected for further use in a breeding programme. This might occur where individual gametes, or animals from which they have been obtained or derived, have not been tested to identify whether or not they carry a biological marker linked to a deleterious effect in productivity and/or worth prior to fertilisation (accordingly, the invention should be taken to include methods of breeding where the first and/or second animal and/or gametes are not selected on the basis of such a test, but a resulting embryo or offspring is tested and selected). Alternatively, a method of the invention could be used where the individual gametes or animals from which they have been obtained or derived have been tested and selected on the basis of having a desirable genotype/phenotype, for quality control purposes or to double check that the resulting embryos have the same desirable genotype/phenotype.

Optionally, following mating of the animals, one or more method of the invention may be used to determine whether or not any offspring has or may be inferred to have the desired characteristics. Such testing may occur at any time during the life of the offspring, including before birth; by way of example only, testing an embryo, a foetus, amniotic fluid, placenta, maternal blood, at birth.

In certain embodiments of the invention, gametes are fused to form a zygote. Methods for fusing gametes are known in the art.

In certain cases, cloning may be used to generate an animal. In such cases, the method may comprise selecting at least one first animal that has been identified not to carry a biological marker linked to a deleterious effect in productivity and/or worth (in one embodiment, using one or more method as described herein) and using the nucleus or chromatin from one or more cell of that animal in a cloning procedure (such as somatic cell nuclear transfer, chromatin transfer techniques, and embryo splitting). Such cloning methods are described, for example, in Bovine somatic cell nuclear transfer Ross P J and Cibelli, J B 2010. Methods in Molecular Biology 636: 155-177. At the relevant time during the cloning procedure, one or more embryo will be transferred to a gestational carrier.

In certain embodiments, a cloning procedure may utilise a cell derived from a cell line. In certain embodiments, a method of the invention may be used to select such a cell which is, or cell line whose cells are, capable of being used to generate an animal who does not carry a biological marker linked to a deleterious effect in productivity and/or worth. In one embodiment, the cell line may be an embryonic cell line.

One or more cell of use in cloning may be selected using a method of the invention. Following selection of one or more cells a cloning procedure can be conducted. In one embodiment, a method of the invention may be used to identify animals that do not carry a biological marker linked to a deleterious effect in productivity and/or worth and cells from those animals selected for use in a cloning process. Similarly, a method of the invention may be used to identify cells from a cell line which do not carry a biological marker linked to a deleterious effect in productivity and/or worth and may be of use in generating an animal that does not carry a biological marker linked to a deleterious effect in productivity and/or worth. For example, methods of the first, eighth, fifteenth and/or other aspects of the invention may be used for such purposes. Methods of the invention could also be used to identify animals whose cells could be used to generate cell lines for cloning purposes.

In one particular embodiment, a method of selecting or rejecting one or more animal (according to the second, ninth and/or sixteenth aspects of the invention described herein before, for example) could be used to select an animal for cloning. In another embodiment, a method of selecting or rejecting one or more cells (according to the second, ninth and/or sixteenth aspects of the invention described herein before, for example) could be used to select one or more cells of use in cloning.

Optionally, at various stages during the cloning procedure, one or more method of the invention may be used to identify whether or not any cloned animal carries a biological marker linked to a deleterious effect in productivity and/or worth. Such testing may occur at any time during the life of the cloned animal. By way of example only, testing of a blastocyst, an embryo, a foetus, amniotic fluid, placenta, maternal blood, at birth.

In addition, a cloning method of the invention may involve selecting desirable cells without testing those cells or the animals or cell line from which they came for the presence or absence of a biological marker linked to a deleterious effect in productivity and/or worth. The cloning procedure can be initiated and then a method of the invention used to determine whether an embryo, foetus or animal resulting from the cloning procedure has a relevant biological marker in accordance with the invention and an embryo, foetus or animal selected where it has a desirable genotype/phenotype.

The breeding and cloning methods of the invention may involve subjecting one or more cell, zygote, embryo and/or feotus, for example, to any one of a number of standard growth and/or gestation methods.

In one embodiment, if an animal, cell or embryo is identified to be heterozygous for a biological marker linked to a deleterious effect on productivity and/or worth of an animal, it is not selected for the purposes of breeding or cloning. This will help avoid the possible mating of carriers of the deleterious effect on productivity and/or worth during current or future breeding programmes, for example.

Forming a Herd

The invention also provides methods for forming a herd of animals. Such methods comprise selecting or rejecting an animal based on whether or not it carries a biological marker linked to a deleterious effect on productivity and/or worth. In one embodiment, the method comprises selecting or rejecting an animal on the basis of the results of a method of the first, second third, eighth, ninth, tenth, fifteenth, sixteenth and/or seventeenth aspect of the invention described herein, and forming a herd of selected animals. In certain embodiments, methods of the invention which involve selecting or rejecting one or more cells may also be used to select one or more animals for inclusion in a herd. In certain embodiments, where an animal is identified to have a biological marker linked to a deleterious effect on productivity and/or worth it may be rejected and not selected for inclusion in the herd. Where an animal is identified not to have a biological marker linked to a deleterious effect on productivity and/or worth, it may be selected for inclusion in the herd.

In one embodiment, an animal that is or has been identified as being heterozygous for a biological marker linked to a deleterious effect on productivity and/or worth of an animal is chosen for inclusion in a herd. In a preferred aspect, a heterozygous animal is not chosen for inclusion in a herd.

The invention should also be taken to include a herd formed by the methods described herein.

The herd of animals may be formed for any desirable reason. However, by way of example only, it may desirable to form a herd for: beef farming; milk production.

Nucleic Acids, Peptides, Antibodies

The invention also provides isolated nucleic acids carrying one or more genetic marker of the invention. In one embodiment, the isolated nucleic acid emcompasses a genetic variation at a position corresponding to position 107313998 on chromosome 2 of Bos taurus. In one embodiment, the isolated nucleic acid includes the genetic marker 107313998A and/or a marker in linkage disequilibrium therewith reside are encompassed by the invention. In one embodiment, the nucleic acid comprises or consists of the sequence of SEQ ID No. 2, or is a functionally equivalent variant thereof. In another embodiment, the nucleic acid comprises or consists of the sequence of SEQ ID No. 6 or is a functionally equivalent variant thereof.

The invention also encompasses nucleic acids which can hydridise, preferably under stringent conditions (as herein before described), to a region of a PLCD4 gene in which the genetic marker 107313998A resides. Such nucleic acids may be used as probe or primers or otherwise in analysis of genetic markers of the invention, as herein before described.

In one embodiment, the invention provides nucleic acids with are complementary to or can hydridise (preferably under stringent conditions) to a nucleic acid of SEQ ID No. 2 or SEQ ID No. 6.

Nucleic acids of the invention may have 100% sequence identity, homology or complementarity to the relevant region of a PLCD4 gene, but may also have some sequence variation. For example, nucleic acids of the invention may have approximately 80%, approximately 90/, approximately 95% or approximately 99% sequence identity, homology or complementarity.

The nucleic acids may be of any appropriate length. In one embodiment, they are at least 4 nucleotides in length, or at least 10, 20, 30, 40, 50, 60, 70, 80 or more nucleotides in length.

By way of example only, nucleic acids of the invention include DNA, mRNA and cDNA.

The invention also provides peptides or proteins encompassing an amino acid variation associated with one or more genetic marker of the invention. These may be of any appropriate length. In one embodiment, they are at least 4 amino acids in length, or at least 10, 20, 30, 40, 50, 60, 70, 80 or more amino acids in length. In one embodiment, the peptide comprises an amino acid variation at a position corresponding to position 326 of PLCD4 (for example, a Thr amino acid at this position). In one embodiment, a peptide of the invention comprises or consists the amino acid sequence of SEQ ID No. 4 or is a functionally equivalent variant thereof. In another embodiment, the invention provides an isoform, precursor or fragment of a peptide comprising or consisting the amino acid sequence of SEQ ID No. 4 or a part thereof.

In another embodiment, the invention provides one or more antibodies which bind to a peptide or protein encompassing the amino acid variation associated with one or more genetic marker of the invention. In one embodiment, the one or more antibodies are monoclonal antibodies. In another embodiment, the one or more antibodies are polyclonal antibodies.

Gene Editing Methods

As noted herein before, the identification by the inventors that an alteration in the PLCD4 gene is associated with a deleterious effect on productivity and/or worth of an animal allows for the targeted correction of an alteration in the gene having this effect using cloning and/or gene editing processes in which one or more genetic alteration is introduced into the PLCD4 gene. For example, a specific alteration may be introduced into a cell or embryo that may be used to generate an animal.

Accordingly, the invention provides any cell or embryo into which an alteration has been introduced in accordance with the invention.

The correction of an alteration in PLCD4 linked to a deleterious effect on productivity and/or worth of an animal may allow for the production of animals and more broadly the formation of herds which have desirable productivity and/or worth or an increased level of productivity and/or worth compared to an animal/animals which have an alteration linked to a deleterious effect on productivity and/or worth of an animal.

The genetic alteration introduced into the PLCD4 gene may be of any nature, including insertion of one or more nucleotide, deletion of one or more nucleotide, and/or substitution of one or more nucleotide. In one embodiment, the genetic alteration corrects a variation in the PLCD4 gene which is linked to a deleterious effect on productivity and/or worth of an animal. In one embodiment, the genetic alteration is one which corrects a variation in the PLCD4 gene which disrupts the PLCD4 gene. In on embodiment, the genetic alteration is one which increases the activity of PLCD4 compared to the activity of PLCD4 if it did not include the alteration. In one embodiment, the genetic alteration includes a genetic alteration located within a region defined by nucleotides corresponding to positions 107313997 to 107314141 of chromosome 2 of Bos taurus. In one embodiment, the genetic alteration includes a genetic alteration located at a position corresponding to position 107313998 on chromosome 2 of Bos taurus. In one embodiment, the one or more genetic alteration is a substitution of the nucleotide at the position corresponding to position 107313998 on chromosome 2 of Bos Taurus with a G. In one embodiment, the one or more genetic alteration includes substitution of an A at the position corresponding to position 107313998 on chromosome 2 of Bos Taurus with another nucleotide. In another embodiment, the one or more genetic alteration incudes substitution of an A with a G at the position corresponding to position 107313998 of chromosome 2 of Bos Taurus. In other embodiments, the one or more genetic alteration is as described herein before.

In one embodiment, one or more cell used to generate an animal includes individual gametes, zygotes, embryos, somatic cells, cells from a cell line, for example. In one embodiment, where IVF is used, a genetic alteration may be introduced into a gamete or zygote, for example. In one embodiment, where cloning is used, a genetic alteration may be introduced into a somatic cell or cell from a cell line, for example.

Such methods may further comprise testing or screening one or more cell, embryo, or animal after a gene editing step to ensure they include the desired genetic alteration.

Any one of a number of standard methods may be used to introduce one or more genetic alteration to the PLCD4 gene in accordance with the invention. However, by way of example, TALEN or CRISPR methods may be used. With CRISPR, embryo genomes can be directly modified by injection of Cas9 mRNA and sgRNA into the fertilised egg resulting in the efficient production of animals carrying biallelic mutations in a given gene, for example. Such techniques are described for example in: Precision Editing of Large Animal Genomes, Wenfang (Spring) Tan, Daniel F. Carlson, Mark W. Walton, Scott C. Fahrenkrug and Perry B. Hackett, Adv Genet. 2012; 80: 37-97. doi:10.1016/B978-0-12-404742-6.00002-8; and, One-Step Generation of Mice Carrying Mutations in Multiple Genes by CRISPR/Cas-Mediated Genome Engineering, Haoyi Wang, Hui Yang, Chikdu S. Shivalila, Meelad M. Dawlaty, Albert W. Cheng, Feng Zhang, and Rudolf Jaenisch. Cell. 2013 May 9; 153(4): 910-918. doi:10.1016/j.cell.2013.04.025.

In one embodiment, the breeding method may involve IVF. In this example, one may first chose individual male and female gametes based on their genetic merit or otherwise, conduct gene editing methods on one or both gametes to introduce at least one desired alteration into the PLCD4 gene, and fertilise the female gamete in vitro. Alternatively, individual male and female gametes may be chosen, the female gamete fertilised in vitro, and then a gene editing method conducted on the fertilised zygote to introduce at least one desired alteration into the PLCD4 gene. It should be appreciated that one or more other alteration could also be introduced into the genome of a gamete or zygote. At the relevant time, one or more embryo may be transferred to a gestational carrier according to methods known in the art.

The methods of this embodiment may optionally comprise conducting a method of the invention described herein before to determine whether or not any cell or animal has a desired alteration. Such testing may occur at any time during the process and life of any animal. By way of example testing of a blastocyst, an embryo, a foetus, amniotic fluid, placenta, maternal blood, at birth. In one embodiment, an embryo is tested prior to transferring to the gestational carrier. In another embodiment, the animal is tested at birth.

In one embodiment, gene editing is combined with cloning. In this example, one may first chose an animal for use in cloning based on its genetic merit or otherwise. A cell from the animal may be subject to gene editing methods to introduce at least one desired alteration into the PLCD4 gene. The nucleus from such cell may then be used in known cloning processes, such as chromatin transfer, somatic cell nuclear transfer and embryo splitting. It should be appreciated that one or more other alteration could also be introduced into the genome if desired. At the relevant time, one or more embryo may be implanted into a carrier female animal for gestation according to methods known in the art.

The methods of this embodiment may optionally comprise conducting a method of the invention described herein before to determine whether or not any cloned cell or animal has a desired alteration. Such testing may occur at any time during the process and life of any animal. By way of example testing of an embryo, a foetus, amniotic fluid, placenta, maternal blood, at birth. In one embodiment, an embryo is tested prior to transferring to the gestational carrier. In another embodiment, the animal is tested at birth.

In one embodiment, the gene editing methods of this aspect of the invention are conducted for the purpose of generating (or at least increasing the likelihood of generating) an animal which does not have a biological marker linked to a deleterious effect on productivity and/or worth in accordance with the invention. In one embodiment, the gene editing methods are conducted for generating (or at least increasing the likelihood of generating) an animal having desirable worth and/or productivity.

In one embodiment, the gene editing methods are conducted for the purpose of generating (or at least increasing the likelihood of generating) an animal which is suitable for production and/or breeding purposes.

In one embodiment, the methods are conducted for the purpose of generating (or at least increasing the likelihood of generating) a cell or embryo which may be used to generate an animal which does not have a biological marker linked to a deleterious effect on productivity and/or worth or an animal having desirable worth and/or productivity. In one embodiment, the method is conducted for the purpose of generating (or at least increasing the likelihood of generating) a cell or embryo of use in generating an animal which is suitable for production and/or breeding purposes.

In one embodiment, the methods of this aspect of the invention provide means for generating a cell or embryo which is of use in a method for generating an animal, the method comprising at least the step of introducing a genetic alteration to the PLCD4 gene of the cell or embryo. The invention also provides cells or embryos generated using a gene editing method of the invention and which are capable of use in a method of generating a non-human animal.

Kits

The invention also relates to kits which are of use in a method of the invention.

In one embodiment, the kit comprises at least one or more reagents suitable for analysis of one or more biological marker in accordance with the invention. Reagents suitable for analysis of one or more of the markers include one or more nucleic acid probes and/or primers and one or more antibodies, as herein before described. Skilled persons will readily appreciate other appropriate reagents suitable for detecting or observing the biological markers and having regard to the various techniques mentioned herein before.

Reagents of use in processing samples for analysis may also be contained in the kits of the invention. The kits may also comprise one or more standard and/or other controls including nucleic acids, peptides or proteins whose sequence at a particular position is known. Further, kits of the invention can also comprise instructions for the use the components of the kit as well as printed charts or the like that could be used as standards against which results obtained from test samples could be compared. Reagents may be held in any suitable container.

### EXAMPLES

The inventors have identified a genetic variation on chromosome 2. The name of the gene that this variation is in is called PLCD4 (phospholipase C delta 4)) and denoted with locus tag of NM_001046489.2.

The variation is a nucleotide substitution at position 107313998 (107313998 G>A) on chromosome 2 of Bos taurus. This results in a missense variant of PLCD4. The amino acid substitution is Ala to Thr at amino acid position 326 of the protein. The data generated and observations made by the inventors indicates that the presence of the variation (107313998A) results in a decrease in milk production traits, liveweight, and TOP (traits other than production) traits in an animal. In particular, the inventors note a significant decrease in liveweight, protein yield, milk yield, fat yield, stature, body condition score, rump width, capacity, dairy conformation, overall opinion and adaptability, and a significant increase in the milking speed.

Sequence information for PLCD4 is provided herein after:

SEQ ID NO. 1: >bosTau8_refGene_NM_001046489 range=chr2:107297620-107322322. Genomic sequence (plus strand) indicating the reference form of the PLCD4 gene based on the UMD3.1 genome assembly and NM_001046489.2 annotation.

SEQ ID No. 2: >bosTau8_refGene_NM_001046489 range=chr2:107297620-107322322. Genomic sequence (plus strand) indicating the alternate (mutant) form of the PLCD4 gene.

SEQ ID No. 3: Protein sequence indicating the reference (wild type) form of PLCD4. NCBI Reference Sequence: NP_001039954.1 1-phosphatidylinositol 4,5-bisphosphate phosphodiesterase delta-4 [Bos taurus].

SEQ ID No. 4: Protein sequence indicating the alternate (mutant) form of PLCD4. NCBI Reference Sequence: NP_001039954.1 1-phosphatidylinositol 4,5-bisphosphate phosphodiesterase delta-4 [Bos taurus].

SEQ ID No.5: Sequence representing the wild type gene mRNA transcript of PLCD4 (Refseq ID NM_001046489.2).

SEQ ID No.6: Sequence representing the predicted mutant mRNA transcript of PLCD4 (based on Refseq ID NM_001046489.2).

Research and Results

The variant described was identified by applying recessive inheritance models in genome wide association mapping studies of various phenotypic traits. Association analyses were conducted using imputed whole genome sequence data, comprising a ‘full-resolution’ analysis that has the potential to allow mapping of causal mutations directly.

Genome wide association mapping was conducted using PLINK software (vs1.9), using 65,500 animals measured for the traits referenced in Table 1. Analyses were conducted assuming a recessive mode of inheritance, testing >16.2M imputed whole genome sequence variants per trait. Prior to association analysis, phenotypes were adjusted using covariates calculated to address population structure in the mixed breed cattle population, applying the identity by state and multidimensional scaling procedure implemented in PLINK to produce 1000 covariates based on markers from the Illumina SNP50k SNP chip platform. Covariates were fitted as fixed effects using JMP software, with phenotypic residuals subsequently used as input into the recessive association models.

In the analysis of >16.2 million sequence variants with body weight (aka ‘liveweight’), the predicted deleterious missense SNP in the PLCD4 gene was found to be the most significantly associated variant on chromosome 2, and one of the most significant genome-wide. Subsequent testing of the variant in conjunction with other traits also revealed profound associations with many other phenotypes, where individuals homozygous for the minor allele chr2:107313998A generally showed significant reductions in production yields (Table 1; bolded=P<0.05).

Descriptions of many of the traits affected can be found in the publication “Evaluation system for traits other than production (TOP) for dairy cattle in New Zealand”, Advisory Committee on Traits Other than Production, August 2014, which can be accessed, for example, via the Dairy NZ website; dairynz.co.nz/media/1205535/TOP_Booklet_A4.pdf.

These substantial reductions in body weight, stature, milk production traits, and other traits such as capacity are undesirable and negatively impact the animals productivity and worth, for example. Given the diversity of traits affected, and the recessive mode of inheritance, the mutation likely causes some form of an as yet uncharacterised syndrome.

The inventors observed that the frequency of the chromosome 2 position 107313998A variant was highest in Holstein-Friesian animals.

TABLE 1

| | | | | | SD from |
|---|---|---|---|---|---|
| GENOTYPE PLCD4 | A/A | A/G | G/G | P-value REC | population mean |
| Liveweight | −58.21 | −0.5587 | 0.04781 | 1.88E−30 | 2.6 |
| Stature | −1.108 | −0.01035 | 0.0009032 | 6.17E−15 | 1.7 |
| Body Condition Score | −0.3692 | 0.009879 | −0.0003786 | 6.16E−06 | 1.0 |
| Rump Width | −0.4283 | −0.001976 | 0.0002457 | 0.00422 | 0.63 |
| Rump Angle | 0.1994 | 0.03213 | −0.001709 | 0.1368 | |
| Protein Yield | −31.35 | −0.3638 | 0.02902 | 1.38E−22 | 2.2 |
| Milk Yield | −92.08 | −0.153 | 0.1091 | 9.61E−22 | 2.1 |
| Fat Yield | −36.04 | −0.2121 | 0.02262 | 1.61E−16 | 1.8 |
| Somatic Cell Count | 48.39 | −0.3335 | 0.001819 | 0.3648 | |
| Milking Speed | 0.5437 | 0.003894 | −0.0003995 | 0.00653 | 0.60 |
| Udder Support | 0.00204 | 0.01144 | −0.0005853 | 0.9914 | |
| Udder Overall | 0.1253 | 0.005676 | −0.0003323 | 0.5258 | |
| Front Udder | 0.1358 | 0.008047 | −0.000457 | 0.5043 | |
| Front Teat | 0.1597 | 0.0192 | −0.001035 | 0.2213 | |
| Rear Udder | −0.04997 | 0.01464 | −0.0007311 | 0.7936 | |
| Rear Teat | −0.336 | 0.02861 | −0.001348 | 0.0559 | |
| Survival | 0.3236 | 0.03516 | −0.001741 | 0.7049 | |
| Shed Temperament | −0.4462 | −0.0003855 | 0.0001825 | 0.07356 | |
| Legs | −0.2377 | −0.009588 | 0.0005701 | 0.05228 | |
| Gestation Length | 1.152 | 0.02306 | −0.001651 | 0.1916 | |
| Fertility | 0.03231 | 0.003693 | −0.000183 | 0.7026 | |
| Capacity | −1.512 | −0.01646 | 0.001352 | 4.07E−17 | 1.9 |
| Dairy Conformation | −1.563 | 0.006263 | 0.0002083 | 1.27E−17 | 1.9 |
| Overall Opinion | −1.442 | 0.01391 | −0.0001937 | 1.58E−09 | 1.4 |
| Adaptability | −0.7856 | 0.02032 | −0.0007648 | 0.002828 | 0.70 |

To attempt to validate the effects demonstrated for the PLCD4 mutation observed in the discovery population of 65,500 animals, the inventors conducted another association analysis in a separate population of 53,799 lactating cows. This analysis focused on evaluation of the Milk Yield trait only, since fewer phenotypic measures were available on this cohort of animals. To provide the most robust validation analysis possible, an alternative association model was also implemented for this study, where a mixed linear model was fitted using GCTA software (version 1.91.1). This model incorporated a genomic relationship matrix (GRM) to account for population structure, calculated using 34,738 markers from the Illumina SNP50k platform. Under expectation of a recessive mode of inheritance, genotypes for the PLCD4 107313998 variant were also recoded to merge the heterozygote and major allele homozygous classes prior to analysis. Association testing of the effect of the 107313998 variant on milk yield revealed a large, negative effect for the minor allele homozygote class (i.e. 107313998AA). This was a highly significant effect (P=5.08e-11), supporting the results of the original analysis in the discovery population, and confirming the deleterious impact of the PLCD4 107313998A allele.

Bioinformatic Analysis

Bioinformatic analysis of the variant shows that the mutation is likely deleterious, assigned a SIFT score of 0.02 (where 1=Benign, and 0=Deleterious), and a PolyPhen-2 score of 0.994 (where 1=Probably Damaging, and 0=Benign). SIFT is described herein before. PolyPhen-2 is described, for example, in “Predicting Functional Effect of Human Missense Mutations Using PolyPhe-2”, Adzhubei et al, Curr Protoc Hum Genet, 2013 January; found at: ncbi.nlm.nih.gov/pmc/articles/PMC4480630/.

Further Animal Studies

The results of the genome scan implicating the PLCD4 107313998A allele as having a deleterious impact was also validated in a population of 635 calves. Here, animals potentially homozygous for this variant (107313998AA) were identified from pedigree records, where young animals that had a carrier sire, and carrier maternal grandsire from a national animal information database were considered as candidates. A subset of these animals were then tissue sampled and genotyped for the PLCD4 variant using a custom Taqman assay (Thermo Fisher Scientific). Animals were also weighed on an electronic scale during tissue sampling. These 635 calves had a mean age of 270 days and mean weight of 197 kgs. A total of 10 animals tested homozygous for the PLCD4 107313998A variant. To test for association between genotype and animal body weight, a standard least squares model was fitted using JMP software (v12), coding PLCD4 107313998 genotype as a recessive class variable (i.e. 107313998AA versus 107313998AG/GG). These models incorporated fixed effects for farm of origin, Holstein-Friesian breed proportion, and age at weighing, and also included sire as a random effect. This analysis demonstrated a strong effect of predicted sign for PLCD4 genotype (P=7.67×10−17), with homozygous 107313998AA animals 54.8 kg lighter than animals carrying one or more 107313998G alleles. This corresponded to a >25% reduction in body weight, demonstrating the profound impact of the PLCD4 107313998 genotype.

Comparison to Model Organism Peptides

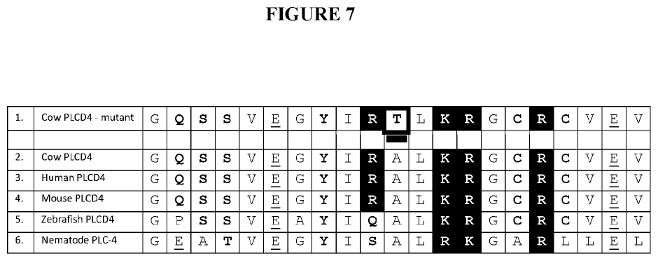

Multiple protein sequence alignment using a database of model organism peptides shows strong evolutionary conservation of the variant for the homologous proteins, with the Ala residue conserved between Cow, Human, Mouse, Zebrafish, and Nematode: see FIG. 7.

A person skilled in the art would understand the strong conservation means that variations/mutations in the region could have major functional impacts on the function of the PLCD4 gene.

General Information

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1-23. (canceled)

24. A method for identifying whether or not a non-human animal, cell or embryo carries a genetic marker linked to a deleterious effect on productivity and/or worth of an animal or carries a biological marker linked to a deleterious effect on productivity and/or worth of an animal, the method comprising
a) at least the step of analysing a nucleic acid of said animal, cell or embryo to identify whether or not it includes a genetic variation which disrupts the PLCD4 gene and/or a genetic marker in linkage disequilibrium therewith, and/or
b) at least the step of analysing a PLCD4, a precursor thereof, an isoform thereof and/or a fragment thereof of said animal, cell or embryo to identify whether or not it includes a variation which disrupts PLCD4;
wherein where the nucleic acid includes a genetic variation which disrupts the PLCD4 gene and/or a genetic marker in linkage disequilibrium therewith, or where the animal, cell, or embryo includes a variation which disrupts PLCD4, the non-human animal, cell or embryo is identified to carry a genetic marker linked to a deleterious effect on productivity and/or worth of an animal.

25. A method for selecting or rejecting a non-human animal, cell or embryo, the method comprising selecting the non-human animal, cell or embryo where it has been identified not to carry a genetic marker or a biological marker linked to a deleterious effect on productivity and/or worth of an animal according to a method of claim 24.

26. The method of claim 25 wherein, the animal, cell or embryo is selected if it does not have a genetic variation which disrupts the PLCD4 gene and/or a genetic marker in linkage disequilibrium therewith, or if it does not have a variation which disrupts PLCD4.

27. The method of claim 24, the method comprising the additional step of: estimating the worth of the animal and/or its offspring on the basis of the presence or absence of a genetic variation which disrupts the PLCD4 gene and/or a genetic marker in linkage disequilibrium therewith, or of a variation which disrupts PLCD4.

28. A method of cloning or generating a non-human animal, the method comprising
at least the step of selecting a non-human cell that has been identified not to have a genetic variation which disrupts the PLCD4 gene and/or a genetic marker in linkage disequilibrium therewith, and/or
at least the step of introducing a genetic alteration to the PLCD4 gene of a cell used to generate the animal; and
cloning or generating the non-human animal from the cell.

29. The method of claim 28 wherein the genetic alteration introduced to the PLCD4 gene corrects a variation in the PLCD4 gene which is linked to a deleterious effect on productivity and/or worth of an animal.

30. The method of claim 24 wherein the genetic variation which disrupts the PLCD4 gene and/or a genetic marker in linkage disequilibrium therewith, results in a decrease in the level and/or activity of PLCD4.

31. The method of claim 24 wherein the genetic variation which disrupts the PLCD4 gene and/or a genetic marker in linkage disequilibrium therewith, is a missense variant.

32. The method of claim 24 wherein the genetic variation which disrupts the PLCD4 gene is located within a region defined by nucleotides corresponding to positions 107313997 to 107314141 of chromosome 2 of Bos taurus (which defines exon 8).

33. The method of claim 24 wherein the method comprises at least the step of analysing a PLCD4, a precursor thereof, an isoform thereof and/or a fragment thereof to identify whether or not it includes an amino acid variation at a position corresponding to position 326 of PLCD4.

34. The method of claim 24, wherein the method comprises at least the step of observing the level and/or activity of a PLCD4, a precursor thereof, an isoform thereof, a fragment thereof, and/or a nucleic acid encoding any one or more thereof of the animal, cell or embryo.

35. The method of claim 34 wherein the method comprises the steps of:
observing the level and/or activity of a PLCD4, a precursor thereof, an isoform thereof, a fragment thereof, and/or a nucleic acid encoding one or more thereof in an animal, cell or embryo; and,
comparing the level and/or activity of the PLCD4, a precursor thereof, an isoform thereof, a fragment thereof, and/or a nucleic acid encoding one or more thereof against a standard.

36. The method of claim 25, the method comprising selecting at least two animals that have been identified not to carry the genetic or biological marker linked to a deleterious effect on productivity or worth of an animal, and forming a herd of said selected animals.

37. The method of claim 24 wherein the deleterious effect on productivity and/or worth of an animal is a deleterious effect on one or more of protein yield, milk yield, fat yield, liveweight, stature, body condition score, rump width, milking speed, capacity, dairy conformation, overall opinion and/or adaptability.


SEQUENCE LISTING (6 sequences)

SEQ ID NO: 1 - DNA, 24703 bp, Bos taurus
tctcaggagcccttgttaaaaatgaatattcctggacctcatccttagaaactctgatctggtagatttgaggtggggctaaggaatctctgttttaagacactctacaggagacattgatataaccaggaaccactagattgatgtgttgtatgagtcttcaagctctaacattctgaaggctatgacctgtgacgggagccatgaaccaggtttgtcataattctcctccaaagaaagagagtagacaggatgaatctcttccatcctgccctcagagagactgtgctgctgctaagtcgcttcagtcatgtctgactctgtgcgaccccatagacggcagccctcgaggctcccccgtccctgggattctccaggcaagaacactggagtgggttgccatttccttctccaatgcatgaaagtgaaaagtgaaagtgaagtcgctcagccatgtccgactcttctcgaccccatggactacagcccaccaggccctccatccatgggattttccaggcaagagtactggagtggggtgccattgccttctccgcagagagactgtagacacctcgaattccactataagccacaggcaactctaccaaagtccatctccaggtgatatgaagccacctgtcagctgaggaccaaattttgcttggcaacccttggccacttcctgccctatttgtgtgtctattttctcatctgtattgtagaggtaattatctgtatcctcagggagctccacatatgtgcctagacgtaaacagttaagacccaacctctctctcagtcattatcctctgattcttgcctctttctcttgctcccactcccaaatcctttttccctcctctccaaactgtacctgcctcagctgtgaccatcctccccattccaccttcgggtgccagaggaaacaccaagacctagaacacctaggttctaagttgcattctctgagtagtttgtttagtcagtctcctaggatacaggggaaaatgaagctgagcagaaggatctgttactcaggatcccaacccaagagtcccccttcctgaaccaaagtctcaagcacctttcaggggacaagaatccaagccattgcaaattagaagagaaacaaaagagcaaggagtaatgaggagaagggatgaggagaaggatgagaagtcaagagcaaagagaggctgacttaggtaagcaaataggaaggcaaagggagggtcaggagagagcaaagtgagctaggaagaaaggaaaaggaaggggtcaggtagaagagggggaaggggaagaaaataggaagaaggaaaagaaaggctgttgagtgggatatggtaggaacagaaaatgatggggtggcggggagggagaacaatgtccctcactgggtcccagctctgctttcatcggcttgctagagaaagccttttctaagggtagtctcggtgaggtctctgagttggtcaaatggagacctcagttctagccccagttctgccacttaattccttaggtgactggcagttcagctagccttctgtccctttattttttctgacaaaaggattcccatacttattactgagctccatagaccgctggtggggaaacagaaagcacggggagctaagcatttcgtctcccctgtgagttgttcaaagagcggtgatgttctgtgatctggttggttaacgtacaaccttaacacaggataaatgtgtgccacttcatacgtgaagcttctgacacagccagcttggttcttctccagtgtcttctcttggagttgtatctgattacctgttttcattggaatctattgaggaatgggatgattctgcttttttttgtggtcaggagttgcctgatcctgaaagtcttgtgagaagacatggtgagatgcaaactcaatcacacgtaggatggcagagaaaagagctcctctgtcccttttctacaaaatgaagtccagctctgacatcctgtggctccctgagttgaacctagccctctatcggccacgtgtcacctagatacagttttaaaacagtgactcaagaatgggagcagggcacgccaagctctgaggttgaaagagtttgacaaaagaatataaaatcattttctaaggctggtttgagatgaggtagggctgttcctgtgcttgggactgttctcagatctcccatgtctcgacattgtggtgaggctcccagacacaggcatatggaaggtaattctgtcttctcccagggccaaagcccacccaccccagaagttcagaggagagaggaggcaaaatgttggcaaaggggattagacttcagccccaggttgaccccaacccctcttagagtctgaaactctaagacatttaatgaagatctattactttttagtcttcatagaacttttgcaaaccttggattccaagctgtgcagcaccacccagcaaagcagagatgctttcctgtcttctttccttccttcttccatccagaggtctcctctggtctcactcaggattctacgtgcagcctcagccagtaaaacctttccctcttgggactgaagctgtgttcttgttctcctgagagttgccttatctcttctgcttagactcagattggtttcctgcacattcctactcctttggaaaatcttctctaggtccttcagtcaggagagctataatttcagtgacttctacatcattcctgaagatcaccaccctgggtggctttacaggctcttcctacttaagggagatggggcactaatatgctgtgtgacctcagacaagtcacatcatgtttctggatttctgctctaacgtagggagttgaactacatgatgtttaaatggactactaactgaataattagtttaaggattcttgccttttttttttttttagatgttcaccactgacaaattgttaaacttattcagggatgttcctggcagtccaggggttaagacttcacactttcactgtaggaggcacaggttcgatccctgattggggaattaagatcccacatgctgcaaagggcagctggaaagacttcttttaagtttaaacttactaattaaaacccacaatatttctcttcaaccatggatcctttgcttatacaagagcataaagctttatgttccgtggactttgccagtttcagcatgatttgttttaagcactgagtatatttgttctaggcacaaagctaggatcaaagtggccacaaatcaaatcatccagttcttttcaaggcaagtttgttgttagctcttaggataacttcagttcagttcagtcactcagtcgtgtccaactctttgcaaccccatgaatcacagcacgccaggcttccctgtccatcaccaactcccggggtttacccaaactcatgtccaccaagtcggtgatgccatccagccatctcatcctctgtcgtccccttctcctcttgccccccaatccctcccagcatcagagtcttttccaatgaatcaactcttcgcatgaggtggccaaagtattggagtttcagcttcagcatcagtgcttccaatgaacacccaggactgatctcctttaggatggactggttggatctccttgcagtccaaggaactctcaagagtcttctccaacaccacagttcaaaagcatcaattcttcggcattcagttttctttacagtccaactctcacatccatacatgaccaccaaaataaccagtcctaaatgagcaatggcaccccactccagtactcttgcctggaaaatcccatggatggaggagcctggtgggctgcagtccatggggtcactaagagtcggacacgactgagcgacttccctttcacttttcactttcatgcattggagaaggaaacggcaacccactccagtattcttgcctgagaatcccagggacgggggagcctggtgggctgccgtctatggagtcgcacagagtcggacacgactgaagtgactcagcaacagcagcagatgagcataaaaggtaggaactctctttcagtaggtagagaaaacttgaacatgctgttctgagactatgtaacttatctggttcattaaaactgtgagaattactattatgaataagagtatgagctatactttatcatgtgaaggtagaagtggctcatttagcaaggcgcagcactaagtgtggtagaagagacaaaagaaatactctggcctagcttgtccagagctggaggatccacacaagagcatggagagggctcctggaagggaagttgagatgattctgcttggagaaggaaaggctaggatgcagagagattccaagttctgaaagggggcagccagcaagtcagtgattcctaataatggggaactgagagaggtgtggtcaacatgacctcgtggattgtagggtgttggaaaagaagagggtatttggcagaaacccctgggttggccctgtcagagatggctgtgggaaacggcagaaactctgaaatcagatatatttgggtttaaattccaagtccctgtgtgattgagacaagtttcttagcctctctaagcttcaatcactaaactcaaattagaagtgaaaagagttacaaattatgtagatgaagtagctgacacaacgcaggcagtcaaggaacactaatttccttctcttcctccctcgatctattactctaagacctcaagattaacctgaagcctcttgctgctcctcgctcctttaggtgactctggcgtcagctggtgaagcagtgggtgatggcgtatctgctgcaaggccgtgagtacccctgcagaagagggcttggggagacagcccagagacagggaggaggctggactcctgttccaaagctcctcctgggccctccaccttccctaccggctactcagtccctgcttctcctggctcagggctgcccatcaatcaagatctgctgctgatgcagaaaggcacaatgatgcgcaaggtgaggtccaaaagctggaagaagctaagattcttcagacttcaggacgatggcatgacagtctggcatgcccggcaggctggaggcagggccaagcccagctgtgagtgatgtgggtagccaggggtgggcatgtgggtggacaggaccttacaggcccagcagcttgagggcagaggagtgaccagtgtcttatgatgtgccctgttctggtctctttagtgtctttatctgagcctgtaaaatggaaattataactgtactagcctcttcagggtgttacaaggatcaaattggttatggtgtgcaaagatcttccaagagtctttgccacataataaggctcaataaatcataggttttatcttattactatcatcatcatcatcattattatagcataatctcaggtagccaactgtctagacaaaagccctggctctgccaatttctagctgagtgactttggacaagttatttaacttaagctaatataagtttcctcatctataaaatgaggattaaacagcctagtacattacaggagctcaagaacttaatgtatataaaccacctagcatgaggcctgaaacccggatcaatttagtgagttaaccattatagtgaatatttctaaatatcgtaaatgcgtcataacagtgttcctcctattggccaaacactttatgatccctctaaaaatgtgaccacaggactcacctaagagaatctactttagagacataaccagagcttacgatccacaccagccccagctgaagaagcagggggaaggatttctctggctccctcccagacatctggaaagaggaagccctcaaacctggaatgagagaactgaatttgggcgcgtcatgctttggtgtaactgcttctatcctgcaagtgtgcttccacttgtcagccccaccagtcttcctgtaattgatcaccattggattggccatggaagggaccatcccttatttaagattaggatgctgaggggctacgtcattttcatccctgtcacactgccagttggtggctggaagccaggtcttgccatttccagcacaggccccttgtcatgctgctatgaaaacaacgactcatgtggcaatctgaattccaagaatctgcatctccctgtactaaggctttgggggttttagtctatggaggctggggtccctcctcctgtggtctcagagagaagaagggggagtactggtccttggctctcagctgctgtgatttgtcataccctttgcttagggccccccactaacttcttccctggcagtctcaatctccgacgtggacacagtacgtgagggccacgagtctgagttgctgcgcaacctggcagaggagttccccctggagcagggcttcaccatcgtcttccatggccgccgctccaacctggacctggtggccaacagtgtccaggaagcccagacctggatgcaggggctccagctgttggtgggctttgtcaccaacatggaccaacaggagaggctggatcagtatcagcaggatggagtcagagtgggatccaggggtcactgaaggagccagatgctgaagggctagggacaagtaggagggggcacagggctagaggggcctgagagtccagatggggtggccagggacagcttgggctgatgtgaggctgagatactgagggaagaatggaggcctgtgatggcacagacagagtcacacagagtttggagaggaatcagggcccccaggattgggtaaagggaccgtgagaggcaaactaggagctgatcctgagagctgaacagaagatcccgtggcattaactcccatagggatggggagcaactccttagggggcttagtgggatcaactcacttcaagtttcctagaatgtattcatcttctatgctagtccattcatttattcaccatctattaagcagaatgtaagaggcactatgccaggctttagggatacaaagattaatgaggctcagatattattttagtcagcagagaaaatagaattgtgtgaataatttaatttaccagcattacagtacttagctgatatgtgggtgttcaaaggaaggagaaatacttatttctagctgagatggtcatgagaaagcttcaggagaaagtagcgtttgagctgagccttgaaaatgagtagaatttgaactgaagggaagaggaggggaaggcacttgtgtctgaggaaacatcatgaataaagacccagaggtaggaagctgaagagctgggacatcaccaagcaggggcttgctgaatctgtctgaggtacaaggtagtcatttctgtgttgagaaggctggtttggggccagatcagtggggtgaaggagatcttaatttctatgttagggaagttgggcttctttttgcaggcaacagggatgtccccaaaggcacctgagccatgcaggggcaagatagattggtgctctggggagattattccagaagctaatatggaaaatgaattggctctggggaggggactggcaacaaagacaggggagcagtcaatggatgaatgtgcacacacatctgcacacacaaacacacactcagtgcttttgccttgactcagacctggatggctgagtgattggttccagcgcggagacaaaaaccaggatggtcggatgagtttcggagaggtccaacggttattgcacctgatgaatgtggaaatggaccaggaatatgcctttcagcttttccaggtgagtcttctggggaaaggttagcagaagccaatcaaggccacattctcccttagccagaagtcctccaaccctctccagcaacacacagctgttgaatctctcctactacgccaggttcattgggaagtgtgggtaggacatggttcttacccttcagtcttgttggaaagatgactcataatcttatgaaaggtaaataacaacatgttattacatcagacctcagggacctctctgtccttggccaggatttcacaagtgcctagtacagtggaaaactccaactggcatgtgagtaaatagaaaaagggatcttctgatcttttagggacatgttgggggcatttaattctttctagttgaaatttttctccagtagcaaatggtatggccgaaattttgctttttacttcctgtcgtggcttttcctatgaacactaaatgtcaggagttctgtggaagccagcctggctccatcaataataacgcccttacaaggaaagtcaaaactgcaacatctgccgttaaagggttttttactctcctctaaaccctcttctgcagacagcagacacatcccagtctgggactctggagggagaagaatttgtagagttctataagtcattgactcagcgtccagaagtgcaggaactgtttgaaaagttttcatctgatgggcagaaactgaccctgttggaatttgtggatttcctccaagaggaacagaaggaaggagaacgggcttctgaccttgctctggaactcatcgaccgctatgagccttcagaaagtggtaagaggactagggtggtgaggcaacacacatgggggccagatgggggaccaagagacaactagccttctgcctaactaagggaaaattcagtcccccagaccccgttcccaatcatcttcatctcattcaatcactcctccagaaggagaaagccatttcctgcccacataagacaggagtctcctctgtagtgctgtatctcttaggaggaagaacattccgtaagtcccctttcctctaccacgctcatatttctcattcttcactttcacactgttcttcctgatatcaacctttgtcctcagtggaaacataaaatccttctcctcttacttttggcttctctagggcatgtgattcctttccctttagttcccttcatggttcctgtttgctgtctctttaagtgctggctctctgccctttcctggactttctccaagttccccaggtggtttttgagtcctaagtccctgagctgggcacctagtcagcccctggctgaggtggatgatgaggtagacaatcatttggctcttaacaagcagttaaaatatgccctctctgccctcaaaccttctccttgcccctttagtagataccttcctgcccttcattccttgcagagcatgaaaaacttacattattaataaaaagagggcttccctgatagctcagttggtaaagaatccacctgcaatgcaggagaccccagtttgattcctgggttgggaagatccgccggagaaaggataggctatccagtccaatattcttgggcttcccttgtggctcagctggtaaagaatcctcctgcaatgcgggagacttaggttcaatccctgggttgggaagatcccctggataaggcaaaggctacccactccagtattctggcctggagaattatatggactatatatatagtccatggggtcgcaaagagtctgacacaactgagtgacttccactttacttcactttatagctcttacattatttagtcctttgtgtgtgtgtgtgtgagtgtgtgagtgtgttagacgctcaatagtgtccaactctttgcgaacccatggactatagcccaccaggctcctctgtccatgggatttcccaggcaagaatacaggagtgggtaggcattcccttctccaagggctcttcccaactcagggataaaaccccaggtctcctgcactgcaggcagattctttaccatctgagccaccagggaagcccttagtactcactgctgctactgctgctgctaagtcgcttcagtcgtgtccgactctgtgagaccccatagacggcagcccaccaggctccgccatccctgggattctccagacaagaacaccggagtgggtttagtactcactacatccttgtaaatcaaaagcaataatctttattgggcctttgctaagcatcccataatgtgctgatatctctacatgccttttctcttctaatccttccaacaacccatggaagtagatttctattatcaactcagttgtacagaagaggaaaactgagattcagaaaaattaaatgacttgctcaaagtcaacagctaatgaaaggaggaattgtaattcaaagccagatcattgactcttacctggtgcctctcttgagaatgagaataagacatgagaggaatgtgagctggacaggtgacccaggtttcttaggtctcaattcccattccctttcattcactcattcattcattcactcactacttcagcaaatgctcactgagtacctataatgtgccaggaccaatgataggcaacagagatacagtagtaggtcagacaaggacactaccctcatggaccgtggagccaaacaaagtgttgcaatcactgattccagatttatgtgggtgcttagagaagaaaggggctgtgacgcaatcttttcagaaaactaaacagaagaggaagttgaagggttaagacaggatcctctgtttccaatgaaaaatccagcatacatcattacatttattctgcctgtgtgcatgctcagtcactcagtcgtgtccaactctttgtagcccatggactgtagcccaccaggctcctttgtccatggaattttccaggcaagaatactggagtaggttgtcatttcttactccaggggaccttcccaacccagggactggatccttgtctcttgggtttcctgcactggcaggcagattctttaccattgagccacctgggaagccccacatttattctatccaccctttttggagttctttcccttttcacttgattatcagatacaacaacactgtggctgcaaagtgtggtatcagtcaggagacagaggctcaagtccccactctgcttcttatagagtactgagttgcttaacctatcggaggctcagttttctcatttgtaaaataactggtgatcaaatgaaataaagcacatgaaaagtacctggtagctatggactttatcaatttgcaatttgagtcattacagtcaagaatgaaactgtgggatttctctggcagtccagtggttaagactttgaacttacactgaagggggcatggatttgatctctgatcgggaaactagcaccctgcacgcctcaagtctggagaaggcaatggcaccccactccagtactcttgcttggaaaatcccatggacggaggagcctggtaggctgtagcccatggggtcactaagagtcagacacgactgagcgacttcactttcacttttcactttcttgcattggagaaggaaatggcaacccactccagtgttcttgcctggagaatcccagggacgggggagcctggtgggctgccgtctatggggtcacacagagtcggacacgactgaagcgacttagcagcagcagcagcatgcctcaagtcatggccaaaatatagaaagaatgaaactttgcgcccaacttcttgccattttccaccatcgccaggccttgtcacacctggaatgaggaagctaggcatgagagtaaagctgttctgcctcccctgtagcaaagttatcctcagtgagacttcacagtccagcagtgtagggcagattctgacatctgactccctgggttcaaatcccagctccttcacttattagttatgtgacataatattatataggaggcggtgaccagaaccatcccaaagaaaaagaagtgcaagaaggcaaagttgttgtctgaggaggctttacaaatagctgagaaaagaagagaagcaaaaggcaagggagaaagggaaagatacccaactgaatgcagagtttcagagaatagaaagaagagatatgaaaaccttaagagaacagtgcaaagaagtagaggaaaacaacagaaaaagaaagactagagacctctttaagaaaagtggagatatcaagggaacatttcatgcaagaatgggcacaataaaggacagaaacagtgaagacctaacagaagcagaggagattaagaagaggtggcaagaatacacacaagaactgtacaaaaggtcttagtgatctcgatttccacgatggtgtggtcgctcacctagaacaagacatcctggagagtgaagtcaggtggaccttaggaagcattactatgaataaagatagtggaggtgatggtattccagctgagctatttaaaatcccaaaagatgatgctgttaaagtgctgcacccaatatatcagcaaatttggaaaactcagcagtggccataggactggaaaaggtcagttttcattccaatcccaaagaagggcaaagccaaagaatgttcaaactaccacacaattgcactcatttcacatgttagcaagtttttaatgctcaaaatccttcaaactaggcttcagtagtacacaacagatatacaagctgggtttaaaaaaggcagaggaaccagagatcaaattgttaacattctttggatcatagaaaaagcagtaaaggaattccagaaaaacatctacttctgtttcattgattatgccaaagtctttgactgtgtggatcacaataaactgtggaaaattcttcaagagatgggaataccagaccaccttacctgtctcctaagaaacctgtatgcaggacaaggagcaacagttagaactgtacatggaacaaggactgcttcagaattgggaaaggagtatgtcaaggctgtgtgttgtcatcctgcttatttaccttatatgcagagtatcttatgtgaaatgcgaggctggatgaatcacaagctggaatcaagattgccaggagaaatattagcaacctcaaatatacaaatgataccactctaatggcagaaagtgatgaggaacaaaacagcctcttggtgagggtgaaagaggagagtgaaaaagctggcttaaaactcaacattcaaaaaacgaagatcatggcatccagtcccatcactttgtgcaaatagagggggaaaagtggaaacaacgacaaattttatttccttgggctccaaaatcactgcagacagtgactgtgtgcgtgctaaatcacttcggtcatgtacgattctgtgcaaccctagggacggtagcctgccaggctcctctgtgcatggaactctccaggcaagaatactggagagggttgccatttccttctccaggggatcatcttgactcagggatcaaaccagggtctcttatgtctcctacagtggcaggcaggatctttaccactagccacctgacagtgactgcagccatgaaattaaaagacttgctccttggaaggaaagctgtgacaaacctagacagcatattaaagaacagagacatcactttgctgacaaaggtccgtatagtcaaacctatggtttgtccagtagtcctgtatggctgtgagagttggaccataaagaaggccgaatgcctagaaattgatgctttcgagttgtagtgctggagaagcctcttgagagtcctttggacagcaagaagatctaaccagtcaatcctaaaggaaatcaaccctgaatatttactgaaaggactaatgctgaagtttcaatactttgaccacctgacatgaaaagccgactcattagaaaagaccctggtgctgggaaagattaaaggcaggaggagaaggggatgacagaggttgagatggttggatggcatcactgactcaatggacatgagttagagcaaactctggcggacagtgaaggacagggaagcctggcatgccctagggctgcaaagagtcagacatgactgagcgactgaacaacaaccacaaatgtgacccagggtatgttacctaacctctcagggcagattatgttatatccacctgattgtgttggtgagaggattaagggagatgctgcatgttatgtccttggcaacgctcaagaaaccctcaggccatagtaggcagtagctacacagcccagggcataacacacaggtaccttggtcttggggcttcccaggtggtgttagagctgaagaacgcctgtgccaacataggaaatgtaagacatgtaggttcgatccctgggttgggaagatctcctggaggaggcatggcaaccctctccagtattcttgcctgaagaatctcgaggacagaggagcctggcaggctacagcccatagggtaacaaagagtcttcagacgtaaagcaacttggcattcatgcatgcaccttggtcttgtcactaacaagtctttgactttgatcctctctgagcctcgggtgccccatctatgaaattagtcctttggactaaatactgtgctgtccttcatggtaaccctgagccccatgtgactatttaaatttaaattacagtcggctctctgtatctacaggtgccacatcgcggatacagagggtcgactgtaccatgagattctgtgtaaggcacttgagcgtccatggattttggtatctgctatggggaaggggccggagaaggcaatggcaccccactccagtactcttgcctggagaatcccagggacaggggagcctggtaggctgcagtccatggggttgctaagagtcagacatgactgaatgacttcactttaactttttactttcatgcattggagaaggaaatggcaacccactcccgtgttcttgcctggagaatcccagggacaggggagcctggtgggctgccgtccatggggtcgcacagagtcggacatgactgcagtgacttagcagcagcagcatggggaagggggaagtcctggagccaatctcctgtgaatacaaaacagtaattcagttccacagtcacacagcaacatttcaaatgctcagtagctttatttatttagaggttatcatactgggctgtgcagaccagagaacacttccatcactgtagcaagttctgttgggcagcactggactagcatgatcttaataattccctttgtatttttatggttctctgaggatggggatgcttgaaatgacaggctggatagggcagtggggtctggaggctgggagtggacaccatttgaaaggctgggccctggagaggggtggggtgctctgagcagagagcttctcactctagccactccacaccccaggaaaactgcggcatgtgctgagcatggatggcttcctcggctacctctgctcgaaggacggagacatcttcaatccaacctgccaccccctctaccaggacatgactcaacccctgaaccactactacatcaactcctcccacaacacctacctggtgggggaccagctttgtggccagagcagcgtggagggatacatacggtgctgtggggctggggggtgaaggggtccagctcatgagatgggaaggtctgtgggaagttggaggggaattgttagttgactgcaggaattgaaatgctgttgtggggggatttgagctgaggattccttggtccctgagagacttggaggagatattgaagactggcaggagagtgctgtggaaaccctgcgggtaaacacagcttctctttcataggacatgaaattaccttggcaccagaggtcctgacaaatttattttgaacaaataaataaactcaatgttaaatgggaggcagtagggaagaggatttaaaaacatatgttctggagtcagaccatttaagatcaaccactagctccctcacttggtactctgaggtcaaatacttaacctctctctgcctcagcttccctatctgagacacagagaggattactattctgtctattctgtctagcttgtcgtgaggattcaataaggtaatttatgtgtcaataggtaataaatggtacctgactttattgtgattaaccatgcaataggtcaaatagtcaaggcatctttgtaatgtcagcagctgaactagtcgcttcaagtgcaacttccctaggaagcctagcaaggaagtaaaatgtaaatttactgagagtttgaataactcaggtgtcatataaagatatacaagcagaacaccagaaggcagtatgtgatggctccctaagggaggattgttgagtagggcctggtggagatcagtgtgagctgcaggtgtgtcccggcagacagacaggggtccacggagatgtaaattgacagtcttgatgtgtgagtgccagtgagcactgttctctccagagagggaagagggtccctatgtgaaagatcacaccttgacccctcccgtccctggggggtggggttcttgcagggccctgaagcgggggtgccgctgtgtggaggtggatatatgggatggaccaagcggggaacctattgtttaccacggacacaccctgacctcccgcatcccattcaaagatgttgtggccgctataggacagtatgccttccaggtgggagcctcaggatggaaatactggtgattagcaagaggatctgaaggaagagactctgggtctggggttggggagggatgcaggggaaagtaaaagtattagtcactcagttgtatccaactctttgtgaccccatggactgtagcccaataggctcctttatccatagaattctccaggcaagaacactggagtgggtatccattcccttcgccaggggatcttcccgacctaggtgatgtcaaatgacatatacgtaaaagagattttaattgtgaagcatcaggcaaacattaagggattattatgaataagtgttggatgtgttactagttaagtagtcatagaagtaattgctaactgtaatgattacttctaaaaacttctgtcacccacctgggagagggcggaggaggaaacagacaaactggtgggtagagctgggaggcaatagctatctggggatgaaagtctgaaagtataacgtgaacatgtctggggcactggcctggctttcccactctaagcctgaaaactcaaaggtcccataaggacctgggtcagacctggcacagatctgagatttggggtcccaagaatgacttttagatgtggggtgcctagacccaggcagatctcctggaggcctggcatggggtcagaggacttttggcaatcagggtactcagttgagtaattggcagggggtgggtggtggggtggtgggtgggggtggagctggagaatcaagaaggttataggcctcagaactctggctccagcatgccttcccctcctgaccagacgtcagactatccagtcatcttgtcgctggagaaccactgcagctgggagcagcaggagatcatcgtgcgccatctgacggagatcctgggggatcagctactgacgacagccttggatgggcagcctcccactcagctgccctcccccgaggtagggaccctgccttctagcccaggcttccctaccgggcttggctcttcctcccaggcccatctgttcccttcttctcaccctcagatggaccgtcttgctctttaatattctcagagacaactttaatttttaaaaaatcgaattcagggtgttccagtgtggcctagtggttatgattccaagctttcactgccaatccctgcttcaggaactgagatcctgcaagccgagtggcatggccaaaagaaatttaaaaaaatctaattcaacataaaactccagatatagctttctaaatttaaaaccatttgtttactttatatttcttttttcaatggaagatcttcattttttttcctgatagttcattattataggctctacaaatataaggggagattcccccaccataatctcaacctctccaataacagctgccaacattatagattttttatacacttatatagacatatatatgtatatatggttacatttttaaaaaatgtgaatgggataatcctttctatcccttttatatatatcacaaatattttttcatatatattttcttttttaacagttctatagtattaaactttatgttactgaccataatttactcaaccagtaggtggtaaaaataaccagtaaatggtaaacatttaggttgtttataatttttcatttgtacatagagctttgctcatttactctttctttgaaatggattcctaggaaaaaaattgctgggttgaaaactgcatgttgacatttttggttattattgtcaagttgccctcgtgggggggggcagggtgcagaaaaaaaccaaatttcactccaactgaacagtgtaaggggatgactacaaacaactccagtagtgagatgattcgtaatggcaaacatgaaaaaaaagtttttaaaaaacataggtctgttttattctttcaaattaactgatattatttacaaacttttttatattagagtttataatttttcttgatatataactttaaaaaatgttaaagatattaatgctttaaaaatacttttttgcagtctatttgggcaggggtggccacgctgtgcgacttgcatgacccagttcaaaaccagggactgaacccaggacacagccgtgtggagtcctaaccactggaccaccaggaaactccccttgcagtctattttttctttttgtcttcatagcctcttctgttttacaggtttttaacctttatgtagtcacctctgccaatcattttttatgtcttctgagttttcaaaggctttcctaccttgaggcttttcagaaagagtctctatcttcctctaatactctaatgttctttacatgtaaatcttgaatctatgaggaatctttacgtctcaaatgtgaggtaggggttcgcatgtattttttaccttagagatgttacaggtcggcttaaccataagaaatccctgctctatgtatcaagtagccttcctgtgggaacttccgaccacttacttgcttcctttgcctttgagatggtcctgtgagagctgcataggagctgcacgacccacaggaggggcccagagcagacaggtagacaggcccctctgtttttgtcccctaggaccttcgggggaagatccttgtgaaggggaagaagctaatgcttgaggaagaggaggaagaaccagaggctgaattggaagcagagcaggaggctaggctggacttggaggcccagctggagtctgagccccaggacctgagccctcgcagtgaggacaaaaagaaggtaggccagaagggtggtctttctgctctggcgtttgattagtcagtactacaagctgagaccccatgtgtgaagagactggagccaggcagggtggagaaccaaggagcaaaatgctccttgcctccaatgacaagaggcatgagaatccatacacagcatcatcatatagattgccatatacagacatatggcagtggctttccaacttctttgttctgtgagatctttttttttttttttaatgaaattctatatggaagctcaggtcacaaaatagatatatgcagagtcattctggttgaagtacttctttggctctctcctgggtctcccaggtgacttctgagacatctttgtactacttcccagtctctttaaatgatacttcaaaaaccactgatgtgtgcatgtgtgttgttatattaggtaagtagggcttactagagtgactgctatgacctcaactaaagtgggtttattgctagaaagtttcttggaaaaggtgagtctgaaaagggaccagaaatagccaaagaggaggagcatgaaaggttggggaagggctgccaacagtgggatcaccaagatcttgctttgaagtctgcatttctctctcttatgcacttgcttggctcagctgaatgacttctagctaattatcctgttctctctggagctgtgttgtctcacctgctgccatgcccagggatggaaatgggaattcagaaaaatctatagatgttactctgggactctgacatacttccaggtgtgagttccatgaccatttgcataacttgatgtatccggattcatatacattttgataaaaaattaatttgttttaaaagatgagaggtagtgaggatgacatctctgtttagcagtgataagttaaagacctgttacctgcacagtaacccttcaagcccagaggctgaaactgatccctttagtatacgtagactgcttttattcccattcccatcctcaggttgtgatgtgcccgctgttgtgtcggcctttgtgttgtcagattatggcccaggctcctatttccaagcctgggcttcttctctttcccaagcaggtgaggcaggagggaggcagcgggcagagccttaggttgggtggggaagttcagatcttggccactggcttcttccctcttggattgctaatggctcaactctgtcatcctctctctccttttccagaaacccaaggccatcttgtgtccagccctctctgccctggttgtctacttgaaggctgtcacattctatagcttcactcactcgagggagcactaccacttctatgaaacatcatctttctctgaaaccaaggccaagagcctcatcaaggaggctggtcaggaccaagagggagggatggaaatgggatgggtaaagtcaggtctgacgggtaggaggatgagttccaagaaagaggatgagggagcagagggacgcggaagttaaggaactccccggaccaggtatggggataggaggtgaacagtggactcaaggctctagtggaaagacagctgggggaggtggtctgcatggagcagagacagtcacagccttaggagatgctggagagaagagtgaagtaggtgggcgggggctcttgggaccaaggaagtaggagggtccactccacccactaccatctcaggacccacaccctctttacaggtgacgagtttgtgcagcacaatgcctggcagttaagcagagtgtatcccagtggcctgaggacggattcatccaactacaacccccaggaattctggaatgcaggctgccagatgggtgagggagtggtagggacagggaagacggagtgcaagggcaatggatgggaagttggtccctgggtggcagcggggccagagagtgcttcaaggggagtgccgggctgagaaagacattctccagtgttgccagcagcccttttacacccaaaaatttctcagtctggtttccctggaagatggctgcctagtccccaaggtgactctttcctttttttctcctggggtcctcagtggctatgaacatgcagacagccgggctggagatggacctgtgtgatgggctcttccgccagaacgctggctgtggctatgtgctgaagccagacttcctgcgcgatgcccagagttccttccaccctgagaggcccatcagccctttcaaagcccagaccctcatcatccaggtataatggaaagaaaaaccactgggaagaaactgggatagctgaaatagagcctggggcaggggggaggggggcaggggcggctaggtctgacaggagtgtaatagagggaaagttcctcatggggcagcatggactttggagtcaatcgcctaggtccaaacccatttactagtcatgtaatgtttggcaaacttaaccttattctcctcacctgcaaaattggggtaatactgcctactgtacagaactgttgagattaatctgtgtgtgtgtatgtgtgtgtgtgctcagtcctgtctgactctttgtgaccccatggactgtggcccaccaggctcctctgtccatgtaatttcccaggcaagaatactggagtggttgccatttcctactccaggggatcttcccagggattgaacccgagtctcctgcactggcaggcagattcttgaccactgagccacctggggagcccctcattaatctgaagtgctgacaacagtgcctagggcacagaagtgctatagaagggttgctgtcataactttcccactttcaggccttattaaatttctacttggatgaattttaaattgcaggtgaggtactagaagaaaatatattacccatgagaattagttttgtaaaccataagcatatagtgcaaggtcctatgtctccccctcccctcagccttcctcctccccccagggtgtgagagctggaggggggctgggctgagcaggaactagtgaggttccagagccctggttgcaggtaatcagtggtcagcaactccccaaagtggacaacaccaaagaacagtccattgtggaccccttggtgagagtggagatctttggcgtccgcccagacacaacccggcaagagaccagctacgtggagaacaatggtgagacgctcggcagtgctggggaagtgggaggagcatggctgtggtttccccactgtccaggtctccgttaagcacagtccgtcacccttcctgcccctccaggggaagcagtgcagacaggacaaaggaggaggacttggtctgaagccaggtctctcacttacagtgacctaagcagactgcttatccactcggtttcctcacctgtaaagtgctgactgctctgaaaatgaagcaagagaacacagggcaagcaactcaccagcgccgtcaataatgtctgttccttcatcctttccctcactcaccctccctggctcaggttttaatccatactgggggcagacactgtgcttccggatcctggtacctgaactggccctgctgcgttttgtggtcaaagactatgactggaaatcccgaaacgacttcattggtcagtacaccctgccgtggagctgcatgcagcaaggtgagatggtcctccctctcccggcccacaccccagctctggccgtcttcctgatgctgtcctctccacccctccaaccacccccaggttaccgccacatacacctgctctccaaggatggcctcagccttcacccagcttccatctttgtgcacatctgcacacaggaagtgtcagaggaggctgaatcttaaggcagccacttcacaggcggagtggattcactggctgtagatggtgagaaaaatgaaaaaaacctggaaggatgctccagaaagcaaacagtggtaaaaacccgagctttggactgtgccttctcaggcacaaaatgacttggtccttcctgacaagcaaatgtaggacccgatttttcaccttctttctcttccctttcttcgttttcacaagcttctggcatcattcctgtcctttccctctgcagactctacactggaattccttccttcctctggctataggctcaacaccatacccagatctaggacagtgctttcccaccaactctggctcaaaggcacattgcagctagaaatccagagaagggcttgggggcagagaaacccaaaacctaaacctccacccctaacttcctcaaagtccagagccaagggaaagacaaatcaagcctcaagtctccccaggcaaaggctggggaaggagatctgatcccctgtattatgactctcaagagaacactgcatggctctgagagtccccactggactcctgctcacttgtgccaccagtataaatagagcttctctctcaaattggcatgagtgtggtctcttattgttgggcaaggagatatgagggttgggggacatttgggtaagacaggtggggtagaaaaaagtgggaaaacagggtttcccaggcccctcaggattcactacacagaaagagttaagaagtatgacttctacatggcagggtaaaaggagaagacagagagttacgtgatcccaggcttcctaaagagttggcctggaatagtcttgctttatgtctggtgtctcagcataattattaatagtaccctcttcgattttcccagtgtcccagtttagatgatttatgatacagtcactgtgaagcaccgacagaattctgggtgagagatttacctccccaactgtctgtcttccactatcctgtgcttcaactccagtccagcagctgagctggtctgaggatgctgctgaattaaagctgcttctatgagaaggctgaatgggtagcaatcctccatctactggaagacaaagggacttggaaataaggagaaggcaagggctaactcaggctctaggacagacagaaggcagacctggtcctgtttcagaaccaatcactcagtgaagatgaaggagacaaaggagtgaaggaggcctgagaggggacagggcgcactgagagacagcctggcagtattaaagtcggggacatggaagagtctatataagctacgaatcatttaaccatgctttatggagcagggggcacaatggcacatgcggttagatacaactgttttgaggatgctgaggaaataggaacttgacacagttaagaaggaaagagatgaagtgggtaatccacgatcttatagccagggactttggtggggcgatggtgaacaaattaagaacacacaggtcaaaaacacagtgtgcctcaacagaaatcaatttgtggtccaggtctctcttttatatcaacttccaagtactacgggccagattcccaggagggttccagcctcattt

SEQ ID NO: 2 - DNA, 24703 bp, Bos taurus
tctcaggagcccttgttaaaaatgaatattcctggacctcatccttagaaactctgatctggtagatttgaggtggggctaaggaatctctgttttaagacactctacaggagacattgatataaccaggaaccactagattgatgtgttgtatgagtcttcaagctctaacattctgaaggctatgacctgtgacgggagccatgaaccaggtttgtcataattctcctccaaagaaagagagtagacaggatgaatctcttccatcctgccctcagagagactgtgctgctgctaagtcgcttcagtcatgtctgactctgtgcgaccccatagacggcagccctcgaggctcccccgtccctgggattctccaggcaagaacactggagtgggttgccatttccttctccaatgcatgaaagtgaaaagtgaaagtgaagtcgctcagccatgtccgactcttctcgaccccatggactacagcccaccaggccctccatccatgggattttccaggcaagagtactggagtggggtgccattgccttctccgcagagagactgtagacacctcgaattccactataagccacaggcaactctaccaaagtccatctccaggtgatatgaagccacctgtcagctgaggaccaaattttgcttggcaacccttggccacttcctgccctatttgtgtgtctattttctcatctgtattgtagaggtaattatctgtatcctcagggagctccacatatgtgcctagacgtaaacagttaagacccaacctctctctcagtcattatcctctgattcttgcctctttctcttgctcccactcccaaatcctttttccctcctctccaaactgtacctgcctcagctgtgaccatcctccccattccaccttcgggtgccagaggaaacaccaagacctagaacacctaggttctaagttgcattctctgagtagtttgtttagtcagtctcctaggatacaggggaaaatgaagctgagcagaaggatctgttactcaggatcccaacccaagagtcccccttcctgaaccaaagtctcaagcacctttcaggggacaagaatccaagccattgcaaattagaagagaaacaaaagagcaaggagtaatgaggagaagggatgaggagaaggatgagaagtcaagagcaaagagaggctgacttaggtaagcaaataggaaggcaaagggagggtcaggagagagcaaagtgagctaggaagaaaggaaaaggaaggggtcaggtagaagagggggaaggggaagaaaataggaagaaggaaaagaaaggctgttgagtgggatatggtaggaacagaaaatgatggggtggcggggagggagaacaatgtccctcactgggtcccagctctgctttcatcggcttgctagagaaagccttttctaagggtagtctcggtgaggtctctgagttggtcaaatggagacctcagttctagccccagttctgccacttaattccttaggtgactggcagttcagctagccttctgtccctttattttttctgacaaaaggattcccatacttattactgagctccatagaccgctggtggggaaacagaaagcacggggagctaagcatttcgtctcccctgtgagttgttcaaagagcggtgatgttctgtgatctggttggttaacgtacaaccttaacacaggataaatgtgtgccacttcatacgtgaagcttctgacacagccagcttggttcttctccagtgtcttctcttggagttgtatctgattacctgttttcattggaatctattgaggaatgggatgattctgcttttttttgtggtcaggagttgcctgatcctgaaagtcttgtgagaagacatggtgagatgcaaactcaatcacacgtaggatggcagagaaaagagctcctctgtcccttttctacaaaatgaagtccagctctgacatcctgtggctccctgagttgaacctagccctctatcggccacgtgtcacctagatacagttttaaaacagtgactcaagaatgggagcagggcacgccaagctctgaggttgaaagagtttgacaaaagaatataaaatcattttctaaggctggtttgagatgaggtagggctgttcctgtgcttgggactgttctcagatctcccatgtctcgacattgtggtgaggctcccagacacaggcatatggaaggtaattctgtcttctcccagggccaaagcccacccaccccagaagttcagaggagagaggaggcaaaatgttggcaaaggggattagacttcagccccaggttgaccccaacccctcttagagtctgaaactctaagacatttaatgaagatctattactttttagtcttcatagaacttttgcaaaccttggattccaagctgtgcagcaccacccagcaaagcagagatgctttcctgtcttctttccttccttcttccatccagaggtctcctctggtctcactcaggattctacgtgcagcctcagccagtaaaacctttccctcttgggactgaagctgtgttcttgttctcctgagagttgccttatctcttctgcttagactcagattggtttcctgcacattcctactcctttggaaaatcttctctaggtccttcagtcaggagagctataatttcagtgacttctacatcattcctgaagatcaccaccctgggtggctttacaggctcttcctacttaagggagatggggcactaatatgctgtgtgacctcagacaagtcacatcatgtttctggatttctgctctaacgtagggagttgaactacatgatgtttaaatggactactaactgaataattagtttaaggattcttgccttttttttttttttagatgttcaccactgacaaattgttaaacttattcagggatgttcctggcagtccaggggttaagacttcacactttcactgtaggaggcacaggttcgatccctgattggggaattaagatcccacatgctgcaaagggcagctggaaagacttcttttaagtttaaacttactaattaaaacccacaatatttctcttcaaccatggatcctttgcttatacaagagcataaagctttatgttccgtggactttgccagtttcagcatgatttgttttaagcactgagtatatttgttctaggcacaaagctaggatcaaagtggccacaaatcaaatcatccagttcttttcaaggcaagtttgttgttagctcttaggataacttcagttcagttcagtcactcagtcgtgtccaactctttgcaaccccatgaatcacagcacgccaggcttccctgtccatcaccaactcccggggtttacccaaactcatgtccaccaagtcggtgatgccatccagccatctcatcctctgtcgtccccttctcctcttgccccccaatccctcccagcatcagagtcttttccaatgaatcaactcttcgcatgaggtggccaaagtattggagtttcagcttcagcatcagtgcttccaatgaacacccaggactgatctcctttaggatggactggttggatctccttgcagtccaaggaactctcaagagtcttctccaacaccacagttcaaaagcatcaattcttcggcattcagttttctttacagtccaactctcacatccatacatgaccaccaaaataaccagtcctaaatgagcaatggcaccccactccagtactcttgcctggaaaatcccatggatggaggagcctggtgggctgcagtccatggggtcactaagagtcggacacgactgagcgacttccctttcacttttcactttcatgcattggagaaggaaacggcaacccactccagtattcttgcctgagaatcccagggacgggggagcctggtgggctgccgtctatggagtcgcacagagtcggacacgactgaagtgactcagcaacagcagcagatgagcataaaaggtaggaactctctttcagtaggtagagaaaacttgaacatgctgttctgagactatgtaacttatctggttcattaaaactgtgagaattactattatgaataagagtatgagctatactttatcatgtgaaggtagaagtggctcatttagcaaggcgcagcactaagtgtggtagaagagacaaaagaaatactctggcctagcttgtccagagctggaggatccacacaagagcatggagagggctcctggaagggaagttgagatgattctgcttggagaaggaaaggctaggatgcagagagattccaagttctgaaagggggcagccagcaagtcagtgattcctaataatggggaactgagagaggtgtggtcaacatgacctcgtggattgtagggtgttggaaaagaagagggtatttggcagaaacccctgggttggccctgtcagagatggctgtgggaaacggcagaaactctgaaatcagatatatttgggtttaaattccaagtccctgtgtgattgagacaagtttcttagcctctctaagcttcaatcactaaactcaaattagaagtgaaaagagttacaaattatgtagatgaagtagctgacacaacgcaggcagtcaaggaacactaatttccttctcttcctccctcgatctattactctaagacctcaagattaacctgaagcctcttgctgctcctcgctcctttaggtgactctggcgtcagctggtgaagcagtgggtgatggcgtatctgctgcaaggccgtgagtacccctgcagaagagggcttggggagacagcccagagacagggaggaggctggactcctgttccaaagctcctcctgggccctccaccttccctaccggctactcagtccctgcttctcctggctcagggctgcccatcaatcaagatctgctgctgatgcagaaaggcacaatgatgcgcaaggtgaggtccaaaagctggaagaagctaagattcttcagacttcaggacgatggcatgacagtctggcatgcccggcaggctggaggcagggccaagcccagctgtgagtgatgtgggtagccaggggtgggcatgtgggtggacaggaccttacaggcccagcagcttgagggcagaggagtgaccagtgtcttatgatgtgccctgttctggtctctttagtgtctttatctgagcctgtaaaatggaaattataactgtactagcctcttcagggtgttacaaggatcaaattggttatggtgtgcaaagatcttccaagagtctttgccacataataaggctcaataaatcataggttttatcttattactatcatcatcatcatcattattatagcataatctcaggtagccaactgtctagacaaaagccctggctctgccaatttctagctgagtgactttggacaagttatttaacttaagctaatataagtttcctcatctataaaatgaggattaaacagcctagtacattacaggagctcaagaacttaatgtatataaaccacctagcatgaggcctgaaacccggatcaatttagtgagttaaccattatagtgaatatttctaaatatcgtaaatgcgtcataacagtgttcctcctattggccaaacactttatgatccctctaaaaatgtgaccacaggactcacctaagagaatctactttagagacataaccagagcttacgatccacaccagccccagctgaagaagcagggggaaggatttctctggctccctcccagacatctggaaagaggaagccctcaaacctggaatgagagaactgaatttgggcgcgtcatgctttggtgtaactgcttctatcctgcaagtgtgcttccacttgtcagccccaccagtcttcctgtaattgatcaccattggattggccatggaagggaccatcccttatttaagattaggatgctgaggggctacgtcattttcatccctgtcacactgccagttggtggctggaagccaggtcttgccatttccagcacaggccccttgtcatgctgctatgaaaacaacgactcatgtggcaatctgaattccaagaatctgcatctccctgtactaaggctttgggggttttagtctatggaggctggggtccctcctcctgtggtctcagagagaagaagggggagtactggtccttggctctcagctgctgtgatttgtcataccctttgcttagggccccccactaacttcttccctggcagtctcaatctccgacgtggacacagtacgtgagggccacgagtctgagttgctgcgcaacctggcagaggagttccccctggagcagggcttcaccatcgtcttccatggccgccgctccaacctggacctggtggccaacagtgtccaggaagcccagacctggatgcaggggctccagctgttggtgggctttgtcaccaacatggaccaacaggagaggctggatcagtatcagcaggatggagtcagagtgggatccaggggtcactgaaggagccagatgctgaagggctagggacaagtaggagggggcacagggctagaggggcctgagagtccagatggggtggccagggacagcttgggctgatgtgaggctgagatactgagggaagaatggaggcctgtgatggcacagacagagtcacacagagtttggagaggaatcagggcccccaggattgggtaaagggaccgtgagaggcaaactaggagctgatcctgagagctgaacagaagatcccgtggcattaactcccatagggatggggagcaactccttagggggcttagtgggatcaactcacttcaagtttcctagaatgtattcatcttctatgctagtccattcatttattcaccatctattaagcagaatgtaagaggcactatgccaggctttagggatacaaagattaatgaggctcagatattattttagtcagcagagaaaatagaattgtgtgaataatttaatttaccagcattacagtacttagctgatatgtgggtgttcaaaggaaggagaaatacttatttctagctgagatggtcatgagaaagcttcaggagaaagtagcgtttgagctgagccttgaaaatgagtagaatttgaactgaagggaagaggaggggaaggcacttgtgtctgaggaaacatcatgaataaagacccagaggtaggaagctgaagagctgggacatcaccaagcaggggcttgctgaatctgtctgaggtacaaggtagtcatttctgtgttgagaaggctggtttggggccagatcagtggggtgaaggagatcttaatttctatgttagggaagttgggcttctttttgcaggcaacagggatgtccccaaaggcacctgagccatgcaggggcaagatagattggtgctctggggagattattccagaagctaatatggaaaatgaattggctctggggaggggactggcaacaaagacaggggagcagtcaatggatgaatgtgcacacacatctgcacacacaaacacacactcagtgcttttgccttgactcagacctggatggctgagtgattggttccagcgcggagacaaaaaccaggatggtcggatgagtttcggagaggtccaacggttattgcacctgatgaatgtggaaatggaccaggaatatgcctttcagcttttccaggtgagtcttctggggaaaggttagcagaagccaatcaaggccacattctcccttagccagaagtcctccaaccctctccagcaacacacagctgttgaatctctcctactacgccaggttcattgggaagtgtgggtaggacatggttcttacccttcagtcttgttggaaagatgactcataatcttatgaaaggtaaataacaacatgttattacatcagacctcagggacctctctgtccttggccaggatttcacaagtgcctagtacagtggaaaactccaactggcatgtgagtaaatagaaaaagggatcttctgatcttttagggacatgttgggggcatttaattctttctagttgaaatttttctccagtagcaaatggtatggccgaaattttgctttttacttcctgtcgtggcttttcctatgaacactaaatgtcaggagttctgtggaagccagcctggctccatcaataataacgcccttacaaggaaagtcaaaactgcaacatctgccgttaaagggttttttactctcctctaaaccctcttctgcagacagcagacacatcccagtctgggactctggagggagaagaatttgtagagttctataagtcattgactcagcgtccagaagtgcaggaactgtttgaaaagttttcatctgatgggcagaaactgaccctgttggaatttgtggatttcctccaagaggaacagaaggaaggagaacgggcttctgaccttgctctggaactcatcgaccgctatgagccttcagaaagtggtaagaggactagggtggtgaggcaacacacatgggggccagatgggggaccaagagacaactagccttctgcctaactaagggaaaattcagtcccccagaccccgttcccaatcatcttcatctcattcaatcactcctccagaaggagaaagccatttcctgcccacataagacaggagtctcctctgtagtgctgtatctcttaggaggaagaacattccgtaagtcccctttcctctaccacgctcatatttctcattcttcactttcacactgttcttcctgatatcaacctttgtcctcagtggaaacataaaatccttctcctcttacttttggcttctctagggcatgtgattcctttccctttagttcccttcatggttcctgtttgctgtctctttaagtgctggctctctgccctttcctggactttctccaagttccccaggtggtttttgagtcctaagtccctgagctgggcacctagtcagcccctggctgaggtggatgatgaggtagacaatcatttggctcttaacaagcagttaaaatatgccctctctgccctcaaaccttctccttgcccctttagtagataccttcctgcccttcattccttgcagagcatgaaaaacttacattattaataaaaagagggcttccctgatagctcagttggtaaagaatccacctgcaatgcaggagaccccagtttgattcctgggttgggaagatccgccggagaaaggataggctatccagtccaatattcttgggcttcccttgtggctcagctggtaaagaatcctcctgcaatgcgggagacttaggttcaatccctgggttgggaagatcccctggataaggcaaaggctacccactccagtattctggcctggagaattatatggactatatatatagtccatggggtcgcaaagagtctgacacaactgagtgacttccactttacttcactttatagctcttacattatttagtcctttgtgtgtgtgtgtgtgagtgtgtgagtgtgttagacgctcaatagtgtccaactctttgcgaacccatggactatagcccaccaggctcctctgtccatgggatttcccaggcaagaatacaggagtgggtaggcattcccttctccaagggctcttcccaactcagggataaaaccccaggtctcctgcactgcaggcagattctttaccatctgagccaccagggaagcccttagtactcactgctgctactgctgctgctaagtcgcttcagtcgtgtccgactctgtgagaccccatagacggcagcccaccaggctccgccatccctgggattctccagacaagaacaccggagtgggtttagtactcactacatccttgtaaatcaaaagcaataatctttattgggcctttgctaagcatcccataatgtgctgatatctctacatgccttttctcttctaatccttccaacaacccatggaagtagatttctattatcaactcagttgtacagaagaggaaaactgagattcagaaaaattaaatgacttgctcaaagtcaacagctaatgaaaggaggaattgtaattcaaagccagatcattgactcttacctggtgcctctcttgagaatgagaataagacatgagaggaatgtgagctggacaggtgacccaggtttcttaggtctcaattcccattccctttcattcactcattcattcattcactcactacttcagcaaatgctcactgagtacctataatgtgccaggaccaatgataggcaacagagatacagtagtaggtcagacaaggacactaccctcatggaccgtggagccaaacaaagtgttgcaatcactgattccagatttatgtgggtgcttagagaagaaaggggctgtgacgcaatcttttcagaaaactaaacagaagaggaagttgaagggttaagacaggatcctctgtttccaatgaaaaatccagcatacatcattacatttattctgcctgtgtgcatgctcagtcactcagtcgtgtccaactctttgtagcccatggactgtagcccaccaggctcctttgtccatggaattttccaggcaagaatactggagtaggttgtcatttcttactccaggggaccttcccaacccagggactggatccttgtctcttgggtttcctgcactggcaggcagattctttaccattgagccacctgggaagccccacatttattctatccaccctttttggagttctttcccttttcacttgattatcagatacaacaacactgtggctgcaaagtgtggtatcagtcaggagacagaggctcaagtccccactctgcttcttatagagtactgagttgcttaacctatcggaggctcagttttctcatttgtaaaataactggtgatcaaatgaaataaagcacatgaaaagtacctggtagctatggactttatcaatttgcaatttgagtcattacagtcaagaatgaaactgtgggatttctctggcagtccagtggttaagactttgaacttacactgaagggggcatggatttgatctctgatcgggaaactagcaccctgcacgcctcaagtctggagaaggcaatggcaccccactccagtactcttgcttggaaaatcccatggacggaggagcctggtaggctgtagcccatggggtcactaagagtcagacacgactgagcgacttcactttcacttttcactttcttgcattggagaaggaaatggcaacccactccagtgttcttgcctggagaatcccagggacgggggagcctggtgggctgccgtctatggggtcacacagagtcggacacgactgaagcgacttagcagcagcagcagcatgcctcaagtcatggccaaaatatagaaagaatgaaactttgcgcccaacttcttgccattttccaccatcgccaggccttgtcacacctggaatgaggaagctaggcatgagagtaaagctgttctgcctcccctgtagcaaagttatcctcagtgagacttcacagtccagcagtgtagggcagattctgacatctgactccctgggttcaaatcccagctccttcacttattagttatgtgacataatattatataggaggcggtgaccagaaccatcccaaagaaaaagaagtgcaagaaggcaaagttgttgtctgaggaggctttacaaatagctgagaaaagaagagaagcaaaaggcaagggagaaagggaaagatacccaactgaatgcagagtttcagagaatagaaagaagagatatgaaaaccttaagagaacagtgcaaagaagtagaggaaaacaacagaaaaagaaagactagagacctctttaagaaaagtggagatatcaagggaacatttcatgcaagaatgggcacaataaaggacagaaacagtgaagacctaacagaagcagaggagattaagaagaggtggcaagaatacacacaagaactgtacaaaaggtcttagtgatctcgatttccacgatggtgtggtcgctcacctagaacaagacatcctggagagtgaagtcaggtggaccttaggaagcattactatgaataaagatagtggaggtgatggtattccagctgagctatttaaaatcccaaaagatgatgctgttaaagtgctgcacccaatatatcagcaaatttggaaaactcagcagtggccataggactggaaaaggtcagttttcattccaatcccaaagaagggcaaagccaaagaatgttcaaactaccacacaattgcactcatttcacatgttagcaagtttttaatgctcaaaatccttcaaactaggcttcagtagtacacaacagatatacaagctgggtttaaaaaaggcagaggaaccagagatcaaattgttaacattctttggatcatagaaaaagcagtaaaggaattccagaaaaacatctacttctgtttcattgattatgccaaagtctttgactgtgtggatcacaataaactgtggaaaattcttcaagagatgggaataccagaccaccttacctgtctcctaagaaacctgtatgcaggacaaggagcaacagttagaactgtacatggaacaaggactgcttcagaattgggaaaggagtatgtcaaggctgtgtgttgtcatcctgcttatttaccttatatgcagagtatcttatgtgaaatgcgaggctggatgaatcacaagctggaatcaagattgccaggagaaatattagcaacctcaaatatacaaatgataccactctaatggcagaaagtgatgaggaacaaaacagcctcttggtgagggtgaaagaggagagtgaaaaagctggcttaaaactcaacattcaaaaaacgaagatcatggcatccagtcccatcactttgtgcaaatagagggggaaaagtggaaacaacgacaaattttatttccttgggctccaaaatcactgcagacagtgactgtgtgcgtgctaaatcacttcggtcatgtacgattctgtgcaaccctagggacggtagcctgccaggctcctctgtgcatggaactctccaggcaagaatactggagagggttgccatttccttctccaggggatcatcttgactcagggatcaaaccagggtctcttatgtctcctacagtggcaggcaggatctttaccactagccacctgacagtgactgcagccatgaaattaaaagacttgctccttggaaggaaagctgtgacaaacctagacagcatattaaagaacagagacatcactttgctgacaaaggtccgtatagtcaaacctatggtttgtccagtagtcctgtatggctgtgagagttggaccataaagaaggccgaatgcctagaaattgatgctttcgagttgtagtgctggagaagcctcttgagagtcctttggacagcaagaagatctaaccagtcaatcctaaaggaaatcaaccctgaatatttactgaaaggactaatgctgaagtttcaatactttgaccacctgacatgaaaagccgactcattagaaaagaccctggtgctgggaaagattaaaggcaggaggagaaggggatgacagaggttgagatggttggatggcatcactgactcaatggacatgagttagagcaaactctggcggacagtgaaggacagggaagcctggcatgccctagggctgcaaagagtcagacatgactgagcgactgaacaacaaccacaaatgtgacccagggtatgttacctaacctctcagggcagattatgttatatccacctgattgtgttggtgagaggattaagggagatgctgcatgttatgtccttggcaacgctcaagaaaccctcaggccatagtaggcagtagctacacagcccagggcataacacacaggtaccttggtcttggggcttcccaggtggtgttagagctgaagaacgcctgtgccaacataggaaatgtaagacatgtaggttcgatccctgggttgggaagatctcctggaggaggcatggcaaccctctccagtattcttgcctgaagaatctcgaggacagaggagcctggcaggctacagcccatagggtaacaaagagtcttcagacgtaaagcaacttggcattcatgcatgcaccttggtcttgtcactaacaagtctttgactttgatcctctctgagcctcgggtgccccatctatgaaattagtcctttggactaaatactgtgctgtccttcatggtaaccctgagccccatgtgactatttaaatttaaattacagtcggctctctgtatctacaggtgccacatcgcggatacagagggtcgactgtaccatgagattctgtgtaaggcacttgagcgtccatggattttggtatctgctatggggaaggggccggagaaggcaatggcaccccactccagtactcttgcctggagaatcccagggacaggggagcctggtaggctgcagtccatggggttgctaagagtcagacatgactgaatgacttcactttaactttttactttcatgcattggagaaggaaatggcaacccactcccgtgttcttgcctggagaatcccagggacaggggagcctggtgggctgccgtccatggggtcgcacagagtcggacatgactgcagtgacttagcagcagcagcatggggaagggggaagtcctggagccaatctcctgtgaatacaaaacagtaattcagttccacagtcacacagcaacatttcaaatgctcagtagctttatttatttagaggttatcatactgggctgtgcagaccagagaacacttccatcactgtagcaagttctgttgggcagcactggactagcatgatcttaataattccctttgtatttttatggttctctgaggatggggatgcttgaaatgacaggctggatagggcagtggggtctggaggctgggagtggacaccatttgaaaggctgggccctggagaggggtggggtgctctgagcagagagcttctcactctagccactccacaccccaggaaaactgcggcatgtgctgagcatggatggcttcctcggctacctctgctcgaaggacggagacatcttcaatccaacctgccaccccctctaccaggacatgactcaacccctgaaccactactacatcaactcctcccacaacacctacctggtgggggaccagctttgtggccagagcagcgtggagggatacatacggtgctgtggggctggggggtgaaggggtccagctcatgagatgggaaggtctgtgggaagttggaggggaattgttagttgactgcaggaattgaaatgctgttgtggggggatttgagctgaggattccttggtccctgagagacttggaggagatattgaagactggcaggagagtgctgtggaaaccctgcgggtaaacacagcttctctttcataggacatgaaattaccttggcaccagaggtcctgacaaatttattttgaacaaataaataaactcaatgttaaatgggaggcagtagggaagaggatttaaaaacatatgttctggagtcagaccatttaagatcaaccactagctccctcacttggtactctgaggtcaaatacttaacctctctctgcctcagcttccctatctgagacacagagaggattactattctgtctattctgtctagcttgtcgtgaggattcaataaggtaatttatgtgtcaataggtaataaatggtacctgactttattgtgattaaccatgcaataggtcaaatagtcaaggcatctttgtaatgtcagcagctgaactagtcgcttcaagtgcaacttccctaggaagcctagcaaggaagtaaaatgtaaatttactgagagtttgaataactcaggtgtcatataaagatatacaagcagaacaccagaaggcagtatgtgatggctccctaagggaggattgttgagtagggcctggtggagatcagtgtgagctgcaggtgtgtcccggcagacagacaggggtccacggagatgtaaattgacagtcttgatgtgtgagtgccagtgagcactgttctctccagagagggaagagggtccctatgtgaaagatcacaccttgacccctcccgtccctggggggtggggttcttgcaggaccctgaagcgggggtgccgctgtgtggaggtggatatatgggatggaccaagcggggaacctattgtttaccacggacacaccctgacctcccgcatcccattcaaagatgttgtggccgctataggacagtatgccttccaggtgggagcctcaggatggaaatactggtgattagcaagaggatctgaaggaagagactctgggtctggggttggggagggatgcaggggaaagtaaaagtattagtcactcagttgtatccaactctttgtgaccccatggactgtagcccaataggctcctttatccatagaattctccaggcaagaacactggagtgggtatccattcccttcgccaggggatcttcccgacctaggtgatgtcaaatgacatatacgtaaaagagattttaattgtgaagcatcaggcaaacattaagggattattatgaataagtgttggatgtgttactagttaagtagtcatagaagtaattgctaactgtaatgattacttctaaaaacttctgtcacccacctgggagagggcggaggaggaaacagacaaactggtgggtagagctgggaggcaatagctatctggggatgaaagtctgaaagtataacgtgaacatgtctggggcactggcctggctttcccactctaagcctgaaaactcaaaggtcccataaggacctgggtcagacctggcacagatctgagatttggggtcccaagaatgacttttagatgtggggtgcctagacccaggcagatctcctggaggcctggcatggggtcagaggacttttggcaatcagggtactcagttgagtaattggcagggggtgggtggtggggtggtgggtgggggtggagctggagaatcaagaaggttataggcctcagaactctggctccagcatgccttcccctcctgaccagacgtcagactatccagtcatcttgtcgctggagaaccactgcagctgggagcagcaggagatcatcgtgcgccatctgacggagatcctgggggatcagctactgacgacagccttggatgggcagcctcccactcagctgccctcccccgaggtagggaccctgccttctagcccaggcttccctaccgggcttggctcttcctcccaggcccatctgttcccttcttctcaccctcagatggaccgtcttgctctttaatattctcagagacaactttaatttttaaaaaatcgaattcagggtgttccagtgtggcctagtggttatgattccaagctttcactgccaatccctgcttcaggaactgagatcctgcaagccgagtggcatggccaaaagaaatttaaaaaaatctaattcaacataaaactccagatatagctttctaaatttaaaaccatttgtttactttatatttcttttttcaatggaagatcttcattttttttcctgatagttcattattataggctctacaaatataaggggagattcccccaccataatctcaacctctccaataacagctgccaacattatagattttttatacacttatatagacatatatatgtatatatggttacatttttaaaaaatgtgaatgggataatcctttctatcccttttatatatatcacaaatattttttcatatatattttcttttttaacagttctatagtattaaactttatgttactgaccataatttactcaaccagtaggtggtaaaaataaccagtaaatggtaaacatttaggttgtttataatttttcatttgtacatagagctttgctcatttactctttctttgaaatggattcctaggaaaaaaattgctgggttgaaaactgcatgttgacatttttggttattattgtcaagttgccctcgtgggggggggcagggtgcagaaaaaaaccaaatttcactccaactgaacagtgtaaggggatgactacaaacaactccagtagtgagatgattcgtaatggcaaacatgaaaaaaaagtttttaaaaaacataggtctgttttattctttcaaattaactgatattatttacaaacttttttatattagagtttataatttttcttgatatataactttaaaaaatgttaaagatattaatgctttaaaaatacttttttgcagtctatttgggcaggggtggccacgctgtgcgacttgcatgacccagttcaaaaccagggactgaacccaggacacagccgtgtggagtcctaaccactggaccaccaggaaactccccttgcagtctattttttctttttgtcttcatagcctcttctgttttacaggtttttaacctttatgtagtcacctctgccaatcattttttatgtcttctgagttttcaaaggctttcctaccttgaggcttttcagaaagagtctctatcttcctctaatactctaatgttctttacatgtaaatcttgaatctatgaggaatctttacgtctcaaatgtgaggtaggggttcgcatgtattttttaccttagagatgttacaggtcggcttaaccataagaaatccctgctctatgtatcaagtagccttcctgtgggaacttccgaccacttacttgcttcctttgcctttgagatggtcctgtgagagctgcataggagctgcacgacccacaggaggggcccagagcagacaggtagacaggcccctctgtttttgtcccctaggaccttcgggggaagatccttgtgaaggggaagaagctaatgcttgaggaagaggaggaagaaccagaggctgaattggaagcagagcaggaggctaggctggacttggaggcccagctggagtctgagccccaggacctgagccctcgcagtgaggacaaaaagaaggtaggccagaagggtggtctttctgctctggcgtttgattagtcagtactacaagctgagaccccatgtgtgaagagactggagccaggcagggtggagaaccaaggagcaaaatgctccttgcctccaatgacaagaggcatgagaatccatacacagcatcatcatatagattgccatatacagacatatggcagtggctttccaacttctttgttctgtgagatctttttttttttttttaatgaaattctatatggaagctcaggtcacaaaatagatatatgcagagtcattctggttgaagtacttctttggctctctcctgggtctcccaggtgacttctgagacatctttgtactacttcccagtctctttaaatgatacttcaaaaaccactgatgtgtgcatgtgtgttgttatattaggtaagtagggcttactagagtgactgctatgacctcaactaaagtgggtttattgctagaaagtttcttggaaaaggtgagtctgaaaagggaccagaaatagccaaagaggaggagcatgaaaggttggggaagggctgccaacagtgggatcaccaagatcttgctttgaagtctgcatttctctctcttatgcacttgcttggctcagctgaatgacttctagctaattatcctgttctctctggagctgtgttgtctcacctgctgccatgcccagggatggaaatgggaattcagaaaaatctatagatgttactctgggactctgacatacttccaggtgtgagttccatgaccatttgcataacttgatgtatccggattcatatacattttgataaaaaattaatttgttttaaaagatgagaggtagtgaggatgacatctctgtttagcagtgataagttaaagacctgttacctgcacagtaacccttcaagcccagaggctgaaactgatccctttagtatacgtagactgcttttattcccattcccatcctcaggttgtgatgtgcccgctgttgtgtcggcctttgtgttgtcagattatggcccaggctcctatttccaagcctgggcttcttctctttcccaagcaggtgaggcaggagggaggcagcgggcagagccttaggttgggtggggaagttcagatcttggccactggcttcttccctcttggattgctaatggctcaactctgtcatcctctctctccttttccagaaacccaaggccatcttgtgtccagccctctctgccctggttgtctacttgaaggctgtcacattctatagcttcactcactcgagggagcactaccacttctatgaaacatcatctttctctgaaaccaaggccaagagcctcatcaaggaggctggtcaggaccaagagggagggatggaaatgggatgggtaaagtcaggtctgacgggtaggaggatgagttccaagaaagaggatgagggagcagagggacgcggaagttaaggaactccccggaccaggtatggggataggaggtgaacagtggactcaaggctctagtggaaagacagctgggggaggtggtctgcatggagcagagacagtcacagccttaggagatgctggagagaagagtgaagtaggtgggcgggggctcttgggaccaaggaagtaggagggtccactccacccactaccatctcaggacccacaccctctttacaggtgacgagtttgtgcagcacaatgcctggcagttaagcagagtgtatcccagtggcctgaggacggattcatccaactacaacccccaggaattctggaatgcaggctgccagatgggtgagggagtggtagggacagggaagacggagtgcaagggcaatggatgggaagttggtccctgggtggcagcggggccagagagtgcttcaaggggagtgccgggctgagaaagacattctccagtgttgccagcagcccttttacacccaaaaatttctcagtctggtttccctggaagatggctgcctagtccccaaggtgactctttcctttttttctcctggggtcctcagtggctatgaacatgcagacagccgggctggagatggacctgtgtgatgggctcttccgccagaacgctggctgtggctatgtgctgaagccagacttcctgcgcgatgcccagagttccttccaccctgagaggcccatcagccctttcaaagcccagaccctcatcatccaggtataatggaaagaaaaaccactgggaagaaactgggatagctgaaatagagcctggggcaggggggaggggggcaggggcggctaggtctgacaggagtgtaatagagggaaagttcctcatggggcagcatggactttggagtcaatcgcctaggtccaaacccatttactagtcatgtaatgtttggcaaacttaaccttattctcctcacctgcaaaattggggtaatactgcctactgtacagaactgttgagattaatctgtgtgtgtgtatgtgtgtgtgtgctcagtcctgtctgactctttgtgaccccatggactgtggcccaccaggctcctctgtccatgtaatttcccaggcaagaatactggagtggttgccatttcctactccaggggatcttcccagggattgaacccgagtctcctgcactggcaggcagattcttgaccactgagccacctggggagcccctcattaatctgaagtgctgacaacagtgcctagggcacagaagtgctatagaagggttgctgtcataactttcccactttcaggccttattaaatttctacttggatgaattttaaattgcaggtgaggtactagaagaaaatatattacccatgagaattagttttgtaaaccataagcatatagtgcaaggtcctatgtctccccctcccctcagccttcctcctccccccagggtgtgagagctggaggggggctgggctgagcaggaactagtgaggttccagagccctggttgcaggtaatcagtggtcagcaactccccaaagtggacaacaccaaagaacagtccattgtggaccccttggtgagagtggagatctttggcgtccgcccagacacaacccggcaagagaccagctacgtggagaacaatggtgagacgctcggcagtgctggggaagtgggaggagcatggctgtggtttccccactgtccaggtctccgttaagcacagtccgtcacccttcctgcccctccaggggaagcagtgcagacaggacaaaggaggaggacttggtctgaagccaggtctctcacttacagtgacctaagcagactgcttatccactcggtttcctcacctgtaaagtgctgactgctctgaaaatgaagcaagagaacacagggcaagcaactcaccagcgccgtcaataatgtctgttccttcatcctttccctcactcaccctccctggctcaggttttaatccatactgggggcagacactgtgcttccggatcctggtacctgaactggccctgctgcgttttgtggtcaaagactatgactggaaatcccgaaacgacttcattggtcagtacaccctgccgtggagctgcatgcagcaaggtgagatggtcctccctctcccggcccacaccccagctctggccgtcttcctgatgctgtcctctccacccctccaaccacccccaggttaccgccacatacacctgctctccaaggatggcctcagccttcacccagcttccatctttgtgcacatctgcacacaggaagtgtcagaggaggctgaatcttaaggcagccacttcacaggcggagtggattcactggctgtagatggtgagaaaaatgaaaaaaacctggaaggatgctccagaaagcaaacagtggtaaaaacccgagctttggactgtgccttctcaggcacaaaatgacttggtccttcctgacaagcaaatgtaggacccgatttttcaccttctttctcttccctttcttcgttttcacaagcttctggcatcattcctgtcctttccctctgcagactctacactggaattccttccttcctctggctataggctcaacaccatacccagatctaggacagtgctttcccaccaactctggctcaaaggcacattgcagctagaaatccagagaagggcttgggggcagagaaacccaaaacctaaacctccacccctaacttcctcaaagtccagagccaagggaaagacaaatcaagcctcaagtctccccaggcaaaggctggggaaggagatctgatcccctgtattatgactctcaagagaacactgcatggctctgagagtccccactggactcctgctcacttgtgccaccagtataaatagagcttctctctcaaattggcatgagtgtggtctcttattgttgggcaaggagatatgagggttgggggacatttgggtaagacaggtggggtagaaaaaagtgggaaaacagggtttcccaggcccctcaggattcactacacagaaagagttaagaagtatgacttctacatggcagggtaaaaggagaagacagagagttacgtgatcccaggcttcctaaagagttggcctggaatagtcttgctttatgtctggtgtctcagcataattattaatagtaccctcttcgattttcccagtgtcccagtttagatgatttatgatacagtcactgtgaagcaccgacagaattctgggtgagagatttacctccccaactgtctgtcttccactatcctgtgcttcaactccagtccagcagctgagctggtctgaggatgctgctgaattaaagctgcttctatgagaaggctgaatgggtagcaatcctccatctactggaagacaaagggacttggaaataaggagaaggcaagggctaactcaggctctaggacagacagaaggcagacctggtcctgtttcagaaccaatcactcagtgaagatgaaggagacaaaggagtgaaggaggcctgagaggggacagggcgcactgagagacagcctggcagtattaaagtcggggacatggaagagtctatataagctacgaatcatttaaccatgctttatggagcagggggcacaatggcacatgcggttagatacaactgttttgaggatgctgaggaaataggaacttgacacagttaagaaggaaagagatgaagtgggtaatccacgatcttatagccagggactttggtggggcgatggtgaacaaattaagaacacacaggtcaaaaacacagtgtgcctcaacagaaatcaatttgtggtccaggtctctcttttatatcaacttccaagtactacgggccagattcccaggagggttccagcctcattt

SEQ ID NO: 3 - protein, 791 aa, Bos taurus
MAYLLQGRLPINQDLLLMQKGTMMRKVRSKSWKKLRFFRLQDDGMTVWHARQAGGRAKPSFSISDVDTVREDHESELLRNLAEEFPLEQGFTIVFHGRRSNLDLVANSVQEAQTWMQGLQLLVGFVTNMDQQERLDQWLSDWFQRGDKNQDGRMSFGEVQRLLHLMNVEMDQEYAFQLFQTADTSQSGTLEGEEFVEFYKSLTQRPEVQELFEKFSSDGQKLTLLEFVDFLQEEQKEGERASDLALELIDRYEPSESGKLRHVLSMDGFLGYLCSKDGDIFNPTCHPLYQDMTQPLNHYYINSSHNTYLVGDQLCGQSSVEGYIRALKRGCRCVEVDIWDGPSGEPIVYHGHTLTSRIPFKDVVAAIGQYAFQTSDYPVILSLENHCSWEQQEIIVRHLTEILGDQLLTTALDGQPPTQLPSPEDLRGKILVKGKKLMLEEEEEEPEAELEAEQEARLDLEAQLESEPQDLSPRSEDKKKVVMCPLLCRPLCCQIMAQAPISKPGLLLFPKQKPKAILCPALSALVVYLKAVTFYSFTHSREHYHFYETSSFSETKAKSLIKEAGDEFVQHNAWQLSRVYPSGLRTDSSNYNPQEFWNAGCQMVAMNMQTAGLEMDLCDGLFRQNAGCGYVLKPDFLRDAQSSFHPERPISPFKAQTLIIQVISGQQLPKVDNTKEQSIVDPLVRVEIFGVRPDTTRQETSYVENNGFNPYWGQTLCFRILVPELALLRFVVKDYDWKSRNDFIGQYTLPWSCMQQGYRHIHLLSKDGLSLHPASIFVHICTQEVSEEAES

SEQ ID NO: 4 - protein, 791 aa, Bos taurus
MAYLLQGRLPINQDLLLMQKGTMMRKVRSKSWKKLRFFRLQDDGMTVWHARQAGGRAKPSFSISDVDTVREDHESELLRNLAEEFPLEQGFTIVFHGRRSNLDLVANSVQEAQTWMQGLQLLVGFVTNMDQQERLDQWLSDWFQRGDKNQDGRMSFGEVQRLLHLMNVEMDQEYAFQLFQTADTSQSGTLEGEEFVEFYKSLTQRPEVQELFEKFSSDGQKLTLLEFVDFLQEEQKEGERASDLALELIDRYEPSESGKLRHVLSMDGFLGYLCSKDGDIFNPTCHPLYQDMTQPLNHYYINSSHNTYLVGDQLCGQSSVEGYIRTLKRGCRCVEVDIWDGPSGEPIVYHGHTLTSRIPFKDVVAAIGQYAFQTSDYPVILSLENHCSWEQQEIIVRHLTEILGDQLLTTALDGQPPTQLPSPEDLRGKILVKGKKLMLEEEEEEPEAELEAEQEARLDLEAQLESEPQDLSPRSEDKKKVVMCPLLCRPLCCQIMAQAPISKPGLLLFPKQKPKAILCPALSALVVYLKAVTFYSFTHSREHYHFYETSSFSETKAKSLIKEAGDEFVQHNAWQLSRVYPSGLRTDSSNYNPQEFWNAGCQMVAMNMQTAGLEMDLCDGLFRQNAGCGYVLKPDFLRDAQSSFHPERPISPFKAQTLIIQVISGQQLPKVDNTKEQSIVDPLVRVEIFGVRPDTTRQETSYVENNGFNPYWGQTLCFRILVPELALLRFVVKDYDWKSRNDFIGQYTLPWSCMQQGYRHIHLLSKDGLSLHPASIFVHICTQEVSEEAES

SEQ ID NO: 5 - DNA, 3228 bp, Bos taurus
acaggggaaaatgaagctgagcagaaggatctgttactcaggatcccaacccaagagtcccccttcctgaaccaaagtctcaagcacctttcaggggacaagaatccaagccattgcaaattagaagagaaacaaaagagcaaggagtaatgaggagaagggatgaggagaaggatgagaagtcaagagcaaagagaggctgacttaggtgactctggcgacagctggtgaagcagtgggtgatggcgtatctgctgcaaggccggctgcccatcaatcaagatctgctgctgatgcagaaaggcacaatgatgcgcaaggtgaggtccaaaagctggaagaagctaagattcttcagacttcaggacgatggcatgacagtctggcatgcccggcaggctggaggcagggccaagcccagcttctcaatctccgacgtggacacagtacgtgaggaccacgagtctgagttgctgcgcaacctggcagaggagttccccctggagcagggcttcaccatcgtcttccatggccgccgctccaacctggacctggtggccaacagtgtccaggaagcccagacctggatgcaggggctccagctgttggtgggctttgtcaccaacatggaccaacaggagaggctggatcaatggctgagtgattggttccagcgcggagacaaaaaccaggatggtcggatgagtttcggagaggtccaacggttattgcacctgatgaatgtggaaatggaccaggaatatgcctttcagcttttccagacagcagacacatcccagtctgggactctggagggagaagaatttgtagagttctataagtcattgactcagcgtccagaagtgcaggaactgtttgaaaagttttcatctgatgggcagaaactgaccctgttggaatttgtggatttcctccaagaggaacagaaggaaggagaacgggcttctgaccttgctctggaactcatcgaccgctatgagccttcagaaagtggaaaactgcggcatgtgctgagcatggatggcttcctcggctacctctgctcgaaggacggagacatcttcaatccaacctgccaccccctctaccaggacatgactcaacccctgaaccactactacatcaactcctcccacaacacctacctggtgggggaccagctttgtggccagagcagcgtggagggatacatacgggccctgaagcgggggtgccgctgtgtggaggtggatatatgggatggaccaagcggggaacctattgtttaccacggacacaccctgacctcccgcatcccattcaaagatgttgtggccgctataggacagtatgccttccagacgtcagactatccagtcatcttgtcgctggagaaccactgcagctgggagcagcaggagatcatcgtgcgccatctgacggagatcctgggggatcagctactgacgacagccttggatgggcagcctcccactcagctgccctcccccgaggaccttcgggggaagatccttgtgaaggggaagaagctaatgcttgaggaagaggaggaagaaccagaggctgaattggaagcagagcaggaggctaggctggacttggaggcccagctggagtctgagccccaggacctgagccctcgcagtgaggacaaaaagaaggttgtgatgtgcccgctgttgtgtcggcctttgtgttgtcagattatggcccaggctcctatttccaagcctgggcttcttctctttcccaagcagaaacccaaggccatcttgtgtccagccctctctgccctggttgtctacttgaaggctgtcacattctatagcttcactcactcgagggagcactaccacttctatgaaacatcatctttctctgaaaccaaggccaagagcctcatcaaggaggctggtgacgagtttgtgcagcacaatgcctggcagttaagcagagtgtatcccagtggcctgaggacggattcatccaactacaacccccaggaattctggaatgcaggctgccagatggtggctatgaacatgcagacagccgggctggagatggacctgtgtgatgggctcttccgccagaacgctggctgtggctatgtgctgaagccagacttcctgcgcgatgcccagagttccttccaccctgagaggcccatcagccctttcaaagcccagaccctcatcatccaggtaatcagtggtcagcaactccccaaagtggacaacaccaaagaacagtccattgtggaccccttggtgagagtggagatctttggcgtccgcccagacacaacccggcaagagaccagctacgtggagaacaatggttttaatccatactgggggcagacactgtgcttccggatcctggtacctgaactggccctgctgcgttttgtggtcaaagactatgactggaaatcccgaaacgacttcattggtcagtacaccctgccgtggagctgcatgcagcaaggttaccgccacatacacctgctctccaaggatggcctcagccttcacccagcttccatctttgtgcacatctgcacacaggaagtgtcagaggaggctgaatcttaaggcagccacttcacaggcggagtggattcactggctgtagatggtgagaaaaatgaaaaaaacctggaaggatgctccagaaagcaaacagtggtaaaaacccgagctttggactgtgccttctcaggcacaaaatgacttggtccttcctgacaagcaaatgtaggacccgatttttcaccttctttctcttcccattcttcgttttcacaagcttctggcatcattcctgtcctttccctctgcagactctacactggaattccttccttcctctggctataggctcaacaccatacccagatctaggacagtgctttcccaccaactctggctcaaaggcacattgcagctagaaatccagagaagggcttgggggcagagaaacccaaaacctaaacctccacccctaacttcctcaaagtccagagccaagggaaagacaaatcaagcctcaagtctccccaggcaaaggctggggaaggagatctgatcccctgtattatgactctcaagagaacactgcatggctctgagagtccccactggactcctgctcacttgtgccaccagtataaatagagcttctctctcaaaaaaaaaaaaaaaa

SEQ ID NO: 6 - DNA, 3228 bp, Bos taurus
acaggggaaaatgaagctgagcagaaggatctgttactcaggatcccaacccaagagtcccccttcctgaaccaaagtctcaagcacctttcaggggacaagaatccaagccattgcaaattagaagagaaacaaaagagcaaggagtaatgaggagaagggatgaggagaaggatgagaagtcaagagcaaagagaggctgacttaggtgactctggcgacagctggtgaagcagtgggtgatggcgtatctgctgcaaggccggctgcccatcaatcaagatctgctgctgatgcagaaaggcacaatgatgcgcaaggtgaggtccaaaagctggaagaagctaagattcttcagacttcaggacgatggcatgacagtctggcatgcccggcaggctggaggcagggccaagcccagcttctcaatctccgacgtggacacagtacgtgaggaccacgagtctgagttgctgcgcaacctggcagaggagttccccctggagcagggcttcaccatcgtcttccatggccgccgctccaacctggacctggtggccaacagtgtccaggaagcccagacctggatgcaggggctccagctgttggtgggctttgtcaccaacatggaccaacaggagaggctggatcaatggctgagtgattggttccagcgcggagacaaaaaccaggatggtcggatgagtttcggagaggtccaacggttattgcacctgatgaatgtggaaatggaccaggaatatgcctttcagcttttccagacagcagacacatcccagtctgggactctggagggagaagaatttgtagagttctataagtcattgactcagcgtccagaagtgcaggaactgtttgaaaagttttcatctgatgggcagaaactgaccctgttggaatttgtggatttcctccaagaggaacagaaggaaggagaacgggcttctgaccttgctctggaactcatcgaccgctatgagccttcagaaagtggaaaactgcggcatgtgctgagcatggatggcttcctcggctacctctgctcgaaggacggagacatcttcaatccaacctgccaccccctctaccaggacatgactcaacccctgaaccactactacatcaactcctcccacaacacctacctggtgggggaccagctttgtggccagagcagcgtggagggatacatacggaccctgaagcgggggtgccgctgtgtggaggtggatatatgggatggaccaagcggggaacctattgtttaccacggacacaccctgacctcccgcatcccattcaaagatgttgtggccgctataggacagtatgccttccagacgtcagactatccagtcatcttgtcgctggagaaccactgcagctgggagcagcaggagatcatcgtgcgccatctgacggagatcctgggggatcagctactgacgacagccttggatgggcagcctcccactcagctgccctcccccgaggaccttcgggggaagatccttgtgaaggggaagaagctaatgcttgaggaagaggaggaagaaccagaggctgaattggaagcagagcaggaggctaggctggacttggaggcccagctggagtctgagccccaggacctgagccctcgcagtgaggacaaaaagaaggttgtgatgtgcccgctgttgtgtcggcctttgtgttgtcagattatggcccaggctcctatttccaagcctgggcttcttctctttcccaagcagaaacccaaggccatcttgtgtccagccctctctgccctggttgtctacttgaaggctgtcacattctatagcttcactcactcgagggagcactaccacttctatgaaacatcatctttctctgaaaccaaggccaagagcctcatcaaggaggctggtgacgagtttgtgcagcacaatgcctggcagttaagcagagtgtatcccagtggcctgaggacggattcatccaactacaacccccaggaattctggaatgcaggctgccagatggtggctatgaacatgcagacagccgggctggagatggacctgtgtgatgggctcttccgccagaacgctggctgtggctatgtgctgaagccagacttcctgcgcgatgcccagagttccttccaccctgagaggcccatcagccctttcaaagcccagaccctcatcatccaggtaatcagtggtcagcaactccccaaagtggacaacaccaaagaacagtccattgtggaccccttggtgagagtggagatctttggcgtccgcccagacacaacccggcaagagaccagctacgtggagaacaatggttttaatccatactgggggcagacactgtgcttccggatcctggtacctgaactggccctgctgcgttttgtggtcaaagactatgactggaaatcccgaaacgacttcattggtcagtacaccctgccgtggagctgcatgcagcaaggttaccgccacatacacctgctctccaaggatggcctcagccttcacccagcttccatctttgtgcacatctgcacacaggaagtgtcagaggaggctgaatcttaaggcagccacttcacaggcggagtggattcactggctgtagatggtgagaaaaatgaaaaaaacctggaaggatgctccagaaagcaaacagtggtaaaaacccgagctttggactgtgccttctcaggcacaaaatgacttggtccttcctgacaagcaaatgtaggacccgatttttcaccttctttctcttcccattcttcgttttcacaagcttctggcatcattcctgtcctttccctctgcagactctacactggaattccttccttcctctggctataggctcaacaccatacccagatctaggacagtgctttcccaccaactctggctcaaaggcacattgcagctagaaatccagagaagggcttgggggcagagaaacccaaaacctaaacctccacccctaacttcctcaaagtccagagccaagggaaagacaaatcaagcctcaagtctccccaggcaaaggctggggaaggagatctgatcccctgtattatgactctcaagagaacactgcatggctctgagagtccccactggactcctgctcacttgtgccaccagtataaatagagcttctctctcaaaaaaaaaaaaaaaa